(19) United States
(12) Patent Application Publication
(10) Pub. No.: US 2023/0377714 A1
(43) Pub. Date: Nov. 23, 2023

(54) DEVICES, SYSTEMS, AND METHODS FOR OPTIMIZING MEDICAL PROCEDURES AND OUTCOMES

(71) Applicant: Orthosensor Inc., Dania Beach, FL (US)

(72) Inventors: Sally LIARNO, Bergenfield, NJ (US); Kevin ABBRUZZESE, Mahwah, NJ (US); Vincent ALIPIT, Tappan, NY (US); Andre FRELIGH, Glen Gardner, NJ (US)

(73) Assignee: Orthosensor Inc., Dania Beach, FL (US)

(21) Appl. No.: 18/320,832

(22) Filed: May 19, 2023

Related U.S. Application Data
(60) Provisional application No. 63/365,016, filed on May 19, 2022.

(51) Int. Cl. G16H 20/40; G16H 50/70; G16H 40/67; G16H 50/30; G16H 20/30; A61B 34/10; A61B 5/00

(52) U.S. Cl. CPC G16H 20/40; G16H 50/70; G16H 40/67; G16H 50/30; G16H 20/30; A61B 34/10; A61B 5/6801; A61B 2562/0219; A61B 2034/105; A61B 2034/104

(57) ABSTRACT

Aspects disclosed herein provide a method for optimizing a medical treatment plan. The method may include receiving kinematics data from a wearable sensor coupled to an instant patient, determining, based on the received kinematics data and stored information, a medical treatment plan. The procedure may include installation of an implant. Determining the medical treatment plan may include determining an alignment, position, design, or type of the implant. The stored information may include preoperative information for the instant patient and preoperative information, intraoperative information, and postoperative information from a plurality of previous patients having at least one characteristic in common with the instant patient. Each of the preoperative information, intraoperative information, and postoperative information may include kinematics data obtained using a previous wearable sensor.

### CROSS-REFERENCE TO RELATED APPLICATION

This application claims the benefit of priority under 35 U.S.C. § 119 to U.S. Provisional Patent Application No. 63/365,016, filed May 19, 2022, the entirety of which is incorporated herein by reference.

### FIELD OF THE DISCLOSURE

The present disclosure relates to systems and methods for optimizing medical procedures, and in particular to a system and a method for determining preoperative, intraoperative, and postoperative activities to optimize outcomes after joint replacement procedures.

### BACKGROUND OF THE DISCLOSURE

Musculoskeletal disease presents unique problems for medical practitioners. Surgeries incorporating prosthetics and/or implants such as joint replacement procedures often require careful consideration of various factors such as bone resection locations, soft-tissue tension, joint alignment, and joint balance for successful rehabilitation and use post-operation. Given the complex interrelationships of various patient-specific factors on surgery success, the more data a surgeon has about a patient, the more likely surgery can be successful. Improved computer and algorithmic systems and methods for performing, collecting, and analyzing data to assist in surgery are desired.

### BRIEF SUMMARY OF THE DISCLOSURE

In an aspect of the present disclosure, a method for optimizing a medical treatment plan may include receiving kinematics data from a wearable sensor coupled to an instant patient, determining, based on the received kinematics data and stored information, a medical treatment plan for the instant patient, and displaying the medical treatment plan on an electronic display. The procedure may include installation of an implant. Determining the medical treatment plan may include determining an alignment, position, design, or type of the implant. The stored information may include preoperative information for the instant patient, and preoperative information, intraoperative information, and postoperative information from a plurality of previous patients having at least one characteristic in common with the instant patient. Each of the preoperative information, intraoperative information, and postoperative information may include kinematics data obtained using a previous wearable sensor.

The kinematics data may include a range of motion, stiffness, or laxity of a first joint; and a range of motion, stiffness, or laxity of a second joint. The implant may be installed at the first joint.

The method may include determining that the stiffness of the second joint is greater than a predetermined stiffness for the second joint, or the laxity of the second joint is less than a predetermined laxity for the second joint. Determining the medical treatment plan may include determining whether the implant has a fit which is tighter than a predetermined fit based on the determined stiffness of the second joint and/or the determined laxity of the second joint.

Determining the medical treatment plan may include determining whether a slope of a bone to which the implant is to be aligned is to be less than a predetermined slope, a thickness of the implant is to be greater than a predetermined thickness, a number of tissue and/or bone cuts used during the procedure is to be less than a predetermined number, and/or the implant is to be a constrained type implant. The first joint may be a knee joint. The second joint may be a pelvic joint or hip joint. Determining the surgical plan may include determining that the implant is to be a valgus-valgus constrained (VVC) implant, and determining whether the implant is to be aligned with a tibial slope less than or equal to a predetermined tibial slope.

The kinematics data may include a range of motion, stiffness, or laxity of a first joint. The method may include receiving a bone density of a bone adjacent to the first joint.

The method may include determining whether the stiffness of the joint is below a predetermined stiffness threshold and/or that the laxity of the joint is above a predetermined laxity threshold, and determining whether the bone density is less than a predetermined bone density threshold. Determining the medical treatment plan may include determining whether the implant has a fit which is tighter than a predetermined fit.

The kinematics data may include postural sway or stability data of the instant patient, a number or frequency of bending motions, squat motions, lunge motions, sit-to-stand motions, or one-legged motions performed by the instant patient over a period greater than one day, and/or a number of fall events of the instant patient.

The method may include determining a fall risk score based on the kinematics data. Determining the medical treatment plan may be based on the fall risk score. The method may include determining whether the fall risk score is greater than a predetermined fall risk threshold. Determining the medical treatment plan may include determining that the implant is to be a constrained type of implant, and/or a number of tissue and/or bone cuts during the procedure is to be less than a predetermined number.

The method may include determining, based on the received kinematics data and stored information, a prehabilitation plan for the instant patient.

Receiving the kinematics data may include receiving additional kinematics data from a performance of the prehabilitation plan. Determining the prehabilitation plan may include determining, based on the additional kinematics data from the performance of the prehabilitation plan, a secondary prehabilitation plan. Determining the medical treatment plan may include determining, based on the additional kinematics data, a secondary medical treatment plan.

The method may include determining, based on the received kinematics data, a patient readiness score, and determining, based on the patient readiness score, a timing of performing the medical treatment plan, a timing of performing a prehabilitation plan, and/or a timing of performing a rehabilitation plan.

The method may include determining, based on the received kinematics data and stored information, a rehabilitation plan for the instant patient.

The method may include receiving additional data during a performance of the medical treatment plan. The implant may include one or more sensors. At least some of the additional data may be received from the implant.

In another aspect of the present disclosure, a method for optimizing a medical treatment plan may include receiving, after at least a portion of a medical treatment plan is performed, kinematics data from a sensored implant installed on an instant patient, and determining, based on the received kinematics data and stored information, a secondary medical treatment plan for the instant patient. Determining the secondary medical treatment plan may include determining an adjusted alignment, position, design, or type of the sensored implant. The stored information may include preoperative information for the instant patient, and preoperative information, intraoperative information, and postoperative information from a plurality of previous patients. Each of the preoperative information, intraoperative information, and postoperative information may include kinematics data obtained using a previous sensored implant.

The medical treatment plan may be for a total joint replacement surgery. The sensored implant may include an inertial measurement unit (IMU).

In yet another aspect of the present disclosure, a method for optimizing a medical treatment plan may include receiving primary data from a sensored implant installed on an instant patient during performance of the medical treatment plan, receiving from a robotic device, after at least a portion of the medical treatment plan is performed, secondary data including at least one of biometrics data, incision length data, soft tissue integrity data, pressure data, and/or implant position data, and determining, based on the primary data, the secondary data, and stored information, a secondary medical treatment plan for the instant patient. Determining the secondary medical treatment plan may include determining an alignment, position, design, or type of the sensored implant. The stored information may include preoperative information for the instant patient, and preoperative information, intraoperative information, and postoperative information from a plurality of previous patients. Each of the preoperative information, the intraoperative information, and the postoperative information may include primary data obtained using a previous sensored implant and secondary data obtained using a previous robotic device.

### BRIEF DESCRIPTION OF THE DRAWINGS

A more complete appreciation of the subject matter of this disclosure and the various advantages thereof may be realized by reference to the following detailed description, in which reference is made to the following accompanying drawings:

FIG. 1 is a flow chart illustrating a system for collection, transmission, and storage of preoperative, intraoperative, and postoperative data, and outputs of the system according to aspects of this disclosure.

FIG. 2 is a schematic diagram depicting the processing of preoperative, intraoperative, and postoperative data and outputs of the system of FIG. 1, according to aspects of this disclosure.

FIG. 3 is a schematic diagram exemplifying types of preoperative and intraoperative data and outputs of the system of FIG. 1, according to aspects of this disclosure.

FIG. 4 is a schematic diagram exemplifying types of postoperative data and outputs of the system of FIG. 1, according to aspects of this disclosure.

FIGS. 5 and 6 show exemplary legs and bones of a leg, respectively, and showing various mechanical axes which may be measured as part of kinematic data included in FIGS. 3-4, according to aspects of this disclosure.

FIG. 7 illustrates a preoperative measurement system configured to collect preoperative data, according to aspects of this disclosure.

FIGS. 8-9 illustrate exemplary wearable sensors, according to aspects of this disclosure.

FIG. 10 illustrates an intraoperative measurement system configured to collect intraoperative data, according to aspects of this disclosure.

FIGS. 11-13 illustrate exemplary sensored medical devices, according to aspects of this disclosure.

FIGS. 14-18 illustrate exemplary displays of a graphical user interface, according to aspects of this disclosure.

FIG. 19 illustrates a postoperative measurement system configured to collect postoperative data, according to aspects of this disclosure.

FIGS. 20-21 illustrate an exemplary sensored patient bed, according to aspects of this disclosure.

FIG. 22 is a flow chart illustrating exemplary preoperative, intraoperative, and postoperative algorithms for the system of FIG. 1, according to aspects of this disclosure.

FIG. 23 is a flow chart illustrating an exemplary method, according to aspects of this disclosure.

FIG. 24 is a flow chart illustrating an exemplary method for determination of a prehabilitation exercise program, according to aspects of this disclosure.

FIG. 25 is a flow chart illustrating an exemplary method for determination of a patient readiness score, according to aspects of this disclosure.

FIG. 26 is a flow chart illustrating an exemplary method or determination of an alignment of a medical device based on fall risk, according to aspects of this disclosure.

FIG. 27 is a flow chart illustrating an exemplary method for determination of an alignment of a medical device (e.g., implant) based on bone density and kinematics data, according to aspects of this disclosure.

FIG. 28 is a flow chart illustrating an exemplary method for determination of an alignment of an implant based on kinematics data of multiple joints, according to aspects of this disclosure.

FIG. 29 is a flow chart illustrating an exemplary method for determination of an alignment of an implant based on a finite element analysis, according to aspects of this disclosure.

FIG. 30 is a flow chart illustrating an exemplary method for determination of a postoperative exercise plan, according to aspects of this disclosure.

FIG. 31 is a flow chart illustrating an exemplary method for optimizing a postoperative exercise plan, according to aspects of this disclosure.

FIG. 32 is a flow chart illustrating an exemplary method for determination of a pain medication plan, according to aspects of this disclosure.

FIG. 33 is a flow chart illustrating an exemplary method for determination of a discharge plan, according to aspects of this disclosure.

### DETAILED DESCRIPTION

Reference will now be made in detail to the various embodiments of the present disclosure illustrated in the accompanying drawings. Wherever possible, the same or like reference numbers will be used throughout the drawings to refer to the same or like features. It should be noted that the drawings are in simplified form and are not drawn to precise scale. Additionally, the term “a,” as used in the specification, means “at least one.” The terminology includes the words above specifically mentioned, derivatives thereof, and words of similar import. Although at least two variations are described herein, other variations may include aspects described herein combined in any suitable manner having combinations of all or some of the aspects described.

As used herein, the terms “implant trial” and “trial” will be used interchangeably and as such, unless otherwise stated, the explicit use of either term is inclusive of the other term. In this disclosure, “user” is synonymous with “practitioner” and may be any person completing the described action (e.g., surgeon, technician, nurse, etc.).

An implant may be a device that is at least partially implanted in a patient and/or provided inside of a patient's body. For example, an implant may be a sensor, artificial bone, or other medical device coupled to, implanted in, or at least partially implanted in a bone, skin, tissue, organs, etc. A prosthesis or prosthetic may be a device configured to assist or replace a limb, bone, skin, tissue, etc. Many prostheses are implants, such as a tibial prosthetic component. Some prostheses may be exposed to an exterior of the body and/or may be partially implanted, such as an artificial forearm or leg. Some prostheses may not be considered implants and/or otherwise may be fully exterior to the body, such as a knee brace. Systems and methods disclosed herein may be used in connection with implants, prostheses that are implants, and also prostheses that may not be considered to be “implants” in a strict sense. Therefore, the terms “implant” and “prosthesis” will be used interchangeably and as such, unless otherwise stated, the explicit use of either term is inclusive of the other term. Although the term “implant” is used throughout the disclosure, this term should be inclusive of prostheses which may not necessarily be “implants” in a strict sense.

In describing preferred embodiments of the disclosure, reference will be made to directional nomenclature used in describing the human body. It is noted that this nomenclature is used only for convenience and that it is not intended to be limiting with respect to the scope of the invention. For example, as used herein, the term “distal” means toward the human body and/or away from the operator, and the term “proximal” means away from the human body and/or towards the operator. As used herein, the terms “comprises,” “comprising,” or any other variation thereof, are intended to cover a non-exclusive inclusion, such that a process, method, article, or apparatus that comprises a list of elements does not include only those elements, but may include other elements not expressly listed or inherent to such system, process, method, article, or apparatus. The term “exemplary” is used in the sense of “example,” rather than “ideal.” Further, relative terms such as, for example, “about,” “substantially,” “approximately,” etc., are used to indicate a possible variation of ±10% in a stated numeric value or range.

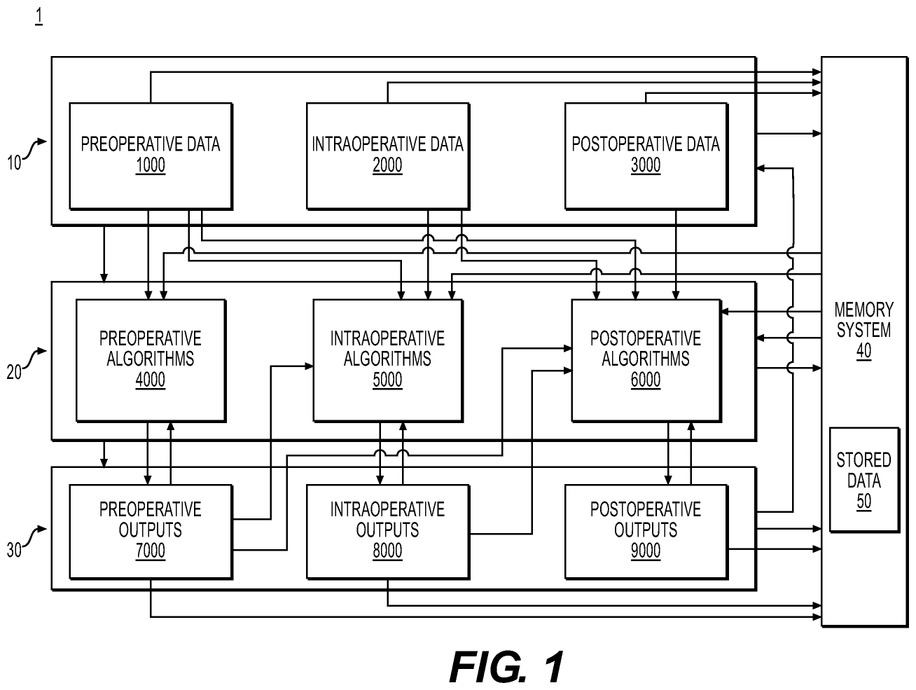

FIG. 1 illustrates an electronic data processing system 1 for collecting, storing, processing, and outputting data throughout the course of treatment of a patient.

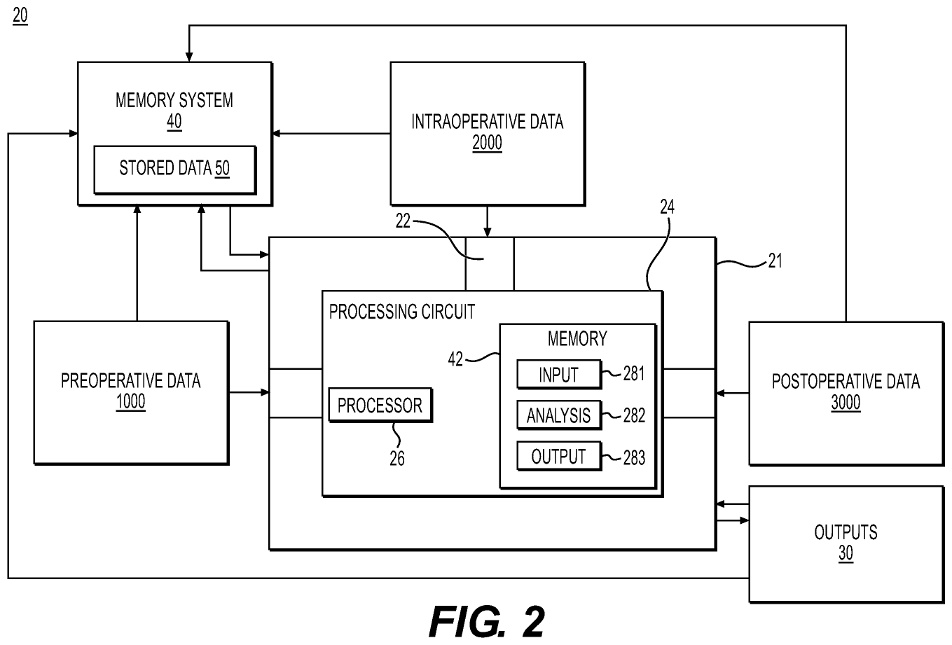

Referring to FIG. 1, input information 10 may be input into a system or module 20 to generate output information 30, which may be fed back into system 20 as input information 10. System 20 may be an artificial intelligence (AI) and/or machine learning system. System 20 may include an AI module 21 (shown in FIG. 2), which may include or communicate with a memory system 40 configured to store the plurality of inputs or input information 10, outputs or output information 30, and stored data 50 from prior patients and/or prior procedures. The input information 10 and output information 30 of an instant procedure may also become stored data 50 and/or used as input information 10 into system 20 and/or memory system 40. Although certain information is described in this specification as being input information 10 or output information 30, due to the continuous feedback loops of data (which may be anchored by memory system 40), the input information 10 described herein may alternatively be determinations or output information 30, and the output information 30 described herein may also be used as input information 10. For example, some input information 10 may be directly sensed or otherwise received, and other input information 10 may be determined or output based on other input information 10.

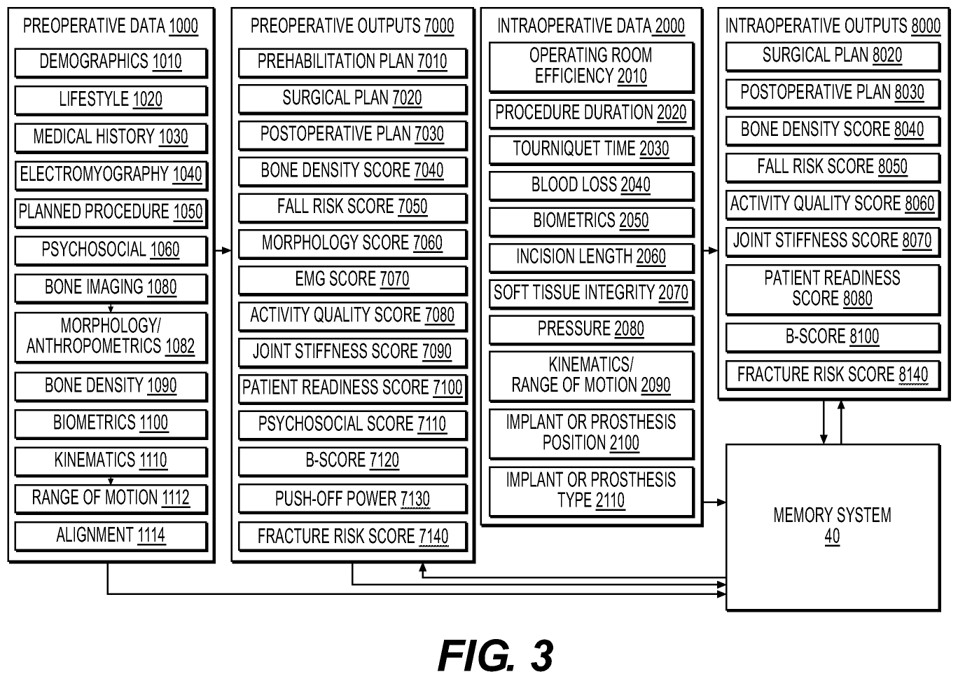

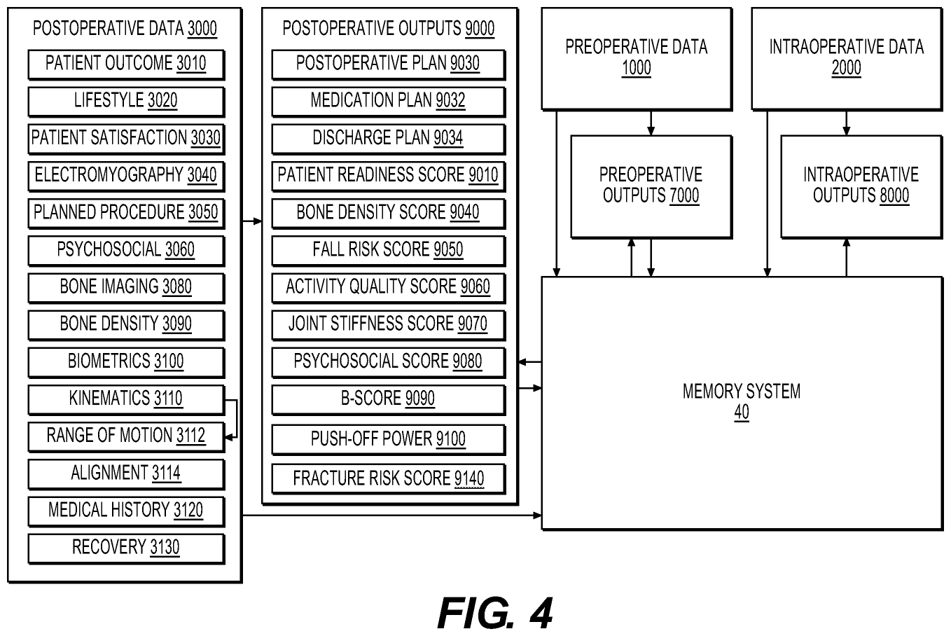

The input information 10 may include preoperative data 1000, intraoperative data 2000, and post-operative data 3000. System 20 may perform a plurality of algorithms, such as preoperative algorithms 4000, intraoperative algorithms 5000, and postoperative algorithms 6000 to generate the output information 30. The output information 30 may include preoperative outputs 7000, intraoperative outputs 8000, and postoperative outputs 9000. Some or all of the preoperative outputs 7000, intraoperative outputs 8000, and postoperative outputs 9000 may include determinations such as guidance for medical procedures, guidance for pre-operative or pre-habilitation treatment plans, guidance for post-operative or recovery plans, etc., as will be described in more detail hereinafter. System 20 may include one or more algorithms or modules configured to aggregate results from multiple preoperative algorithms 4000, intraoperative algorithms 5000, and/or postoperative algorithms 6000 to compile algorithm determinations for certain outputs (e.g., surgical plans, medical treatment plans, or instructions). As shown by the arrows in FIG. 1, the preoperative outputs 7000, intraoperative outputs 8000, and postoperative outputs 9000 may become inputs into system 20 and/or memory system 40. Details of the input information 10 and output information 30 will be described with reference to FIGS. 4-3.

Preoperative data 1000 may be data collected, received, and/or stored prior to an initiation of a medical treatment plan or medical procedure. Intraoperative data 2000 may be data collected, received, and/or stored during a medical treatment plan or medical procedure. Although the term “intraoperative” is used, the word “operative” should not be interpreted as requiring a surgical operation. Postoperative data 3000 may data be collected, received, and/or stored after completion of the medical treatment or medical procedure.

FIG. 2 illustrates an exemplary system architecture for system 20. Referring to FIG. 2, the AI module 21 may be implemented using one or more computing platforms. Examples of one or more computing platforms may include, but are not limited to, smartphones, wearable devices, tablets, laptop computers, desktop computers, Internet of Things (IoT) device, remote server/cloud based computing devices, or other mobile or stationary devices. The AI module 21 may also include one or more hosts or servers connected to a networked environment through wireless or wired connections. Remote platforms may be implemented in or function as base stations (which may also be referred to as Node Bs or evolved Node Bs (eNBs)). Remote platforms may also include web servers, mail servers, application servers, etc.

The AI module 21 may include at least one communication module or interface 22 and a processing circuit 24. The processing circuit 24 may include one or more processors 26 and a memory or storage 42. The memory or storage 42 may be a part of the memory system 40. The memory system 40 is shown in FIG. 2 as providing separate storage from the AI module 21 to exemplify that large amounts of data (e.g., stored data 50) may be stored separately and sent to the AI module 21 via communication modules 22 when needed or where appropriate. However, the memory system 40 may be a part of a computing platform for the AI module 21.

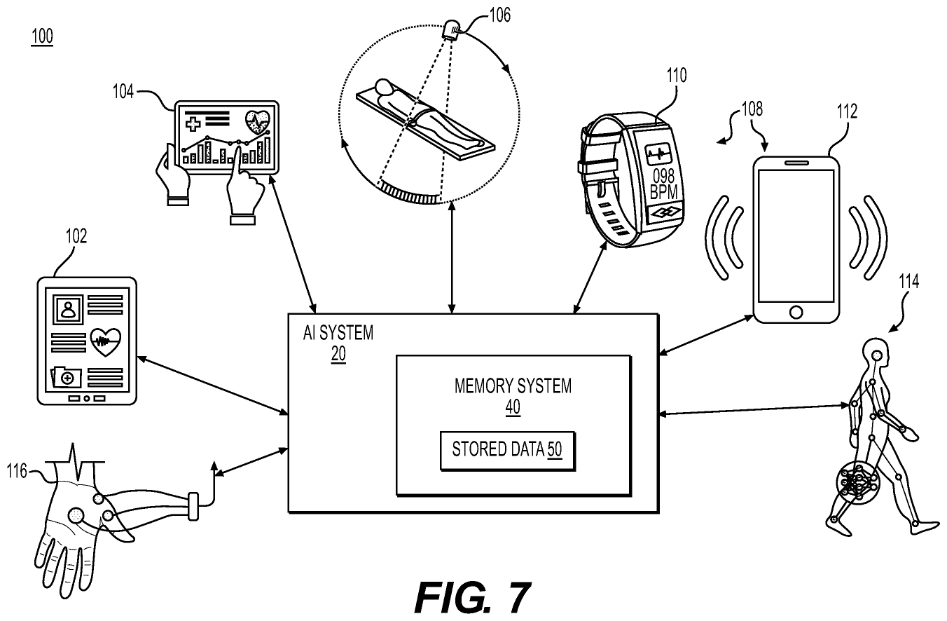

The AI module 21 may be configured to receive the plurality of inputs 10 (the preoperative data 1000, intra-operative data 2000, and post-operative data 3000), and/or stored data 50 from prior procedures or patients, via the communication module 22. The preoperative data 1000, intra-operative data 2000, and post-operative data 3000 may be received via manual input or from the various sensors discussed with references to FIGS. 7-14. The plurality of inputs 10 may be stored in memory 42 and/or memory system 40. The plurality of input information 10 may be analyzed by processor 24 to determine patterns. The AI module 21 may be configured to perform the preoperative algorithms 4000, intraoperative algorithms 5000, and postoperative algorithms 6000 via the processing circuit 24, and to generate the output information 30 via the processor 26.

The communication module 22 may enable wireless communications between the system 20 and the various sensors or data collection devices described herein. The communication module 22 may include wired or wireless interfaces (e.g., jacks, antennas, transmitters, receivers, transceivers, wire terminals, etc.) for conducting data communications with external sources via a direct connection or a network connection (e.g., an Internet connection, a LAN, WAN, or WLAN connection, LTE, 4G, 5G, Bluetooth, near field communication (NFC), radio frequency identifier (RFID), ultrawideband (UWB), etc.). The communication module 22 may include a radio interface including filters, converters (for example, digital-to-analog converters and the like), mappers, a Fast Fourier Transform (FFT) module, and the like, to generate symbols for a transmission via one or more downlinks and to receive symbols (for example, via an uplink). The communication module 22 may include a BlueTooth module, WiFi module, etc. to receive the input information 10. For example, communication module 22 may include an Ethernet card and port for sending and receiving data via an Ethernet-based communications link or network. In another example, communication module 22 may include a Wi-Fi transceiver for communication via a wireless communications network.

The processing circuit 24 may be configured to implement various functions (e.g., calculations, processes, analyses) described herein. The processor 26 may be implemented as a general purpose processor or computer, special purpose computer or processor, microprocessor, digital signal processor (DSPs), an application specific integrated circuit (ASIC), one or more field programmable gate arrays (FPGAs), a group of processing components, processor based on a multi-core processor architecture, or other suitable electronic processing components. The processor 26 may be configured to perform machine readable instructions, which may include one or more modules implemented as one or more functional logic, hardware logic, electronic circuitry, software modules, etc. In some cases, the processor 26 may be remote from one or more of the computing platforms comprising the module 21 and/or system 20. The processor 26 may be configured to perform one or more functions associated with the AI module 21, such as precoding of antenna gain/phase parameters, encoding and decoding of individual bits forming a communication message, formatting of information, and overall control of one or more computing platforms comprising the AI module 21, including processes related to management of communication resources and/or the communication module 22.

The memory 42 may provide an example of the types of devices comprising the memory system 40. The memory 42 may be one or more external or internal devices (random access memory or RAM, read only memory or ROM, Flash-memory, hard disk storage or HDD, solid state devices or SSD, static storage such as a magnetic or optical disk, other types of non-transitory machine or computer readable media, etc.) configured to store data and/or computer readable code and/or instructions that completes, executes, or facilitates various processes or instructions described herein. The memory 42 may be or include volatile memory or non-volatile memory (e.g., semiconductor-based memory device, a magnetic memory device and system, an optical memory device and system, fixed memory, or removable memory). The memory 42 may include database components, object code components, script components, or any other type of information structure to support the various activities described herein. In some aspects or embodiments, the memory 42 may be communicably connected to the processor 26 and may include computer code to execute one or more processes described herein. The memory 42 may contain a variety of modules, each capable of storing data and/or computer code related to specific types of functions. In some embodiments, the memory 42 may contain several modules related to medical procedures, such as an input module 281, an analysis module 282, and an output module 283. The input module 281 may receive input information 10, and the output module 283 may output (e.g., display or transmit) output information 30. The analysis module 282 may include and/or operate the preoperative algorithms 4000, intraoperative algorithms 5000, and postoperative algorithms 6000.

The AI module 21 and/or system 20 need not be contained in a single housing. Rather, components of the AI module 21 may be located in various different locations or even in a remote location. Components of the module 21, including components of the processor 26 and the memory 42, may be located, for example, in components of different computers, robotic systems, devices, etc. used in surgical procedures.

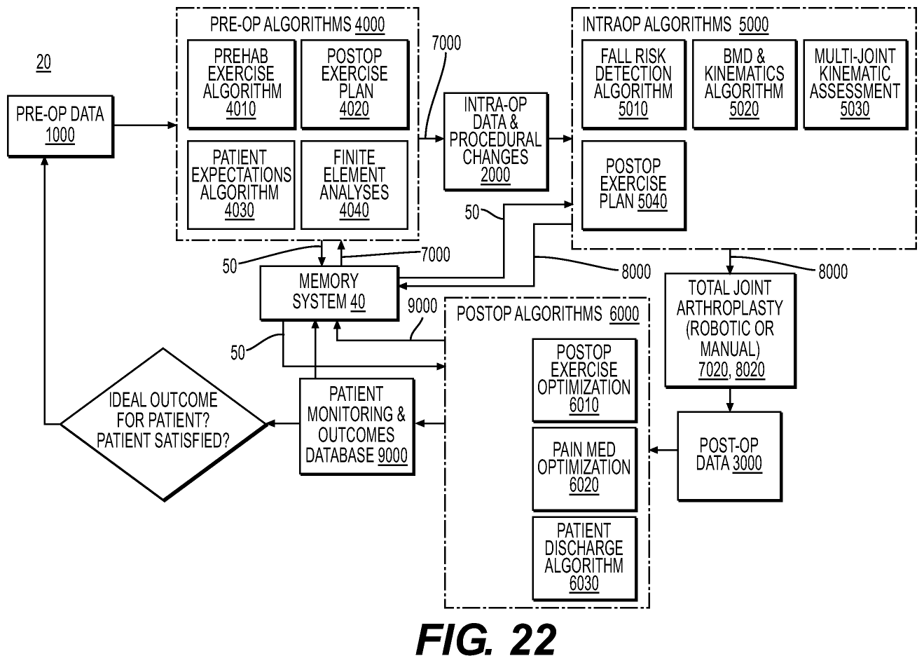

FIGS. 3-4 illustrate the types of input information 10 and output information 30, and FIGS. 7-14 illustrate examples of various output information 30 and how various other input information 10 may be measured. The pre-operative data 1000, intra-operative data 2000, and post-operative data 3000 may be collected using preoperative, intraoperative, and postoperative measurement systems 100, 200, and 300. The preoperative algorithms 4000, intraoperative algorithms 5000, and postoperative algorithms 6000 may be used to generate preoperative outputs 7000, intraoperative outputs 8000, and postoperative outputs 9000. Exemplary processes and algorithms utilizing systems 100, 200, and 300 are described in detail beginning with FIG. 22.

Preoperative Data 1000

Preoperative data 1000 may include any information collected by memory system 40 prior to a medical procedure, such as a surgical procedure or other patient treatment event. Referring to FIGS. 3-4, the preoperative data 1000 may include information on demographics 1010, lifestyle 1020, medical history 1030, electromyography (EMG) 1040, planned procedure 1050, psychosocial information 1060, bone imaging 1080, bone density 1090, biometrics 1100, and kinematics 1110. This list, however, is not exhaustive and preoperative data 1000 may include other patient specific information. Some of the preoperative data 1000 may be directly sensed via one or more devices, may be manually entered by a medical professional, patient, or other party, and other preoperative data 1000 may be determined (e.g., using a preoperative algorithm 4000) based on directly sensed information, input information, and/or stored information from prior medical procedures.

Demographics 1010 may include patient age, gender, height, weight, nationality, body mass index (BMI), etc. Lifestyle 1020 may include information on smoking habits, exercise habits, drinking habits, eating habits, fitness, thrill-seeking habits and/or risk adverse traits, a type of vehicle a patient drives and movements associated with entering and exiting the vehicle, a type of house or residence the patient lives in and movements associated with climbing and descending stairs, bending movements during daily activities, etc.

Medical history 1030 may include allergies, disease progressions, addictions, prior medication use, prior drug use, prior infections, comorbidities, prior surgeries or treatment, prior injuries, prior pregnancies, utilization of orthotics, braces, prosthetics, or other medical devices, etc. EMG information 1040 may include information on a muscle response or electrical activity in response to a nerve's stimulation.

Information on a planned procedure 1050 may include information about a planned site of the procedure, a disease or infection state, type of procedure to be performed, etc. Alternatively or in addition thereto, a planned procedure 1050 may include a surgeon's surgical or other procedure or treatment plan (planned steps or instructions on incisions, bone cuts, implant design, implant alignment, etc.) that was manually prepared by a surgeon and/or previously prepared using one or more algorithms. Psychosocial information 1060 may include perceived pain, stress level, anxiety level, mental health status, other feelings and psychosocial data, and other patient reported outcome measures (PROMS). Pyschosocial information 1060 may include mental health status and/or information from a Veteran's Rand-12 (VR-12) mental component summary (MCS).

Bone imaging data 1080 may include morphology and/or anthropometrics 1082 (e.g., physical dimensions of internal organs, bones, etc.), fractures, slope or angular data, tibial slope, posterior tibial slope or PTS, bone density 1090 (e.g., bone mineral or bone marrow density, bone softness or hardness, or bone impact), etc. Bone density 1090 may be collected separately from bone imaging information 1080 and/or may be collected using, for example, using indent tests or a microindentation tool. Bone imaging data 1080 may not be limited to strictly “bone” and may be inclusive of other internal imaging data, such as of cartilage, soft tissue, or ligaments.

Bone imaging data 1080 may include or be used to determine alignment data 1114. Bone imaging data 1080, alignment data 1114, and/or morphology and/or anthropometrics 1082 may include data on bone landmarks (e.g., condyle surface, head or epiphysis, neck or metaphysis, body or diaphysis, articular surface, epiconcyle, process, protuberance, tubercle vs tuberosity, trochanter, spine, linea or line, facet, crests and ridges, foramen and fissure, meatus, fossa and fovea, incisure and sulcus, and sinus) and/or bone geometry (e.g., diameters, slopes, angles) and other anatomical geometry data. Such geometry is not limited to overall geometry and may include specific lengths or thicknesses (e.g, lengths or thicknesses of a tibia or femur). Bone imaging data 1080, alignment data 1114, and/or morphology and/or anthropometrics 1082 may also include data on soft tissues for ligament insertions and/or be used to determine ligament insertion sites. For example, bone density 1090 may be determined from bone imaging data 1080 and may be used to locate or determine a ligament insertion site to balance a knee.

Bone imaging data 1080, alignment data 1114, and/or morphology and/or anthropometrics 1082 may include lower extremity mechanical alignment, lower extremity anatomical alignment, femoral articular surface angle, tibial articular surface angle, mechanical axis alignment strategy, anatomical alignment strategy, natural knee alignment strategy, femoral bowing, tibial bowing, patello-femoral alignment, coronal plane deformity, coronal plane deformity that can be passively correctable, sagittal plane deformity, extension motion, flexion motion, anterior cruciate ligament (ACL) ligament intact, posterior cruciate ligament (PCL) ligament intact, knee motion in all three planes during active and passive range of motion in a joint, three dimensional size, proportions and relationships of joint anatomy in both static and motion, height of a joint line, lateral epicondyle, medial epicondyle, lateral femoral metaphyseal flare, medial femoral metaphyseal flare, proximal tibio-fibular joint, tibial tubercle, coronal tibial diameter, femoral interepicondylar diameter, femoral intermetaphyseal diameter, sagittal tibial diameter, posterior femoral condylar offset-medial and lateral, lateral epicondyle to joint line distance, and/or tibial tubercle to joint line distance.

Biometrics 1100 may include resting heart rate or heat rate variability, electrocardiogram data, breathing rate, temperature (e.g., internal or skin temperature), skin moisture, oxygenation, sleep patterns (e.g., heart rate variability or HRV, REM cycle data, type of sleep such as REM, deep, or light, sleep frequency, time asleep versus time awake, disturbances in the sleep or periods of movement, patterns in sleep timing or time of day asleep, etc.), and/or activity frequency and intensity. Biometrics 1100 may include patient-specific or unique characteristics, such as fingerprint data, DNA or RNA signatures, etc.

Kinematics 1110 may include movement or position information at various anatomical areas or locations, muscle function or capability, and range of motion 1112 data. Additional kinematics 1110 data may include strength measurements and/or force measurements. For example, kinematics 1110 may include data used to determine a push-off power, force, or acceleration, or a power, force, or acceleration at a toe during walking. Range of motion 1112 data may include a range of motion at one or more joints, such as an angular range or axes of joint motion, or flexion or extension data. For example, kinematics 1110 may include a flexion value, where a flexion value of 180 degrees±3 degrees may indicate a full extension of a joint, and any value other than 180 degrees±3 degrees may indicate a joint in flexion where bones on either side of the joint intersect to form an angle other than 180 degrees. Kinematics 1110 may include dynamic information, speed or acceleration information, torque or force information, etc. Some of this information may be estimated or determined based on raw data from motion sensor systems 114 and/or other sensors. For example, kinematics 1110 may include how quickly a patient can bend a joint, sit down, stand up, a push-off power during walking, etc. Kinematics 1110 may also include steps (e.g., measured by a pedometer) and/or measured gait. Kinematics 1110 may include a number of fall events and/or disoriented events (e.g., measured by an accelerometer, mobile device 108, etc.)

Kinematics 1110 may include swaying or other movement which would indicate an unsteady balance of a patient, such as postural sway at the hips, knees, or neck. Kinematics 1110 may include pendulum knee drop information. Kinematics 1110 may also include and/or indicate frailty, fall risk, and/or joint stiffness (e.g., based on a speed or ease of how a joint is moved through a range of motion).

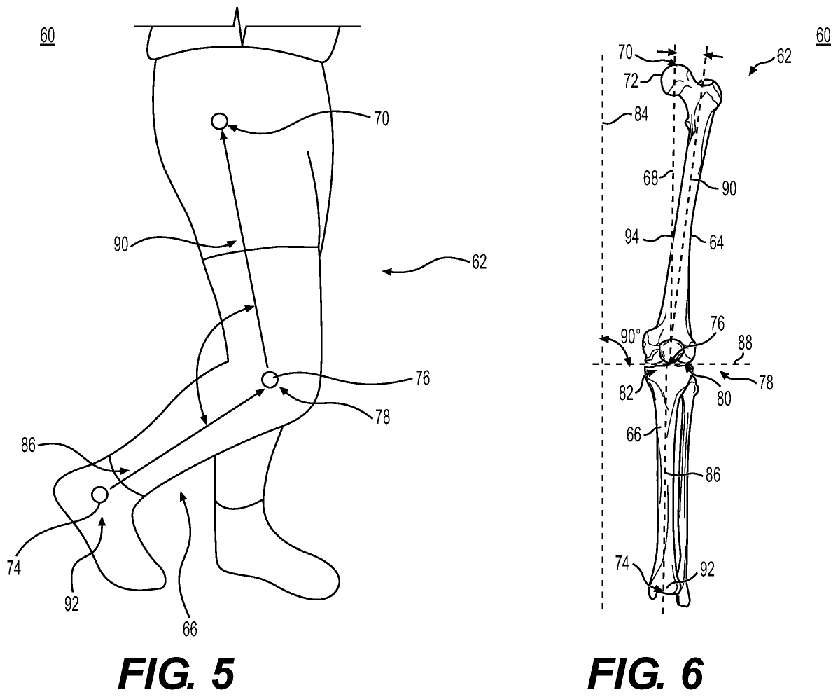

The kinematics information 1110 may include measurements in relation to a leg axis system 60 (FIGS. 5-6), such as alignment data 1114 or other anatomical measures. Alignment data 1114 may be obtained using kinematics information 1110 and/or range of motion information 1112, bone imaging data 1080 and/or morphology/anthropometrics data 1082, etc. In this way, alignment data 1114 may also be a type of preoperative output 7000. Anatomical measures and/or alignment data 1114 may include arithmetic hip-knee-ankle angle or aHKA, anatomical hip-knee-ankle angle, medial proximal tibial angle or MPTA, lateral distal femoral angle or LDFA, mechanical axis alignment, anatomic alignment, natural knee alignment, gap balancing, measured resection, etc., and these values may be combined. For instance, a joint line may be a sum of MPTA and LDFA, and a hip-knee-ankle angle or HKA may be a difference between MPTA and LDFA. These values may be used as coordinates on a 2D plane to describe a patient's knee anatomy.

Preoperative data 1000 and/or information stored in the memory system 40 may also include known data and/or data from third parties, such as data from the Knee Society Clinical Rating System (KSS) or data from the Western Ontario and McMaster Universities Osteoarthritis Index (WOMAC).

FIGS. 5-6 illustrate the leg axis system 60. However, aspects disclosed herein are not limited to enhancing alignment of a leg or a knee joint, and may enhance alignment and/or functions at other joints or body parts. Referring to FIGS. 5-6, the leg axis system 60 may be relative to a leg 62. Leg 62 may be a right or left leg. Leg 62 may comprise a femur 64 and a tibia 66. A mechanical axis 68 of leg 62 may be illustrated by a dashed line drawn through a center 70 of a femoral head 72 (at a hip joint) to a center 74 of ankle joint 96. The mechanical axis 68 may extend through a center 76 of a knee joint 78 at approximately a medial tibial spine 94. The knee joint 78 may comprise lateral articular surfaces and/or compartments 80 and medial articular surfaces and/or compartments 82 that support leg movement. Alignment of leg 62 to the mechanical axis 68 may minimize wear on articular surfaces of a prosthetic knee joint and may reduce mechanical stress on the wear surfaces of the femur 64 and tibia 66. Similarly, alignment to the mechanical axis 68 of leg 62 may reduce stress on any prosthetic components coupled to the femur 64 and/or tibia 66. Alignment of the knee joint 78 may further include balancing between lateral compartment 80 and medial compartment 82 of the knee joint 78.

A vertical axis 84 is shown by a dashed line drawn relative to the mechanical axis 68 and an anatomical axis 86 of the tibia 66. A horizontal axis 88 is shown by a dashed line that is perpendicular to the vertical axis 84. The horizontal axis 88 is shown extending through center 76 of knee joint 78 between a distal end of femur 64 and a proximal end of tibia 66. The vertical axis 84 may align with the pubic symphysis, which is a midline cartilaginous joint in proximity to a pelvic region. An anatomical axis 90 of the femur 64 is illustrated by dashed line 90. The anatomical axis 90 of the femur 64 may traverse an intramedullary canal of femur 64. The anatomical axis 86 of the tibia 66 may traverse an intramedullary canal of tibia 66. The mechanical axis 68 and the anatomical axis 86 of the tibia 66 may lie along a same line or be the same from the knee joint 78 to the center 74 of the ankle joint 76 of the leg 62.

The femur 64 and tibia 66 can be misaligned to the mechanical axis 68 of the leg 62. In an aligned leg, the mechanical axis 68 may form an angle of approximately 3 degrees with the vertical axis when the leg is fully extended. A surgeon may install prosthetic components in a knee joint 78 aligned to the mechanical axis 68 of the leg 62 to optimize reliability and performance of the knee joint 78. An alignment process may include measurement of leg misalignment (e.g. the offset of the anatomical axis 90 from the mechanical axis 68) and determination of the required compensation to align leg 62 to the mechanical axis 68 within a predetermined range. The predetermined range may be determined by a prosthetic component manufacturer or a medical practitioner based on clinical evidence that supports reliability and performance of the knee joint 78 when misalignment is kept within the predetermined range.

### Preoperative Measurement System 100

Referring to FIGS. 2 and 7, the system 20 may collect pre-operative data 1000 from the preoperative measurement or sensing system 100. The preoperative measurement system 100 may include electronic devices storing electronic medical records (EMR) 102, patient/user interfaces or applications 104 such as tablets, computers, and cellular phones 112, diagnostic imaging systems 106, mobile devices 108, a motion sensor, pressure sensor, and/or kinesthetic sensing systems 114 (see paragraph [0065] et seq.), and electromyography or EMG systems 116. The devices of the preoperative measurement system 100 may each include one or more communication modules (e.g., WiFi modules, BlueTooth modules, etc.) configured to transmit preoperative data 1000 to the memory system 40, the system 20, to each other, etc. The system 20 may use other types of stimulation systems (e.g., configured for a kinematic or EMG response) to collect preoperative data 1000.

The system 20 may collect patient reported data, practitioner assessments, etc. using EMR 102. For example, EMR 102 may be used to collect data on demographics 1010, medical history 1020, biometrics 1100, and information about a planned procedure 1050. Patient and/or user interfaces 104 may be implemented on mobile applications and/or patient management websites or interfaces such as OrthologIQ®. Patient interfaces 104 may present questionnaires, surveys, or other prompts for patients to enter psychosocial information 1060 such as perceived or evaluated pain, stress level, anxiety level, feelings, and other patient reported outcome measures (PROMS). Practitioners may also report psychosocial information 1060 (e.g., qualitative assessments or evaluations) via patient interfaces 104 or other interfaces. Patients may also report lifestyle information 1020 via patient interfaces 104. These patient interfaces 104 may be executed on other devices disclosed herein (e.g., using mobile devices 108 or other computers).

The system 20 may collect imaging information from diagnostic imaging systems 106, which may include computed tomography (CT) scans, magnetic resonance imaging (MM), x-rays, radiography, ultrasound, thermography, tactile imaging, elastography, nuclear medicine functional imaging, positron emission tomography (PET), single-photon emission computer tomography (SPECT), etc. The system 20 may use these diagnostic imaging systems 106 to collect bone imaging information 1080, including morphology and/or anthropometrics 1082 fractures, and bone density 1090 (e.g., bone mineral density or bone marrow density).

Mobile devices 108 may include smartwatches 110, smartphones 112, tablets, and other devices known in the art. Mobile devices 108 may execute patient interfaces 104. In some examples, mobile devices 108 may include sensors and/or applications, which the system 20 may use to collect biometrics 1100 and other types of patient specific data. For example, mobile devices 108 (e.g., FitBit, Apple Watch, Hexoskin, Polaris strap, iPhone, etc.) may use cameras, light sensors, barometers, global positioning systems (GPS), accelerometers, temperature sensors (e.g., battery temperature sensors), and/or pressure sensors. In some examples, mobile devices 108 may measure heart rate, electrocardiogram data, breathing rate, temperature, oxygenation, sleep patterns, and also activity frequency and intensity.

The system 20 may use EMG systems 116 to collect EMG data 1040. EMG systems 116 may include one or more electrode attached to skin or muscle to measure electrical activity and/or responses to nerve stimulation. The system 20 may use EMG data 1040 to determine nerve damage or disease information. EMG data 1040 may include information on muscle activity of various body segments including knee, hip, ankle, tibialis anterior, foot, lower back, shoulder, wrist, elbow, forearm, neck, etc.

The system 20 may use motion sensor and/or kinesthetic sensing systems 114, which may include motion capture (mocap) systems, external motion sensors, wearable sensors, and/or sensors machine vision (MV) technology. Motion sensor systems 114 may measure motion using an optical or light sensor, an accelerometer, a gyroscope, a magnetometer, a compass, barometer, a global positioning system (GPS), a pressure sensor, etc.

The system 20 may use motion capture systems, which may include markerless motion capture systems and other motion sensors (e.g., wearable sensors) to collect kinematics and range of motion data. External motion sensors may include cameras, optical sensors, infrared sensors, ultrasonic sensors, etc. mounted, for example, in an operating room to monitor motion, heat, etc.

Wearable sensors 114 may include heart-rate monitors, some mobile devices 108 (e.g., smartwatch 110), and other sensor systems configured to be worn by a patient and track movement (e.g., travel movement and kinematics of anatomy, such as joint motion). Wearable sensors 114 may include accelerometers, GPS chips, acoustical ranging, magnetometer, inclinometers, hybrid sensors, MEMS devices, etc. Wearable sensors 114 may also include MotionSense sensors, ZipLine sensors, and/or pedometers. Wearable sensors 114 may monitor more than motion, such as pressure, temperature, sweat/perspiration, input related to stress, input related to air circulation, air purity or quality of an environment, etc. Wearable sensors 114 may include pressure insole sensors and/or sensored shoes configured to measure pressure, a pressure distribution, a center of pressure, etc. when a user steps. Such wearable sensors 114 may also measure acceleration or force as a user lifts a leg to take a step. Pressure data from pressure insole sensors or sensored shoes may be used to determine or evaluate balance, heel strike, and/or push-off forces, which may be used to determine or evaluate frailty, fall risk, compensatory gait, and overall function.

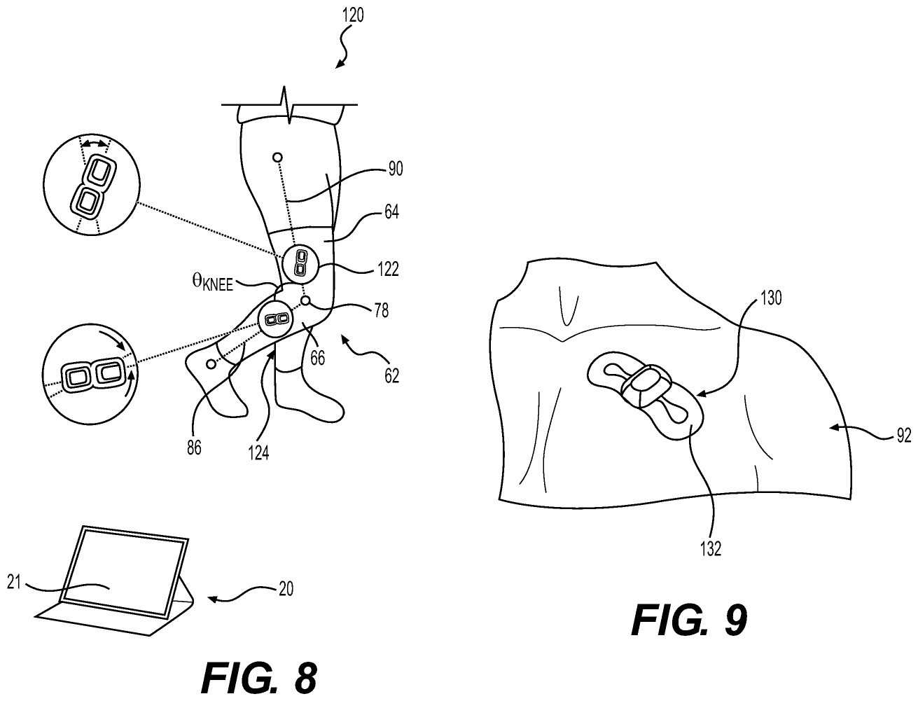

Referring to FIGS. 8-9, wearable sensors 114 may be implemented as a kinematics tracking system 120 and/or 130. As shown in the example of FIG. 8, the kinematics tracking system 120 and/or 130 may be implemented as a tracking system 120 for a leg 62 (see also FIGS. 5-4). The tracking system 120 may include a first device 122 and a second device 124. The system 20 and/or the tracking system 120 may include a computer 21 having a display 28.

The first device 122 may be coupled (e.g., adhered) to a first portion of a musculoskeletal system. The second device 124 may be coupled to a second portion of the musculoskeletal system. As shown in FIG. 8, the first device 122 may be coupled to a thigh or femur 64 to move with the femur 64. The second device 124 may be coupled to a calf or tibia 66 to move with the tibia 66.

The first and second devices 122 and 124 may be configured to measure a relative orientation between the first and second portions to determine an angle, such as an angle of knee joint 78 between the thigh and half. Since an orientation of first device 122 and/or second device 124 relative to a common fixed reference frame (earth, gravity) may be known, an angle of a joint (e.g., knee joint 78) coupling the first and second musculoskeletal portions (exemplified in FIG. 8 by a knee angle θknee) may be determined. Each of the first and second devices 122 and 124 may be calibrated based on an offset angle or calibration pose (e.g., when a person stands in a neutral anatomical position) to assist in measurement. For a knee joint 78, this calibration pose may occur at full extension of leg 62. Calibration may also be based on any known misalignments of the femur 64 with respect to the tibia 66.

The first and second devices 122 and 124 may include electronic circuitry and at least one sensor to measure orientation, pitch and/or roll of the sensor or a relative orientation, pitch, and/or roll between the sensors of the first device 122 and second device 124. The sensors may include, for example, an inertial measurement unit (IMU), accelerometer, gyroscope, one or more strain gauges, etc. The first device 122 and second device 124 may also include additional sensors or devices to obtain other data. For example, the first and second devices 122 and 124 may include an external temperature sensor to sense temperature of skin, one or more internal temperature sensors to sense temperature of one or more components of the sensor itself, a communication module, a Bluetooth Low Energy (BLE) module, visual indicators (e.g., light emitting diodes or LEDs), a magnetometer (to determine absolute movements and orientations of the patient), a compass, a barometer, etc. The first device 122 and second device 124 may also be configured to measure biometrics 1100 such as skin temperature, skin moisture, heartbeat, breathing rate, etc. The first device 122 and/or second device 124 may include quantum dots, optical sensors, etc. The first device 122 and/or second device 124 may be configured to remain coupled to the user's body for a day, two days, three days, four days, five days, six days, a week, two weeks, a month, or any other suitable time period.

The electronic circuitry in each of first device 122 and second device 123 may couple to at least one sensor. The electronic circuitry may be configured to control a measurement process and transmit measurement data, wirelessly or via one or more wires, to computer 21.

The first device 122 and second device 124 may include a power source (e.g., battery, capacitor, cell such as a Lithium-ion cell, etc.), energy harvesting devices, and/or may be configured to receive power from an external source or commercial supply device (e.g., via wired or wireless connection, such as with wireless transceivers). The electronic circuitry may include power management circuitry configured to receive energy by inductive coupling, light coupling, or radio frequency coupling that is harvested and stored in first device 122 and/or second device 124 until sufficient energy may be stored to power the first device 122 and/or second device 124 to complete a measurement.

The computer 21 may include one or more software programs to process measurement data received from first device 122 and/or second devices 124. The computer 21 may be any device having a processor, digital logic, a microprocessor, a microcontroller, a digital signal processor, a data conversion module, etc. that may be configured to support the software to process measurement data. For example, computer 21 may be a medical device, a phone, a tablet, a notebook computer, a personal computer, a robotic system, or a hand held device, among other examples. The computer 21 may have an application or “app” that is configured to direct a person through one or more movements to complete the process of registration for first device 122 and/or second device 124. The computer 21 may include visual, audible, or haptic feedback related to the registration process. A display of the computer 21 may provide visual feedback to support a person in real-time to complete the registration process, including instructions on how to perform the registration process as well as real-time feedback as the person performs the registration process.

Using data collected over time from the first device 122 and second device 124, a change of the knee angle over a time period such as a day may be determined. The processed and calibrated data from each device 122, 124 can be passed to an orientation estimation unit, which may determine orientations of the first device 122 and second device 124. In some implementations, the data and/or determinations made by the orientation estimation unit may include the pitch and roll undergone by the first device 122 and second device 124. Calculated parameters such as pitch and roll data can be passed to a transmitter, such as an orientation data packing unit, for onward transmission. Such onward transmission could be by wired connections or unwired connections such as Bluetooth transmission or radio transmission etc. The computer 21 may include a processor, data conversion unit, a calibration unit, an orientation estimation unit, and/or the orientation data packing unit to analyze and transmit the data collected from the first device 122 and the second device 124.

Referring to FIG. 9, wearable sensors 114 may also be implemented as a tracking system 130 for a chest and/or shoulder 92. The tracking system 130 may include one or more devices 132 provided, for example, on a chest, torso, arm, leg, or back (e.g., surrounding shoulder 92). In some embodiments, tracking system 130 may include just one device 132 with only one IMU. The devices 132 may include any of the features of devices 122, 124 described with reference to FIG. 8. As an alternative to a leg 62 and/or shoulder 92, tracking systems 120 and/or 130 may be used to measure orientations, angles, alignments, acceleration, forces, etc. of other anatomical portions of the body such as a torso or pelvis area. For example, the tracking systems 120 an/or 130 may be used to measure or determine strength and/or force such as push-off power at a toe during walking. The tracking systems 120 and/or 130 may be used to measure orientations, angles, alignments, etc. of other joints, such as a hip joint, ankle joint, neck joint, etc. The tracking systems 120 and/or 130 may be used over a period of time to analyze data and assess balance or stability as a patient performs daily activities and/or activities that are routine for their lifestyle.

### Preoperative Outputs 7000

Referring to FIGS. 1 and 3, the preoperative outputs 7000 may be determined via one or more preoperative algorithms 4000. The preoperative algorithms 4000 may also consider and/or analyze other previously stored data 50 of memory system 40 to determine preoperative outputs 7000. The preoperative outputs 7000 may include a prehabilitation plan 7010, a procedure, medical treatment, or surgical plan 7020, a postoperative plan 7030, a bone density score 7040, a fall risk or stability score 7050, a morphology score 7060, an EMG score 7070, an activity quality score 7080, a joint stiffness score 7090, a patient readiness score 7100, psychosocial score 7110, a b-score or bone shape score 7120, a push-off power score 7130, and a fracture risk score 7140. This list is not exhaustive, however. A “treatment course” or “course of treatment” may refer to any one of or all of the prehabilitation plan 7010, procedure plan 7020, and postoperative plan 7030 and/or their intraoperatively determined and postoperatively determined anologs described later.

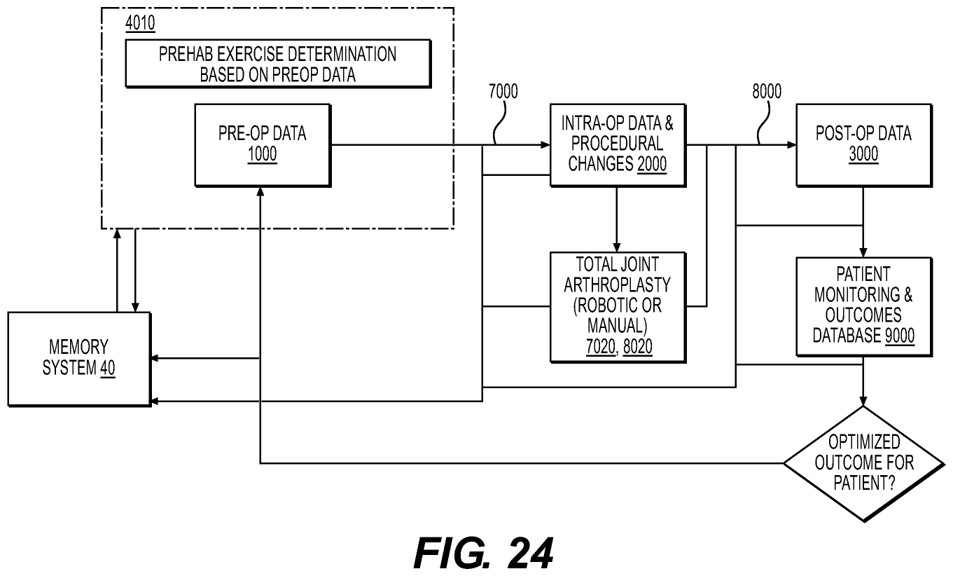

The prehabilitation plan 7010 may include instructions for a patient in preparing for a medical procedure or treatment course, such as surgery. For example, the prehabilitation plan 7010 may include an exercise program which may include, a type of an exercise, a length of the exercise, a frequency of the exercise, or an order of a plurality of exercises. The prehabilitation plan 7010 may include a priority order of muscles to strengthen, etc. in preparation for the procedure. The prehabilitation plan 7010 may include other instructions or plans, such as medicine information (e.g., dosage and type) for the patient to take before the procedure. The prehabilitation plan 7010 may be configured to reduce a recovery time after the procedure. The prehabilitation plan 7010 may be based on one or more other postoperative outputs 7000, such as the fall risk score 7050 and/or a stability score, bone density score 7040, activity quality score 7080, joint stiffness score 7090, patient readiness score 7100, psychosocial score 7110, b-score 7120, push-off power score 7130, fracture risk score 7140, etc. For example, patients with a higher fall risk score 7050, fracture risk score 7140, and/or a lower bone density score 7040 or push-off power score 7130 may need modified exercises. Specific embodiments of processes, algorithms, and/or feedback loops involving determinations by the preoperative algorithms 4000, intraoperative algorithms 5000, and postoperative algorithms 60000 will be described later with reference to FIGS. 22 and 24.

The procedure, medical treatment, or surgical plan 7020 may include instructions for a surgeon in preparing for and/or performing a procedure (e.g., surgery) on the patient. For example, when the procedure plan 7020 is a surgical plan 7020 for installation of an implant, the surgical plan 7020 may include, for example, a planned number, position, length, slope, angle, orientation, etc. of one or more tissue incisions or bone cuts, a planned type of the implant, a planned design (e.g., shape and material) of the implant, a planned or target position or alignment of the implant, a planned or target fit or tightness of the implant (e.g., based on gaps and/or ligament balance), a desired outcome (e.g., alignment of joints or bones, bone slopes such as tibial slopes, activity levels, or desired values for postoperative outputs 9000), a list of steps for the surgeon to perform, a list of tools that may be used, a planned operating room layout (e.g., positions and/or movement of objects or people in the operating room, such as staff, surgeons, medical or surgical robot 210, operating room table, patient, cameras, GUI 214, sensors, or other equipment), etc. The procedure plan 7020 may also include predictive or target outcomes and/or parameters, such as target postoperative range of motion and alignment parameters, target fall risk or fracture scores, activity quality scores, and joint stiffness scores. These target parameters may be compared (e.g., by patient expectations algorithm 4030 in FIG. 22) postoperatively to corresponding measured postoperative data 3000 and/or determined postoperative outputs 9000 to determine whether an optimized outcome for a patient was achieved.

A design of the implant may include, for example, curvatures, shapes, or thicknesses and/or shimming parameters corresponding to a patient's anatomy (e.g., from bone imaging data 1080). For example, a design of the implant and/or prosthetic may be configured to match an arc of curvature of the implant with an arc of curvature of the native femoral condyle of the patient, an arc or curvature of a socket area or acetabulum, an arc or curvature of a glenoid or humerus, an arc or curvature of a tibial condyle, etc. Aspects disclosed herein may be applied to a custom knee implant design, custom hip implant design, custom partial knee or hip implant design, or custom design of any other implant design for any other part of a patient's anatomy. The design of the implant may also include materials of the implant and/or placement of implants of autologous tissue, allograft tissue, and/or synthetic materials. The design of the implant may include thicknesses, a number of shims configured to be stacked and/or removed, a size of an added shim, or other dimensions configured to adjust a fit or tightness of the implant.

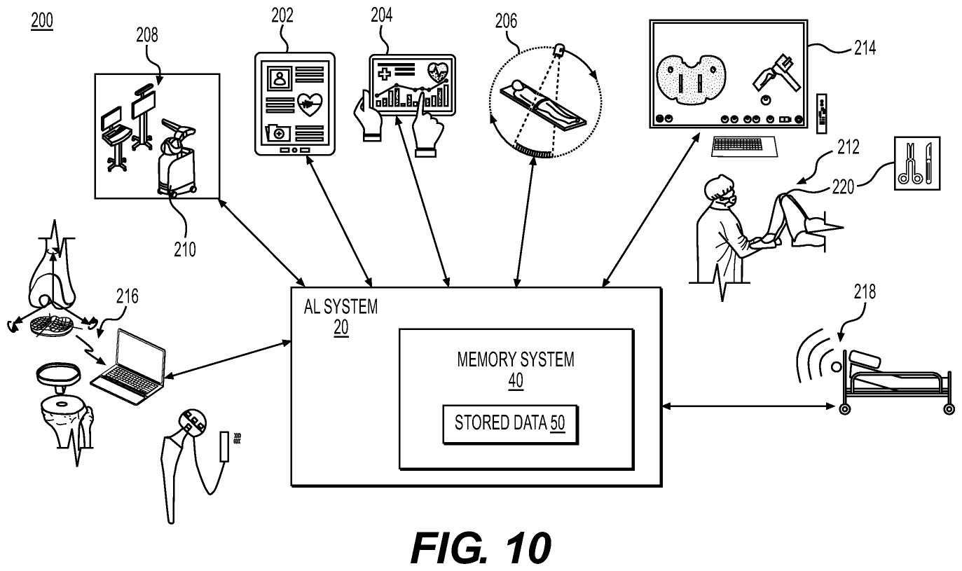

The procedure plan 7020 may also include instructions for a medical or surgical robot 210 to execute (see FIG. 10). Like the prehabilitation plan 7010, the procedure plan 7020 may be based on other preoperative outputs 7000. For example, in patients with a lower bone density score 7050 and a lower joint stiffness score 7090 (e.g., knee stiffness score), the procedure plan 7020 may include an alignment of a tibial prosthetic with a lower tibial slope and/or a lower number of incisions.

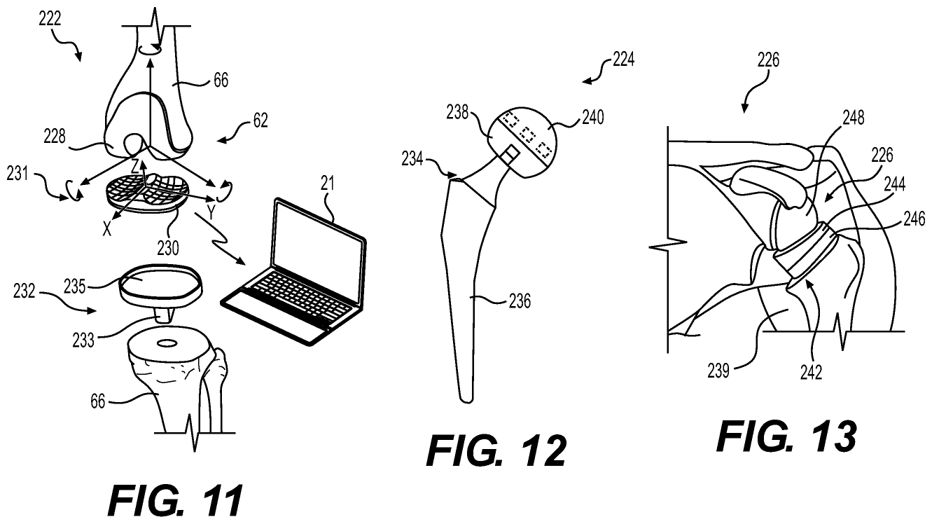

For example, with respect to FIG. 5 (see also FIG. 10), the procedure plan 7020 may include instructions on how to prepare a proximal end of the tibia 66 to receive a tibial implant 232 (FIG. 11), how to prepare a distal end of the femur 68 to receive a femoral implant 228 (FIG. 11), how to prepare a glenoid or humerus to receive a glenoid sphere 248 and/or humeral prosthetic component 242 (FIG. 13), how to prepare a socket area or acetabulum to receive a ball joint 238 (FIG. 12), etc. The bone surface may be cut, drilled, or shaved relative to a reference (e.g., a transepicondylar axis). Bone cuts or drills to the femur and tibia may also be made referenced to the vertical axis 84, mechanical axis 68, and/or anatomical axes 86, 90. The prepared bone surface may have a medial-lateral slope, anterior-posterior slope, and a compound slope configured to support accurate leg movement and proper rotation of the implant over a range of motion. The procedure plan 7020 may include positions lengths, and other dimensions for the surfaces and/or values for the slopes for bone preparation. As will be described later, the procedure plan 7020 may be updated and/or modified based on intraoperative information 2000.

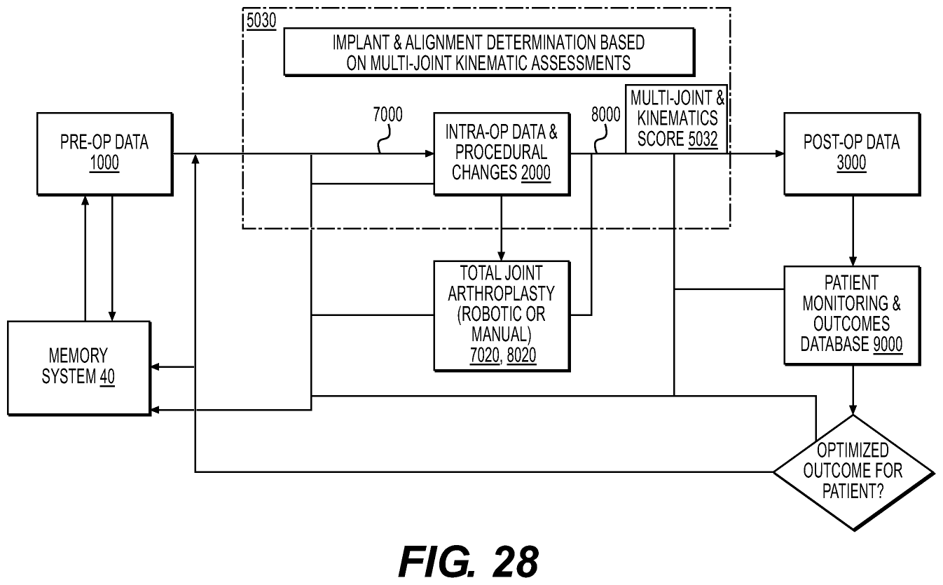

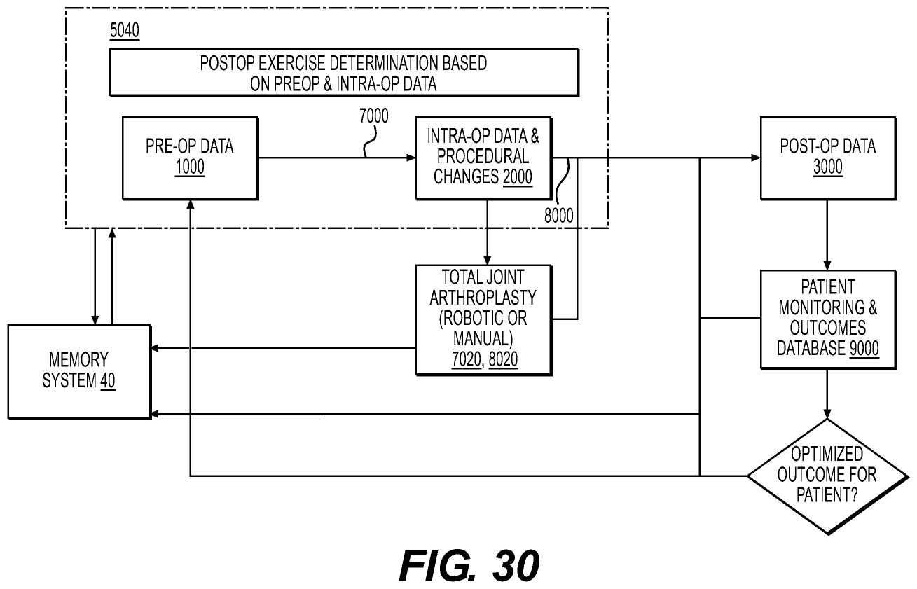

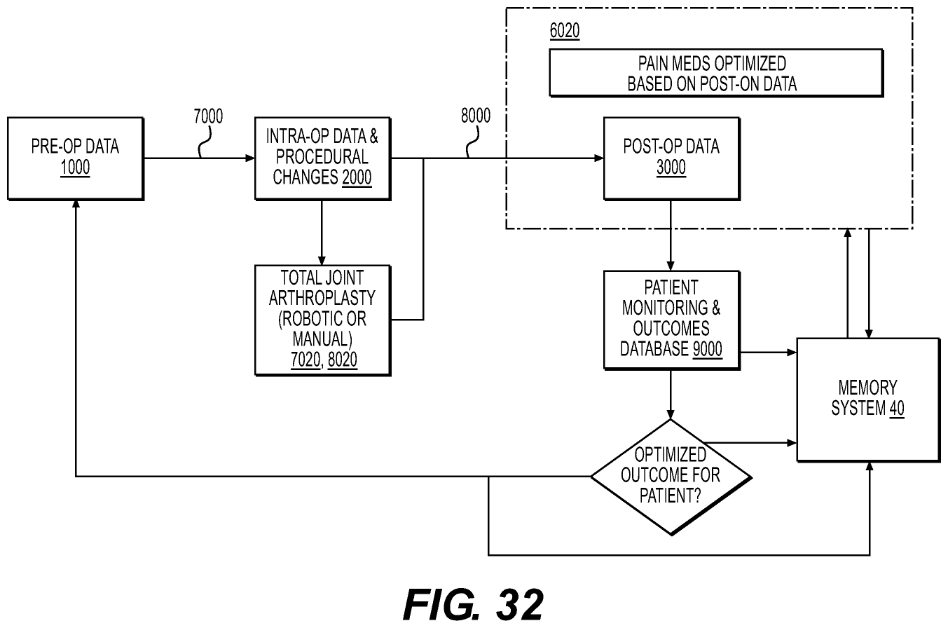

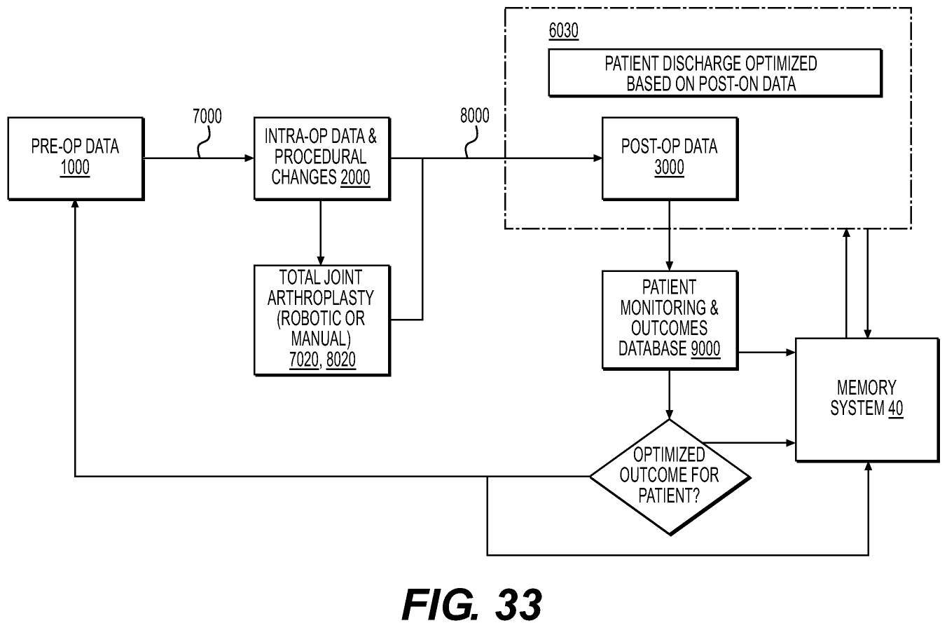

The postoperative plan 7030 may include plans similar to the prehabilitation plan 7010 such as an exercise program configured to decrease a recovery time of the patient. The postoperative plan 7030 may further include a medication plan (e.g., pain medication plan including a type, dosage, and/or tapering of pain medication) and/or a discharge plan including a length of stay in a hospital. The postoperative plan 7030 may include a schedule of follow-up visits with a practitioner, surgeon, physical therapist, etc. The postoperative plan 7030 may also include a plan for revision surgeries or future additional surgeries, though the procedure plan 7020 may be configured to reduce a likelihood of revision procedures or surgeries. Like the prehabilitation plan 7010 and procedure plan 7020, the postoperative plan 7030 may be based on other preoperative outputs 7000. For example, the postoperative plan 7030 may include an exercise program configured to target muscles based on the patient's lifestyle 1020 (e.g., frequency of climbing stairs or frequency of entering/exiting cars), the fall risk score 7050, and/or the fracture score 7140. Specific embodiments of determinations will be described later with reference to FIGS. 22-33. The procedure plan 7020 may be updated and/or modified based on intraoperative information 2000 and postoperative information 3000.

The bone density score 7040 may be calculated from bone density data 1090, bone imaging data 1080 (e.g., morphology/anthropometrics data 1082), medical history 1030, and/or other information input by a patient or practitioner. The bone density score 7040 may be implemented as a T-score where a higher score correlates to a greater bone density, but aspects disclosed herein are not limited.

The fall risk score 7050 may be calculated from kinematics 1110, range of motion 1112 (e.g., postural sway), and alignment 1114. The fall risk score 7050 may be paired with or be calculated based on lifestyle data 1020. For example, the fall risk score 7050 may be calculated on a mobile device 108, be updated based on information sensed by the mobile device 108, and be displayed on the mobile device 108 (e.g., in a fall risk tracking app). The fall risk score 7050 may also be based on other preoperative outputs 7000 and/or qualitative observations or scores (e.g., frailty based on walking patterns or walking patterns assessed based on height and/or weight) and/or other observations input by a practitioner or patient (e.g., using EMR 102 and/or interfaces 104). A higher fall risk score 7050 may indicate a higher likelihood that a patient will fall or lose balance, or a higher frailty of the patient, but aspects disclosed herein are not limited.

The morphology score 7060 and/or a b-score or bone shape score 7120 may be calculated from bone imaging data 1080 and morphology/anthropometrics data 1082 using, for example, statistical shape modelling (SSM) or other processes. The morphology score 7060 and/or a b-score 7120 may also account for other data, such as alignment 1114, fractures, etc. “Machine-learning, MM bone shape and important clinical outcomes in osteoarthritis: data from the Osteoarthritis Initiative” by Michael A. Bowes, Katherine Kacena, Oras A. Alabas, Alan D. Brett, Bright Dube, Neil Bodick, and Philip G. Conaghan, first published Nov. 19, 2020, explains details on calculating a b-score 7120 and is incorporated by reference herein in its entirety.

The EMG score 7070 may be based on EMG data 1040 and may indicate an activity level of neurons and/or muscles. A higher EMG score 7070 may correspond to a higher level of activity, but aspects disclosed herein are not limited. The activity quality score 7080 may be based on lifestyle 1020, medical history 1030, EMG data 1040 and/or the EMG score 7070, kinematics 1110, range of motion 1112, biometrics 1100, fitness level, and/or patient reported information. A higher activity quality score 7080 may indicate a higher activity level, activity quality, and/or fitness level of the patient, but aspects disclosed herein are not limited to a configuration or calculating of the activity quality score 7080.

The joint stiffness score 7090 may be calculated based on bone imaging 1080, kinematics 1110 (e.g., how quickly a patient can bend a joint), range of motion 1114, alignment 1114, etc. Each joint (e.g., knee, hip, ankle, neck) may have its own joint stiffness score 7090. A higher joint stiffness score 7090 may mean a higher stiffness and/or less laxity at the joint, but aspects disclosed herein are not limited.

The patient readiness score 7100 may be calculated based on psychosocial 1060 information (e.g., stress level) and/or the psychosocial score 7010, biometrics 1100 (e.g., sleeping patterns), kinematic 1110, bone imaging 1080 etc. to assess a readiness for surgery. The patient readiness score 7100 may be updated or modified based on kinematics 1110, etc. measuring during performance of the prehabilitation plan 7010, and the prehabilitation plan 7010 may be updated and/or modified based on updated to the patient readiness score 7100. As an example, biometrics 1100 indicated a decreased heart rate variability or HRV may indicate a higher level of stress and in turn a lower patient readiness score 7100.

The psychosocial score 7110 may be based on psychosocial 1060 information, such as stress, perceived pain, etc. and may also be based on biometrics 1100. The psychosocial 1060 information may be collected from surveys, practitioner observations, etc. A higher psychosocial score 7110 may indicate a higher level of stress, or alternatively may indicate a higher level of satisfaction, though aspects disclosed herein are not limited to a calculation of the psychosocial score 7110. A decreased HRB may indicate a higher level of stress and in turn a higher psychological score 7110. Alternatively, the psychosocial score 7110 may be configured to decrease based on a higher level of stress.

The push-off power score 7130 may be based on kinematics 1110, such as measured force, acceleration, contact pressure, etc. at a foot during walking (e.g., from a sensor in a shoe, coupled to the shoe, or coupled to the leg). A higher push-off power score 7130 may indicate a faster or stronger push-off during walking or spring in a step. Alternatively or in addition thereto, the push-off score 7130 may be measured at the hands, such as during push-ups.

The fracture risk score 7140 may be calculated from kinematics 1110, range of motion 1112 (e.g., postural sway), bone density 1090, and alignment 1114. The fracture risk score 7140 may be paired with or be calculated based on lifestyle data 1020 and/or the fall risk score 7050. For example, the fracture risk score 7140 may be calculated on a mobile device 108, be updated based on information sensed by the mobile device 108, and be displayed on the mobile device 108 (e.g., in a fracture risk tracking app). The fracture risk score 7140 may also be based on other preoperative outputs 7000 and/or qualitative observations or scores (e.g., frailty based on walking patterns or walking patterns assessed based on height and/or weight) and/or other observations input by a practitioner or patient (e.g., using EMR 102 and/or interfaces 104). As an example, a lower bone density score 7040 and a higher fall risk score 7050 may result in a higher determined fracture risk score 7140. A higher fracture risk score 7140 may indicate a higher likelihood that a patient will fracture a bone, or a higher frailty of the patient, but aspects disclosed herein are not limited.

### Intraoperative Data 2000

Referring to FIGS. 2, 3, and 10, the intraoperative data 2000 may include information taken during performance of a procedure plan 7020. The intraoperative data 2000 may include information on operating room efficiency 2010, procedure duration 2020, tourniquet time 2030, blood loss 2040, biometrics 2050, incision length 2060, soft tissue integrity 2070, pressure 2080, range of motion or other kinematics 2090, implant position or alignment 2100, and implant type or design 2110, though this list is not exhaustive. For example, intraoperative data 2000 may also include updated preoperative data 1000 (e.g., updated bone imaging 1080, etc.).

Operating room efficiency 2010 may include procedure duration information 2020, a number of practitioners performing the procedure plan 7020/8020, a number of medical or surgical tools used, etc. Operating room efficiency 2010 may also include information on an operating room layout, such as a room size, a setup, an orientation, starting location, and/or movement path of certain objects (e.g., surgical robot 210, practitioner, surgeon or other staff member, operating room table, cameras, GUI 214, other equipment, or patient). Cameras and/or a navigational system may be used to track operating room efficiency 2010 and/or layout information. Operating room efficiency 2010 may include information on staff and/or surgeon's performing the procedure plan 7020/8020, experience of each staff member or surgeon, past surgeries performed by each staff member or surgeon, and also scheduling information in an institution (e.g., hospital) where the surgery is taking place. Operating room efficiency 2010 may also include information on ergonomics for each staff member or surgeon, such as movement and posture patterns (measured by, for example, wearable sensors 114, external sensors, cameras and/or navigational systems, surgical robot 210, etc.) System 20 may make determinations to optimize operating room efficiency 2010. For example, based on ergonomics information, system 20 may determine that a table is too high for a surgeon and determine a lower height for the table in an updated operating room layout to include in the procedure plan 7020, which may increase operating room efficiency 2010 by reducing fatigue for a surgeon workingover the operating table.

Procedure duration 2020 may include duration and/or other timing data of certain steps or procedures of the procedure plan 7020 and/or a total time of the procedure plan 7020. Tourniquet time 2030 may include a time a tourniquet, cuff, or other restrictive device is applied to a limb. In addition, tourniquet time 2030 information may include pressure information at specific times or for specific time periods, where pressure information may be pressure applied to the limb, blood pressure, and/or pressure of, for example, an inflatable tourniquet. Blood loss 2040 may include information on an amount of blood lost during performance of the procedure plan 7020. Biometrics 2050 may include all types of information included in preoperative biometrics 1110 and may also include other patient characteristics, such as temperature, heart rate, breathing rate, skin temperature, skin moisture, pressure exerted on the patient's skin, patient movement/activity etc. during performance of the procedure plan 7020, etc. Incision length 2060 may include a length, position, and/or number of incisions actually made during performance of the procedure plan 7020. Actual incision length 2060 may correspond to or be different from a predicted or planned incision length from data in the planned procedure 1050 and/or procedure plan 7020.

Soft tissue integrity 2070 may include structural, strength, or density information for muscles, tendons, ligaments, and/or other soft tissue structures (e.g., skin) of the patient. Soft tissue integrity 2070 may be based on observed injuries (e.g., Posterior Cruciate Ligament or PCL injuries) during performance of the procedure plan 7020 and/or based on prior observations. Soft tissue integrity 2070 may be an input and/or an output based on other preoperative inputs 1000 and intraoperative inputs 2000. Soft tissue integrity 2070 may be determined from a laxity assessment where a physician may stress a joint to determine tissue integrity. The laxity assessment may be a manual and subjective process, or alternatively may be controlled and/or quantified with sensors (e.g., wearable sensors 114, sensored implants 216) to measure applied force and/or joint displacement. For example, a practitioner may perform a varus/valgus stress test on a knee where a controlled force is applied to a shank to assess collateral ligaments. Diagnostic imaging systems 106 such as MRI scans may also be used to assess tissue integrity and/or to reveal structural or physiological changes. As another example, a practitioner may use a pendulum knee drop test (passive test) to determine overall stiffness or knee joint laxity. Soft tissue integrity 2070 may also be determined from bone density 1090, which may be determined from diagnostic imaging systems 106, as bone density 1090 may be correlated to ligament integrity and/or soft tissue integrity 2070

Pressure 2080 may include information about a pressure or load (e.g., a contact pressure) applied to a patient's anatomy and/or a prosthetic component during performance of the procedure plan 7020. For example, pressure 2080 may include information on a magnitude and a position or center of a load applied to a prosthetic component or implant (e.g., humeral component, glenosphere component, tibia component, femoral component, etc.). Range of motion 2090 may include similar information as preoperative range of motion 1112, although a surgeon may be manipulating a patient's body instead of the patient manipulating his or her own body. Intraoperative range of motion 2090 may include manipulation under anesthesia (MUA) data based on movements, exercises, stretches, and/or other manipulation performed by the surgeon to assess movement, release pain, and break up scar tissue.

Implant position 2100 may include information on an actual implant position or alignment during performance of the procedure plan 7020. Actual implant position 2100 may correspond to or be different from a predicted or planned implant position from data in the planned procedure 1050 and/or the procedure plan 7020. Similarly, implant type 2100 may include information on an actual implant type, design, material, etc. during performance of the procedure plan 7020. Actual implant type 2100 may correspond to or be different from a predicted or planned implant type in the planned procedure 1050 and/or the procedure plan 7020. For example, a practitioner may record a different implant type 2100 used for a procedure that is different from the planned implant type 2100.

### Measurement System 200

Referring to FIGS. 2 and 10, the system 20 may collect intraoperative data using the intraoperative measurement system 200. Like the preoperative measurement system 100, the intraoperative measurement system 200 may include electronic medical records (EMR) 202, user interfaces or applications 204, and diagnostic imaging systems 206. The intraoperative measurement system 200 may also include a medical or surgical robotic system 208 including one or more robots 210, a sensored medical or surgical tool system 212, one or more sensored implants 216, and a sensored patient bed or operating table 218. EMR 202, user interfaces 204, diagnostic imaging systems 206, robotic system 208, robot 210, sensored tool system 212, motion sensor system 214, sensored implant 216, and sensored bed or table 218 of the intraoperative measurement system 200 may each include one or more communication modules (e.g., WiFi modules, BlueTooth modules, etc.) configured to transmit intraoperative data 2000 to the memory system 40, the system 20, to each other, etc.

The system 20 may use EMR 202 to collect the same types of information as with preoperative EMR 102, and EMR 202 may include any of the features of preoperative EMR 102 discussed hereinabove. EMR 202 may also include updated records including intraoperative observations by one or more practitioners performing the procedure plan 7020. The system 20 may use EMR 202 to collect and/or store operating room (OR) efficiency 2010, procedure duration 2020, tourniquet time 2030, blood loss 2040, biometrics 2050, incision length 2060, soft tissue integrity 2070, implant type 2110, etc.

The system 20 may implement user interfaces 204 on electronic devices such as computers, tablets, and/or phones, for example via mobile applications and/or management websites or interfaces such as OrthologIQ®, to display and/or update intraoperative data 2000 or other relevant data as received. User interfaces 204 may present questionnaires, surveys, or other prompts for practitioners to enter information, such as information to update EMR 202, pressure data 2080, etc. These user interfaces 204 may communicate with one or more of the other devices in the intraoperative measurement system 200 to display other data, such as pressure 2080 obtained from one or more pressure or load sensors (e.g. from the surgical robot system 208, the sensored surgical tool system 212, the sensored implants 216, and the sensored patient bed 218, etc.). User interfaces 204 may include graphical user interfaces (GUIs) 214 described in more detail later that may display intraoperative data 2000 and/or outputs 8000. These user interfaces 204 may be executed on other devices disclosed herein (e.g., using mobile devices or other computers).

Diagnostic imaging systems 206 may include computed tomography (CT) scans, magnetic resonance imaging (MM), x-rays, etc. For example, just prior to starting a procedure and/or during performing the procedure plan 7020, a fluorescence imaging system or other non-invasive imaging system may capture images of a patient's anatomy and update, in real time, these images (e.g., by displaying these images via GUI 214). Diagnostic imaging systems 206 may be used to collect and/or update, intraoperatively, bone imaging information 1080, including morphology and/or anthropometrics 1082 fractures, and bone density 1090.

The surgical robotic system 208 may include one or more surgical robots 210 configured to perform or assist with, via automated movement and/or sensing, at least a portion of the procedure plan 7020. The surgical robot 210 may be implemented as or include one or more automated or robotic surgical tools, robotic surgical or Computerized Numerical Control (CNC) robots, surgical haptic robots, surgical tele-operative robots, surgical hand-held robots, or any other surgical robot. The surgical robot 210 may include or be configured to hold (e.g., via a robotic arm), move, and/or manipulate surgical tools and/or robotic tools such as cutting devices or blades, jigs, burrs, scalpels, scissors, knives, implants, prosthetics, etc. The surgical robot 210 may be configured to move a robotic arm, cut tissue, cut bone, prepare tissue or bone for surgery, and/or be guided by a practitioner via the robotic arm to execute a procedure plan 7020,

The surgical robot 210 may include sensors (e.g., pressure sensors, temperature sensors, load sensors, strain gauge sensors, force sensors, weight sensors, current sensors, voltage sensors, position sensors, IMUs, accelerometers, gyroscopes, position sensors, optical sensors, light sensors, ultrasonic sensors, acoustic sensors, infrared or IR sensors, cameras, etc.) on one or more robotic arms, robotic tools or devices, or surgical tools; and may collect data during performance of the procedure plan 7020 such as procedure duration 2020, biometrics 2050, pressure 2080, incision length 2060, implant position 2100, and/or implant position 2100. Data collected from the surgical robot 210 may be referred to as robotic data.

The surgical robot 210 may include one or more wheels to move in an operating room, and may include one or more motors configured to spin the wheels and also manipulate surgical limbs (e.g., robotic arm, robotic hand, etc.) to manipulate surgical or robotic tools or sensors. The surgical robot 210 may be a Mako SmartRobotics™ surgical robot, a ROBODOC® surgical robot, etc. However, aspects disclosed herein are not limited to mobile surgical robots 210.

The surgical robot 210 may be controlled automatically and/or manually (e.g., via a remote control or physical movement of the surgical robot 210 or robotic arm by a practitioner). For example, the procedure plan 7020 may include instructions that a processor, computer, etc. of the surgical robot 210 is configured to execute. The surgical robot 210 may use machine vision (MV) technology for process control and/or guidance. The surgical robot 210 may have one or more communication modules (WiFi module, BlueTooth module, NFC, etc.) and may receive updates to the procedure plan 7020 and/or a new intraoperative procedure plan 8020 (described later with intraoperative outputs 8000). Alternatively or in addition thereto, the surgical robot 210 may be configured to update the procedure plan 7020 and/or generate a new intraoperative procedure plan 8020 for execution.

The sensored surgical tool system 212 may include one or more sensored surgical tools 220 (e.g., a sensored marker). The sensored surgical tool 220 may be applied to or be worn by the patient during the procedure plan 7020, such as a wearable sensor (e.g., wearable sensors 114), a surgical marker, a temporary surgical implant, etc. Although some surgical tools 220 may also be sensored implants 216 or surgical robots 210, other surgical tools 220 may not strictly be considered an implant or a robotic or automated device. For example, the sensored surgical tool 220 may also be or include a tool (e.g., probe, knife, burr, etc.) used by medical personnel and including one or more optical sensors, load sensors, load cells, strain gauge sensors, weight sensors, force sensors, temperature sensors, pressure sensors, etc. The system 20 may use the sensored surgical tool system 212 to collect data on pressure 2080, range of motion 2090, incision length 2060 and/or position, soft tissue integrity 2070, biometrics 2050, etc. The sensored surgical tool 220 may be or include a robotic handheld tool configured to be held in the surgeon's hand and automatically cut tissue or bone (and/or prepare tissue or bone for surgery) according to instructions from the procedure plan 7020. For example, the sensored surgical tool 220 may be or include a robotic burr, knife, or blade. The surgeon may hold a handle of the sensored surgical tool 220, and the sensored surgical tool 220 may execute instructions using feedback from sensors (e.g., for position and/or orientation) and using moveable or motorized tool heads (e.g., blade or knife head).

The one or more sensored implants 216 may include temporary or trial implants applied during the procedure plan 7020 and removed from the patient during the surgical procedure, and/or permanent implants 216 configured to remain for postoperative use. The sensored implants 216 may include one or more load sensors, load cells, force sensors, weight sensors, current sensors, voltage sensors, position sensors, IMUs, accelerometers, gyroscopes, optical sensors, light sensors, ultrasonic sensors, acoustic sensors, infrared or IR sensors, cameras, pressure sensors, temperature sensors, etc. The system 20 may use sensored implants 216 to collect data on range of motion 1112 (e.g., when the patient is manipulated by the surgeon during the procedure plan 7020), biometrics 2050, pressure 2080, implant position 2100 (e.g., alignment), implant type 2110 (e.g., design, material), etc. The one or more sensored implants 216 may also be configured to monitor infection information. More details on sensored implants 216 are provided with reference to FIGS. 11-13.

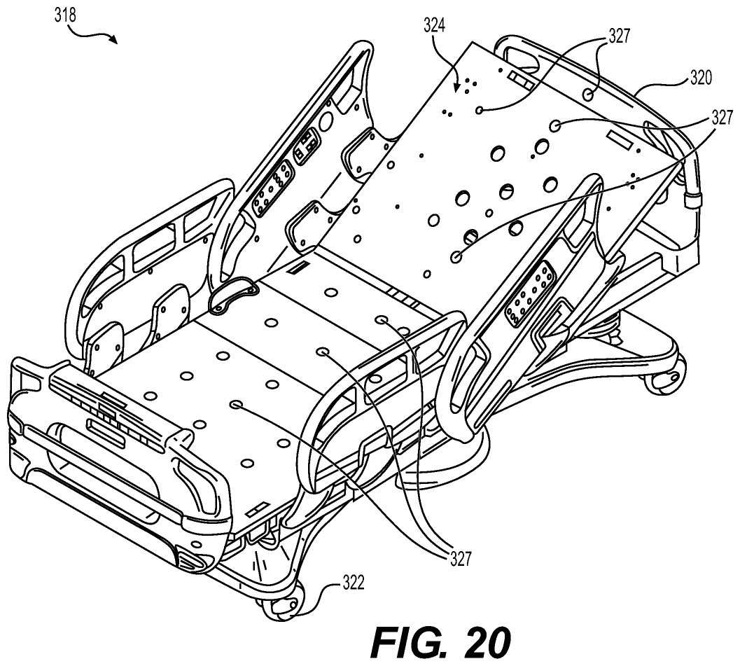

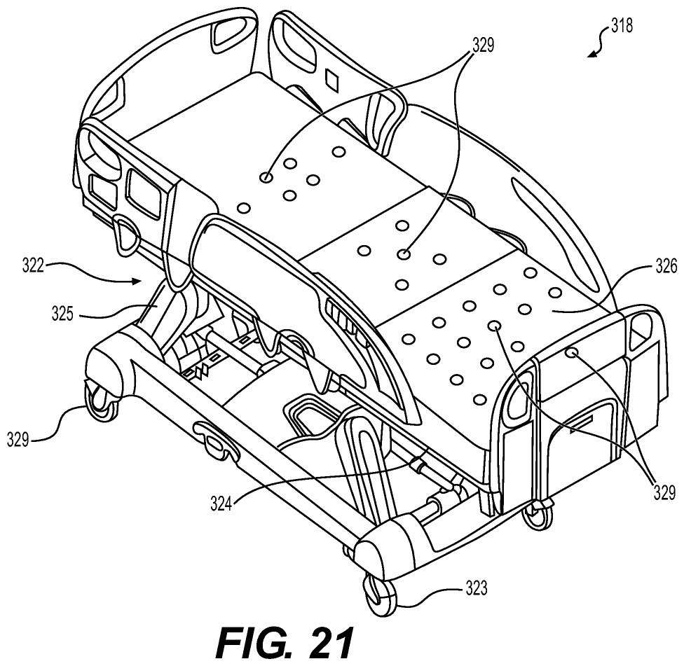

The one or more sensored patient bed or operating table 218 may be a bed or table including temperature sensors, load cells, pressure sensors, position sensors, accelerometers, IMUs, etc. The system 20 may use the sensored bed or table 218 to collect information on an orientation or position of the patient and biometrics 2050 (heart rate, breathing rate, skin temperature, skin moisture, pressure exerted on the patient's skin, patient movement/activity, etc.). The sensored bed or table 218 may include one or more wheels for movement, and the sensored bed or table 218 may collect information on movement of the bed or table 218, procedure duration 2020, etc. The system 20 may implement the sensored bed or table 218 as a postoperative sensored discharge bed 318 to sense patient movement and/or entrance/exit data. The postoperative sensored hospital or discharge bed 318 is described in more detail later with reference to FIGS. 20-21, and the sensored bed or table 218 may have a same or similar structure as the postoperative sensored discharge bed 318.

Referring to FIGS. 11-13, the one or more sensored implants 216 may be implemented as a knee prosthetic system 222, a hip prosthetic system 224, and/or a shoulder prosthetic system 226. Aspects disclosed herein are not limited to these types of knee, hip, and shoulder prosthetic systems 222, 224, 226. The one or more sensored implants 216 may be implemented as another implant system for another joint or other part of a musculoskeletal system (e.g., hip, knee, spine, bone, ankle, wrist, fingers, hand, toes, or elbow) and/or as sensors configured to be implanted directly into a patient's tissue, bone, muscle, ligaments, etc. Each of the knee, hip, and/or shoulder prosthetic systems 226 may include sensors such as inertial measurement units, strain gauges, accelerometers, ultrasonic or acoustic sensors, etc. configured to measure position, speed, acceleration, orientation, range of motion, etc. In addition, each of the knee, hip, and/or shoulder prosthetic systems 226 may include sensors that detect changes (e.g., color change, pH change, etc.) in synovial fluid, blood glucose, temperature, or other biometrics and/or may include electrodes that detect current information, ultrasonic or infrared sensors that detect other nearby structures, etc. to detect an infection, invasion, nearby tumor, etc. In some examples, each of the knee, hip, and/or shoulder prosthetic systems 226 may include a transmissive region, such as a transparent window on the exterior surface of the prosthetic system, configured to allow radiofrequency energy to pass through the transmissive region.

The prosthetic knee system 222 may include a femoral prosthetic component 228 configured to be coupled to a distal end of a femur 64, an insert 230, e.g., an alignment measurement device, and/or a tibial prosthetic component 232 configured to be coupled to a proximal end of a tibia 66. The femoral prosthetic component 228 may have one or more condyle surfaces (e.g., two condyle surfaces to mimic a natural femur). A design of the tibial prosthetic component 232 may include predetermined numbers, sizes, shapes, materials (e.g., metal or metal alloy) of the condyle surfaces and the femoral prosthetic component 228 as a whole. The insert 230 may be used to support installation of the femoral prosthetic component 230 and/or the tibial prosthetic component 232. The tibial prosthetic component 232 may include a tibial tray 235 and a tibial stem 233. A design of the tibial prosthetic component 232 may include predetermined sizes (e.g., lengths), shapes, materials (e.g., metal or metal alloy) of the tibial tray 235 and tibial stem 233. The tibial tray 235 may be configured to support and retain insert 230 to the tibial prosthetic component 232, and the tibial stem 233 may be configured to be inserted into a drilled hole in the tibia. For example, the tibial stem 233 may be configured to extend within the medullary canal of the tibia. Femoral prosthetic component 228 and/or tibial prosthetic components 232 may have one or more retaining features to couple to prepared bone surfaces of the femur 64 and/or tibia 66, respectively.

The femoral prosthetic component 228, the insert 230, and/or the tibial prosthetic component 232 may include one or more sensors (e.g., within the tibial tray 235 or within the tibial stem 233) configured to measure kinematics and/or range of motion 2090 such as position, speed, acceleration, orientation, load, pressure 2080, force, and/or other parameters (e.g., biometrics 2050 such as temperature, pulse, blood pressure, bone density, colors or changes to synovial fluid to detect infection related data, blood glucose, heart rate variability, sleep disturbances, etc.). Alternatively or in addition thereto, the prosthetic knee system 222 may include one or more sensors coupled directly to the femur 64 and/or tibia 66. In some examples, sensors positioned outside tibial prosthetic component 232, femoral prosthetic component 228, and insert 230 may be coupled to femur 64 and/or tibia 66, and may be in communication with electronic components within tibial prosthetic component 232, femoral prosthetic component 228, and/or insert 230.

The femoral prosthetic component 228, the insert 230, and/or the tibial prosthetic component 232 may include one or more inertial measurement units (IMU). For example, the tibial stem 233 of the tibial prosthetic component 232 may include a space or cavity to house one or more inertial measurement units (IMU). Alternatively or in addition thereto, the prosthetic knee system 222 may include an IMU coupled directly to the femur 64 and/or tibia 66. The IMU may support real-time alignment measurement. The IMU may measure alignment of a leg 62 and support changes or modifications prior to final installation of the femoral prosthetic component 228, the insert 230, and/or the tibial prosthetic component 232 to ensure alignment may be within a predetermined range for optimal performance and reliability.

The IMU may include three gyroscopes and three accelerometers, where a first, second, and third gyroscope and a first, second, and third accelerometer are respectively aligned to three perpendicular axes 231. Each gyroscope may measure an angular velocity corresponding to a rotation about an axis. In other examples, the IMU may include any number of gyroscopes and any number of accelerometers, may only include one or more gyroscopes and not include accelerometers, or may only include one or more accelerometers and not include gyroscopes. Each accelerometer may measure a change in motion (acceleration) corresponding to one of the axes. The IMU may include up to nine degrees of freedom (DOF), which may include accelerations, gyroscopic velocities, and magnetometer values for 3-dimensional space. For example, the IMU may include up to 9-DOF, 6-DOF, or 3-DOF, and is not limited to the above-described arrangement.

The IMU may include a micro-electro mechanical (MEMs) integrated circuit. For example, one or more of the gyroscopes or accelerometers may be or include a MEMs integrated circuit. A form factor of a MEMs gyroscope integrated circuit or MEMs accelerometer integrated circuit may support placement in a prosthetic component or coupling to a prosthetic component or bone surface to measure alignment of the muscular-skeletal system. The MEMs gyroscope may have a resonating mass that shifts with angular velocity and output a signal corresponding to (e.g., proportional to) the angular velocity of the IMU. A MEMs accelerometer may have a mass-spring system that shifts in response to an exerted acceleration, e.g., counter to a bias of a spring in the mass-spring system.

The femoral prosthetic component 228, the insert 230, and/or the tibial prosthetic component 232 may include other sensors, such as strain gauge sensors, optical sensors, pressure sensors, load cells/sensors, ultrasonic sensors, acoustic sensors, resistive sensors including an electrical transducer to convert a mechanical measurement or response (e.g., displacement) to an electrical signal, and/or sensors configured to sense synovial fluid, blood glucose, heart rate variability, sleep disturbances, and/or to detect an infection in the leg 62 and/or around the knee. Measurement data from the IMU and/or other sensors may be transmitted to a computer or other device of the system 20 to process and/or display alignment, range of motion, and/or other information from the IMU. For example, measurement data from the IMU and/or other sensors may be transmitted wirelessly to a computer or other electronic device outside the body of the patient to be processed (e.g. via one or more algorithms) and displayed on an electronic display.

The hip prosthetic system 224 may include a femoral prosthetic device or component 234 including a femoral stem 236 configured to couple to a femur 64 (FIG. 10) of a patient and a ball joint or head 238 configured to couple to a hip bone. A design the of the femoral prosthetic device or component 234 may include a size (e.g., radius), shape, material, radius, etc. of the femoral stem 236, ball joint 238 and/or a neck coupling the stem 236 to the ball joint 238. The hip prosthetic system 224 may have any of the features of the knee prosthetic system 222 described with reference to FIG. 11.

The femoral prosthetic device 234 may include (e.g., within the ball joint 238 and/or the stem 236) one or more sensors 240 such as strain gauge sensors, IMUs, optical sensors, pressure sensors, load cells, ultrasonic sensors, acoustic sensors, and/or sensors configured to sense synovial fluid and/or detect an infection (e.g., via blood glucose, body temperature, sleep disturbances, heart rate variability, etc.). The femoral prosthetic device 234 may be configured to measure, via the one or more sensors 240, magnitude, location, and/or direction of forces placed on the femoral prosthetic device 234 (e.g., on ball joint 238) and/or a position, orientation, speed, acceleration, etc. of the femoral prosthetic device 234 (e.g., ball joint 238).

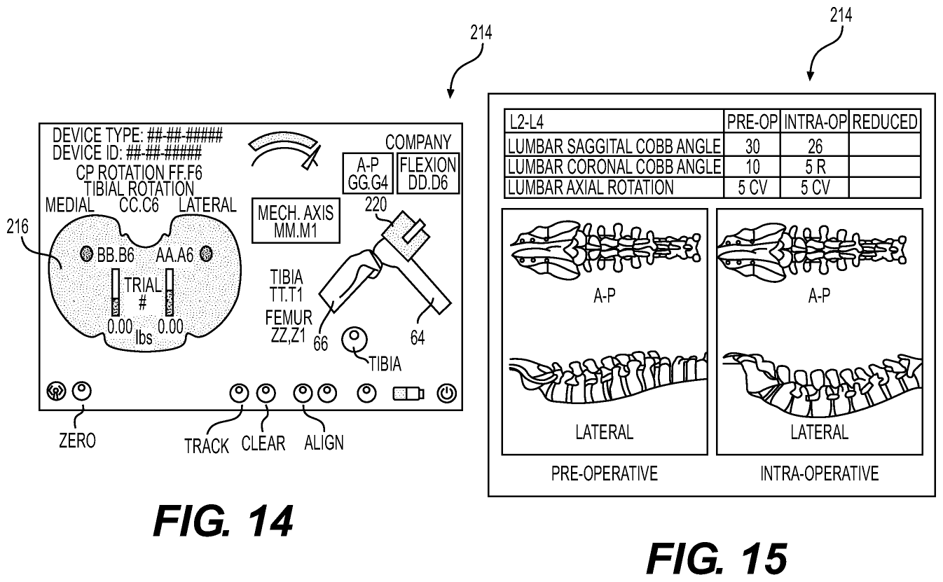

For example, the one or more sensors 240 may include three strain gauge sensors positioned circumferentially around a central circuit board of the ball joint 238 and positioned at an equal distance from a center of the ball joint 238 and/or some other reference. Each strain gauge sensor may be spaced equally from each adjacent sensor. Different strains, loads, pressures, forces, etc. measured by each strain gauge sensor may be processed to determine a load magnitude and location of the load applied to the ball joint 238. Alternatively or in addition thereto, the femoral stem 236 may include sensors to measure, for example, rotation of the femur about the hip joint and/or ball joint 238 and/or whether the femoral stem 236 is moving (e.g. loosely coupled to the femur, etc.). The measured strains and/or other data may be transmitted to the system 20 or another computing platform to calculate load parameters, such as magnitude, location, direction, etc. of an applied load, force, etc. of a joint (e.g., hip joint) in real time, which may then be visualized on a display (e.g., via GUI 214 described with reference to FIGS. 14-18).

Referring to FIG. 13, the shoulder prosthetic system 226 may include a glenoid prosthetic component or glenoid sphere 248, a humeral prosthetic component 242, and a measurement device or insert 244. The glenoid sphere 248 may be configured to be coupled to a prepared bone surface of a scapula 239, such as within the glenoid cavity 241 of the scapula 239. The glenoid sphere 248 may have an anchor or stem to support an attachment (e.g., via screws) to scapula 140. The glenoid sphere 248 has an external, convex curved surface configure to couple to the measurement device 244. The humeral prosthetic component 242 may be configured to couple to a prepared bone surface of a humerus 243. The humeral prosthetic component 242 may have a humeral tray 246 configured to couple with the measurement device 244. The measurement device 244 may have an external, concave curved surface configured to couple to the external, convex curved surface of the glenoid sphere 248.

The glenoid sphere 248, humeral prosthetic component 242, and/or measurement device 244 may include at least one sensor such as strain gauge sensors, IMUs, optical sensors, pressure sensors, load cells, ultrasonic sensors, acoustic sensors, and/or sensors configured to sense synovial fluid and/or detect an infection (e.g., via blood glucose, body temperature, sleep disturbances, heart rate variability, etc.). The one or more sensors may be configured to measure, via the one or more sensors, magnitude, location, and direction of forces placed on the glenoid sphere 248, humeral prosthetic component 242, and/or measurement device 244 and/or a position, orientation, speed, acceleration, etc. of the glenoid sphere 248, humeral prosthetic component 242, and/or measurement device 244.

As an example, a plurality of sensors (e.g., strain gauge sensors, capacitors and/or capacitive sensors, or IMUs) may be provided along a concave surface 245 of the measurement device 244 which is contact with the convex surface of the glenoid sphere 248. Alternatively or in addition thereto, the sensors and/or contact surfaces of the sensors may be raised (e.g., by 0.10 mm, 1 mm, 10 mm, etc.) with respect to a remaining portion of the concave surface 245 such that the glenoid sphere 248 contacts the measurement device 244 only at the raised contact surfaces of the sensors. The measurement device 244 may include electronic circuitry configured to control a measurement process and transmit measurement data to the memory system 40 and/or system 20 to be displayed on the GUI 214. The shoulder joint may be taken through a range of motion, and the sensors in the measurement device 244 may measure the range of motion 2090. For example, a position of humerus 243, a load magnitude applied to measurement device 244 by glenoid sphere 248, and/or a contact point where glenoid sphere 248 couples to measurement device 244 can be measured and/or determined in real-time.

Although prosthetics are described with reference to FIGS. 11-13, the sensored implants and/216 may also be implemented as implantable navigation systems. For example, the sensored implant 216 may have primarily a sensing function rather than a joint replacement function. The sensored implant 216 may, for example, be a sensor or other measurement device configured to be drilled into a bone, another implant, or otherwise implanted in the patient's body.

Intraoperative Outputs 8000

Referring to FIGS. 1 and 3, the intraoperative outputs 8000 may be determined via one or more intraoperative algorithms 5000. The intraoperative algorithms 5000 may also consider preoperative information 1000 and/or outputs 7000 and/or other previously stored data 50 of memory system 40 to determine intraoperative outputs 8000. The intraoperative outputs 8000 may include an updated or new procedure plan 8020, an updated or new postoperative plan 8030, an updated or new bone density score 8040, an updated or new fall risk or stability score 8050, an updated or new activity quality score 8060, an updated or new joint stiffness score 8070, a patient readiness score 8080, an updated or new B-score 8100, and an updated or new fracture risk score 8140. This list is not exhaustive, however. For example, the intraoperative outputs 8000 may also include some of the preoperative information 7000 previously described.

As previously described herein, intraoperative algorithms 5000 may be used to generate and output the procedure plan 8020. This procedure plan 8020 may be newly generated based on intraoperative information 8000 and/or may be a modification to the procedure plan 7020 generated using the preoperative information 7000 (and/or a manually input procedure plan 7020). For example, the intraoperative algorithm 5000 may determine that only minor changes are necessary to update the procedure plan 8020 based on range of motion 2090, biometrics 2050, actual incision length 2060 and/or implant position 2100 or type 2110, etc. As another example, a medical condition not known to a surgeon may not be apparent until intraoperative information 8000 is collected and analyzed (e.g., blood loss 2040, soft tissue integrity 2070, range of motion 2090, undetected bone fractures, etc.), and the intraoperative algorithm 5000 may generate a new procedure plan 8020 accounting for the detected condition. The procedure plan 8020 may include the same types of information and/or parameters as the preoperatively determined procedure plan 7020 (e.g., instructions on incisions, prosthetic type, etc.).

Referring to FIGS. 1, 3, and 14-18, during performance of the procedure plan 7020 and/or 8020, GUI 214 may display intraoperative data 2000 and/or intraoperative outputs 8000 quantitatively, as graphs and/or tables, schematically, and/or visually as illustrations, animations, and/or videos. For example, the GUI 214 may include or be implemented as GUI 214A, GUI 214B, 214C, 214D, or 214E, as shown in FIGS. 14-18, respectively. The GUI 214 (e.g., GUI 214A, 214B, and 214D) may be configured to visualize or illustrate bones (e.g., femur 64 and/or tibia 66, humerus, scapula, hip joint, ankle joint, spine, etc.), prosthetic components or implants (e.g., sensored prosthetics and/or implants 216), and/or surgical tools 220 (e.g., markers) currently applied to and/or interacting with the patient's anatomy.

The GUI 214 may also be configured to visualize (e.g., as a video, a virtual reality or VR platform, an augmented reality or AR platform, or a mixed reality or MR platform) real-time intraoperative data 2000 as its collected, such as range of motion 2090 from the prosthetics and/or implants 216 and/or surgical tools 220 (e.g., as in 214A), alignment, positions, and/or orientations of the prosthetics and/or implants 216, etc. (e.g., as in 214A, 214B, 214C, 214D, and 214E). The GUI 214 may be configured to display the real-time intraoperative data 2000 in multiple dimensions, such as 2D or 3D, and/or viewed with different mediums (e.g., a VR headset, an AR headset, or an MR headset) but not limited to the described devices. The GUI 214 may be interactive so that a surgeon or other staff member may interact with displayed data in real-time intraoperatively.

In some embodiments, GUI 214 may be configured to display an optimized outcome of an alignment of prosthetics and/or implants 216 included in the procedure plan 7020 (e.g., as in 214B or 214D), an updated optimized outcome included in the procedure plan 8020 based on intraoperative data (e.g., as in 214B or 214D), bone shape (e.g., spine shape) (e.g., as in 214B or 214D), etc., and/or a real-time actual alignment, position, etc. on a same electronic screen to facilitate comparison (e.g., as in 214B). The GUI 214 may be configured to display instructions, progress, and/or next steps of the procedure plan 7020 and/or 8020, and any alerts or warnings based on certain determinations from the intraoperative data 2000 and/or intraoperative outputs 8000 (low heart rate, high blood loss, etc.) and/or preprogrammed alerts or warnings (e.g., timing data, etc.) In some aspects, GUI 214 may display a determined operating room layout, schedule of medical personnel, workflows, etc. Any of the exemplified GUI 214A, GUI 214B, GUI 214C, GUI 214D, and/or GUI 214E may be displayed on an electronic screen, via one or more of the electronic devices discussed herein (e.g. a computer screen, mobile phone, tablet, surgical robot, etc.), either separately or at the same time as each other. GUI 214A, GUI 214B, GUI 214C, GUI 214D, and/or GUI 214E may be part of a surgical robotic system 208, part of a surgical robot 210, part of an operating room (OR) layout, part of a computer 21, etc.

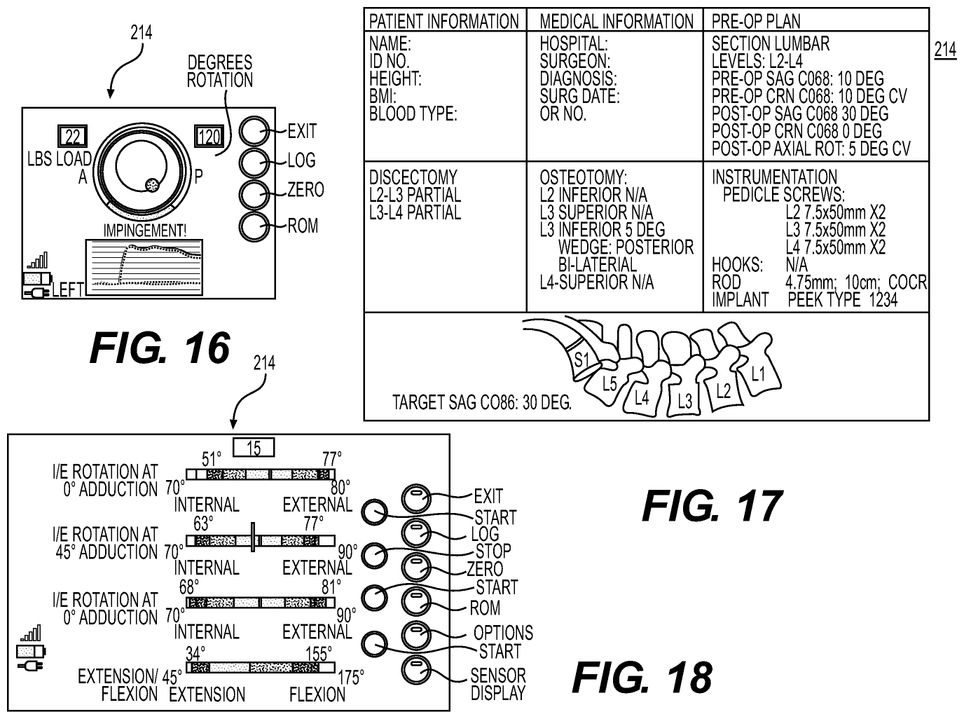

The GUI 214 may facilitate practitioners and/or an operating team in rapidly assimilating data to verify, adjust, or make changes that improve surgery (e.g., installation of an implant). For example, practitioners may make adjustments to a tension of different soft tissues, which may affect range of motion of a joint. As another example, GUI 214C may display impingement data, contact points of, and/or loads on an implant (e.g., FIG. 16). Practitioners and/or intraoperative or postoperative algorithms 5000 and/or 6000 may detect notching (e.g., scapular notching) from repetitive contact between a prosthetic and bone that causes an osteolytic or inflammatory reaction, and adjustments to a patient's anatomy may be determined or recommended (e.g., by postoperative algorithms 5000 and/or 6000 when determining or updating the procedure plan 7020 and/or 8020) when impingement or an impingement angle is detected to prevent notching from occurring. Displayed range of motion 2090 and other data may enable intraoperative and/or postoperative algorithms 5000 and/or 6000 to determine whether and/or how to make various adjustments to reduce or eliminate impingement, create more stability in a joint (e.g., by increasing thickness, a number of shims and/or a size of a shim in the implant, choosing an implant that results in certain geometries (e.g., shallower tibial slope), choosing a stabilizing implant, or configuring bone cuts or bone surfaces), increase a range of motion of the joint, reduce implant or prosthetic malpositioning, and/or adjust muscle tension. Actual adjustments made in surgery (e.g., by a practitioner or surgical robot 210) may be sensed, recorded, and input into the memory system 40 and/or used to update procedure plan 7020 and/or 8020.

As exemplified in FIG. 14, the GUI 214A may display data related to knee surgery. The relevant and/or recognized bones, such as the femur 64 and tibia 66, may be displayed. In addition, a sensored implant 216 (e.g., a tibial trial implant 216) installed intraoperatively may be displayed, along with information on the implant 216 such as a device ID, device type, weight, and other dimensions. The sensored implant 216 may be a trial insert, and the GUI 214A may display associated angular readings (e.g., CP Rotation, Mechanical Axis angle, Tibia angle, AP angle, Flexion angle, and Tray rotation), and loadings. The GUI 214A may display target angles and/or loads along with actual angles and/or loads. The target angles and/or loads may update in real time as the intraoperative algorithms 5000 make determinations that update or change the procedure plan 7020 and/or 8020 during the procedure, while the actual angles and/or loads may be updated based on detections from the trial insert 216 and/or other data sensed intraoperatively (e.g., from a surgical robotic system 208, from the practitioner, from a surgical tool system 212, etc.). As the actual and target values converge, the GUI 214A may display indicators (e.g., changing colors).

As another example, the sensored implant 216 may be or include a sensored implant installed in an instrumental cut guide (e.g., cutting jig) or other surgical tool 220, and targets relating to bone cuts (e.g., angles of a cut, position, depth, slope, etc.) may be displayed and/or adjusted (e.g., as determined by intraoperative algorithms 5000) as the practitioner makes cuts and/or as an offset (e.g., tibia offset, femur offset, varus or valgus offset) is sensed intraoperative (e.g., from surgical tool system 212, sensored implant 216, from the surgical robot system 208, from the practitioner, from diagnostic imaging system 206, etc.). The GUI 214A may display information from a tibial trial 216, instrumental cut guide or other surgical tool 220, or a combination of devices. The GUI 214A may show alignment data, cut data, or other data relative to a mechanical axis, tibia axis, femur axis, etc. This data may be collected intraoperatively or just prior to surgery as the practitioner moves the patient's body and/or limbs (e.g., leg) through a range of motion (prior to or after installing a temporary or trial implant 216), and may also be collected during installation of a permanent implant 216. FIG. 18 exemplifies that the GUI 214E may display quantitative data as the practitioner moves the patient's body through the range of motion (e.g., between flexion and extension of a joint, such as a knee, hip, or shoulder).

As exemplified in FIG. 15, the GUI 214B may display bones which were identified and/or recognized intraoperatively based on bone landmarks, such as each specific vertebra in a spine. The GUI 214B may display angles between recognized bones, such as a Cobb angle or an angle between a tibia and a femur, based on bone landmarks and/or other data. The GUI 214B may display target alignment data and/or images determined preoperatively (e.g., by preoperative algorithms 4000) in the procedure plan 7020 and/or updated intraoperatively by intraoperative algorithms 5000 in the procedure plan 8020. The GUI 214B may further display actual data and/or images on a same screen or display for comparison with the target images and/or data. In addition, the GUI 214B may display actual intraoperative images from before installation of an implant 216, and also after initial installation of an implant 216 for comparison with both the immediately prior intraoperative image (from before installation) and also the target alignment. The GUI 214B may be an interactive display and/or implemented using virtual reality or artificial reality platforms. As previously discussed, as exemplified in FIG. 16, the GUI 214C may display impingement, load, and/or contact points of the installed implant in a joint (e.g., a shoulder joint or hip joint), and intraoperative algorithms 5000 may determine adjustments and/or recommendations (e.g., by updated procedure plan 7020 and/or 8020), which may also be displayed visually and/or via textual instructions.

As exemplified in FIG. 17, the GUI 214E may be configured to display patient specific data (e.g., preoperative data 1000 or intraoperative data 2000, such as biometrics 1110 and/or 2050) and steps, targets, instructions, or other parameters of the preoperatively determined procedure plan 7020. This information may be updated intraoperatively, for example, when intraoperative algorithms 5000 create the intraoperatively determined procedure plan 8020. GUI 214 may also be configured to display operating room efficiency 2010 data.

The intraoperative data 3000, figures, illustrations, animations, and/or videos displayed on the GUI 214 may be recorded and stored on the memory system 40, and the preoperative algorithms 4000, intraoperative algorithms 5000, and/or postoperative algorithms 6000 may be configured to learn or be trained on patterns and/or other relationships across a plurality of patients in combination with intraoperative outputs 8000, postoperative data 3000 (e.g., patient outcome 3010), and postoperative outputs 8000. The learned patterns and/or relationships may refine determinations made by the preoperative algorithms 4000, intraoperative algorithms 5000, and/or postoperative algorithms 6000, which may further refine displays on the GUI 214 (e.g., bone recognition and/or determinations, targets, recognition and/or display of other conditions and/or bone offsets, etc.)

Referring back to FIGS. 1 and 3, the intraoperative outputs 8000 may include a postoperative plan 8030, which, like the intraoperative plan 8020, may be newly generated based off of intraoperative information 8000 and/or may be a modification to the postoperative plan 7030 generated using the preoperative information 7000 (and/or a manually input procedure plan 7020). For example, based on range of motion 2090, biometrics 2050, actual incision length 2060 and/or implant position 2100 or type 2110, etc., the postoperative plan 8030 may be modified to include recommended office visits, pain medications and dosages, a revision surgery, an exercise plan, etc. The postoperative plan 8030 may include the same types of information and/or parameters as the preoperatively determined postoperative plan 8030 (exercise plan, discharge plan, pain medication plan, etc.).

Similarly, the bone density score 8040, fall risk or stability score 8050, an activity quality score 8060, joint stiffness score 8070, B-score 8100, and fracture risk score 8140 may indicate (and be calculated from) similar information as the preoperatively determined bone density score 7040, fall risk or stability score 7050, an activity quality score 7080, joint stiffness score 7090, B-score 7120, and fracture risk score 7140. The patient readiness score 8080 may, however, be an assessment of a readiness to end surgery and/or a readiness to discharge (rather than a readiness to have surgery), where a lower patient readiness score 8080 may indicate that more time is needed before ending surgery and/or discharging. The preoperative algorithms 4000, intraoperative algorithms 5000, and/or postoperative algorithms 6000 my calculate the patient readiness score 8080 using procedure duration 2020 and/or blood loss 2040, in addition to similar parameters as the patient readiness score 7100.

### Postoperative Data 3000

Referring to FIGS. 3-4, the postoperative data 3000 may include information on patient outcome 3010, lifestyle 3020, patient satisfaction 3030, electromyography (EMG) 3040, planned procedures 3050 (e.g., revisions), psychosocial 3060, bone imaging 3080, bone density 3090, biometrics 3100, and kinematics 3110 including range of motion 3112 and/or alignment 3114, postoperative medical history 3129, and recovery 3130. This list, however, is not exhaustive and postoperative data 3000 may include other patient specific information and/or other inputs manually input by a practitioner. Some of the postoperative data 3000 may be directly sensed, and other postoperative data 3000 may be determined (e.g., using a postoperative algorithm 6000) based on directly sensed or input information.

Patient outcome 3010 may include both immediate and long term results and/or metrics from the medical procedure (e.g., surgery). For example, patient outcome 3010 may include a success metric or an indication of whether the procedure was successful, changes in range of motion, stability, fall risk or stability, fracture risk, joint stiffness or flexibility, or other changes between preoperative data 1000, or intraoperative data 2000 and postoperative data 3000, etc. Patient satisfaction 3030 may be a patient-reported (or, alternatively or in addition thereto, a practitioner-reported) satisfaction with the procedure, both immediate and long-term. Planned procedure 3050 may include information determined in outputting the postoperative plan 8050 and/or other information on future planned procedures for the patient (e.g., a surgeon-created plan or revision based on patient outcome 3010, etc.) Medical history 3120 may include updated and/or new medical history 3120 (as compared to preoperative medical history 1030) and may include both immediate and long term information such as new utilization of orthotics, care information in a supervised environment such as a skilled nursing facility or SNF, infection information, etc. Information on recovery 3130 may include information on adherence to a postoperative plan 8030 such as actual exercises performed, medicine dosage and/or type actually taken, fitness information, planned physical therapy (PT), adherence to PT, etc. Information on recovery 3130 may also include discharge and/or length of stay information.

Lifestyle 3020, EMG 3040, psychosocial 3060, bone imaging 3080, bone density 3090, biometrics 3100, kinematics 3110, range of motion 3112, and/or alignment 3114 may include similar types of information as preoperative lifestyle 1020, EMG 1040, psychosocial 1060, bone imaging 1080, bone density 1090, biometrics 1100, kinematics 1100, range of motion 1112, and alignment 1114. For example, psychosocial 3060 may include perceived pain, stress, happiness, anxiety, etc.

### Postoperative Measurement System 300

Referring to FIGS. 2, 4, and 19, the system 20 may collect postoperative data 3000 from the postoperative measurement system 300. Like the preoperative measurement system 100, the postoperative measurement system 300 may include electronic medical records (EMR) 302, patient/user interfaces or applications 304, diagnostic imaging systems 306, mobile devices 308, a motion sensor and/or kinesthetic sensing systems 314, and electromyography or EMG systems 320. Like the intraoperative measurement system 200, the postoperative measurement system 300 may include one or more sensored implants 316, and a sensored patient bed 318. The devices implementing EMR 302, patient/user interfaces 304, diagnostic imaging systems 306, mobile devices 308, motion sensor system 314, EMG system 320, sensored implant 316, and sensored bed 318 of the postoperative measurement system 300 may each include one or more communication modules (e.g., WiFi modules, BlueTooth modules, etc.) configured to transmit postoperative data 3000 to the memory system 40, the system 20, to each other, etc.

EMR 302 may include any of the features of preoperative EMR 102 and intraoperative EMR 202, and may include updated records including postoperative observations by one or more practitioners performing the procedure plan 7020. The system 20 may use EMR 302 to collect information on postoperative medical history 3120, patient outcome 3010, lifestyle 3020, recovery 3130, planned procedures 3050, etc. Patient and/or user interfaces 304 may be similar to preoperative user interfaces 104. Patient interfaces 304 may present questionnaires, surveys, or other prompts for patients to enter psychosocial information 3060 such as perceived pain, stress level, anxiety level, feelings, and other patient reported outcome measures (PROMS). Patients may also report lifestyle information 3020 via patient interfaces 304. These patient interfaces 304 may be executed on other devices disclosed herein (e.g., using mobile devices 308 or other computers). Diagnostic imaging systems 306 may be similar to preoperative diagnostic imaging systems 106, and the system 20 may use diagnostic imaging systems 306 to collect bone imaging information 3080, including morphology and/or anthropometrics, fractures, and bone density 3090.

Mobile devices 308 may include smartwatches 310 or smartphones 312 and be the same as or have any of the features of mobile devices 108 used preoperatively. The system 20 may use mobile devices 308 to measure biometrics 3100, kinematics 3110, psychosocial information 3060, lifestyle information 3020, etc. by including sensors that measure heart rate, electrocardiogram data, breathing rate, temperature, oxygenation, sleep patterns, activity frequency and intensity, and or by providing survey prompts and/or patient interfaces 304.

The system 20 may use EMG systems 320 to collect EMG data 3040 and may be similar to preoperative EMG systems 116. Motion sensor and/or kinesthetic sensing systems 314 may be similar to preoperative motion sensor and/or kinesthetic sensing systems 114 and include motion capture (mocap) systems, external motion sensors, and wearable sensors to measure kinematics 3110 and range of motion 3112 data. Motion sensor and/or kinesthetic sensing systems 314 may include kinematics tracking systems which are the same or similar to kinematics tracking systems 120 and 130 used preoperatively. The system 20 may use other types of stimulation systems (e.g., configured for a kinematic or EMG response) to collect postoperative data 3000.

Sensored implants 316 may be the same or include any of the features of permanent sensored implants 216 used intraoperatively. As an example, one or more temporary or trial implants 216 may be used intraoperatively to collect intraoperative data 2000, and a permanent implant 216 may be installed toward the end of the preoperatively determined surgical procedure 7020 and/or intraoperatively determined surgical procedure 8020. Postoperative implants 316 may be the same devices as the permanent implants 216 installed during surgery intraoperatively. Like intraoperative sensored implants 216, the system 20 may use postoperative sensored implants 316 to collect kinematics 3110, range of motion 3112, and alignment 3114 (e.g., if an implant 316 becomes dislodged or misaligned). The system 20 may also use sensored implants 316 to detect a presence of an infection or an infection rate at or near where the sensored implant 316 is installed by, for example, using sensors that detect changes in synovial fluid, blood glucose, body temperature, and/or using electrodes that detect current information, ultrasonic sensors that detect other nearby structures, etc.

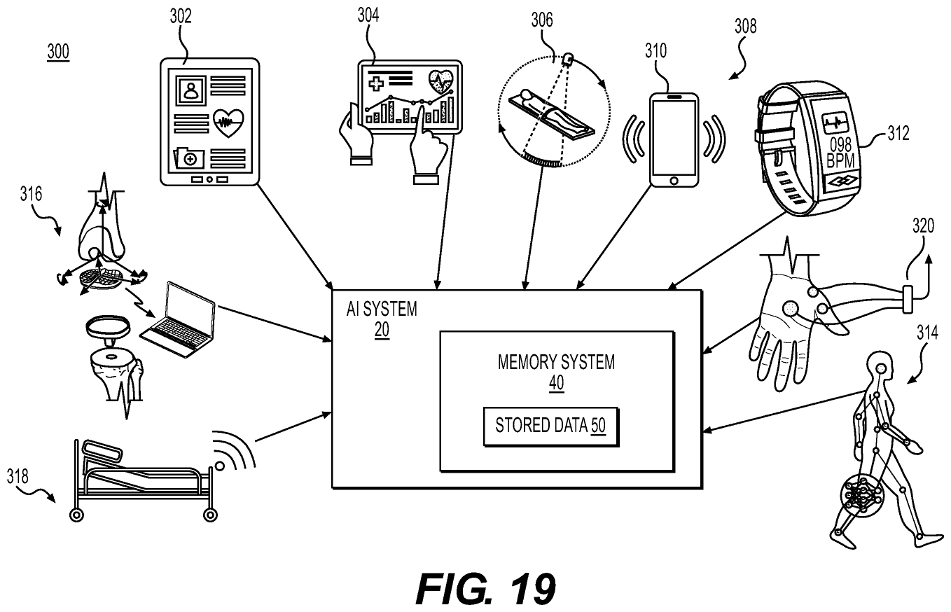

Referring to FIGS. 19-20, the postoperative sensored bed 318 (e.g., hospital bed, discharge bed, etc.) may be a moveable bed with multiple sensors to detect activity level and/or biometrics of a patient. The sensored bed 318 may include a bed frame or base 320 and a plurality of wheels 322, 323 to move the bed frame 320. The plurality of wheels 322, 323 may include at least one front wheel 322 and at least one back wheel 323. As exemplified in FIGS. 19-20, the sensored bed 318 may include a pair of front wheels 322 and a pair of back wheels 323. At least one of the front wheel 322 or the back wheel 323 may be drive. For example, the sensored bed 318 may include a motor to drive the front wheels 322 for automated movement or transport, while the back wheels 323 may be driven wheels.

The bed frame 320 may include a mattress frame or support 324 configured to receive a mattress 326 (FIG. 20). The mattress frame 324 and mattress 326 may be divided into sections corresponding to a patient's body (e.g., head area, torso area, pelvis area, thigh area, calf area, foot area, etc.), and each section may be pivotable with respect to adjacent sections for adjustment (by, for example, motors and/or actuators configured to drive links, rails, etc. coupled to or included in the frame 320 and/or mattress frame 324). As shown in FIG. 20, each section and/or the mattress frame 324 as a whole may also be raised and/or lowered using, for example, elevation adjusters 325 including actuators, pneumatic pumps, etc. Aspects disclosed herein are not limited to a design of the frame 320 and/or mattress 326. As an example, U.S. Pat. No. 10,687,999 describes a sensored bed, which is incorporated herein by reference.

At least one of the mattress frame 324 and/or mattress 326 may include a plurality of sensors 327, 329. The plurality of sensors 327, 329 may include a force sensor (e.g., load cell), optical sensor (e.g., laser sensor or infrared sensor), potentiometer, gyroscope-based sensor, accelerometer, magnetic sensor (e.g., Hall sensor or proximity sensor), a capacitive sensor, touch tape, a switch (e.g., a limit switch), etc. An arrangement of the plurality of sensors 327, 329 may be configured to measure loads, magnetic forces, capacitance, light, etc. at a plurality of positions. The arrangement of the plurality of sensors 327, 329 is not limited to the exemplary arrangement shown in FIGS. 20-21. These sensors 327, 329 may be used to measure biometrics 3100 (e.g., sleeping patterns, breathing rate) and kinematics 3110 (e.g., an amount of activity or movement, entrance/exit data, posture and/or body alignment data, etc.). For example, the mattress frame 324 may include a plurality of sensors 327, and the mattress 326 may include a plurality of sensors 329. The plurality of sensors 327, 329 may be implemented as load cells provided at a plurality of positions in the various sections of the mattress frame 324 and/or mattress 326 to determine a weight at a plurality positions, and from this data, an orientation of the patient's body may be determined, in addition to, over time, movement of the patient's body based on changes. Movement patterns may be used to determine sleeping patterns. Slight changes in movement may indicate breathing patterns.

The mattress frame 324 may also include sensors 329 configured to measure pulse or heart rate (e.g., upon contact of a patient's finger, etc.) The plurality of sensors may also be used to detect a moisture level on the skin, temperature, etc. Some of the data from the sensors may be used to determine psychosocial 3060 data (e.g., anxiety or stress data based on sleeping patterns) or to calculate related postoperative outputs 9000 described later. Alternatively or in addition thereto, sensored pillows and/or bed sheets, quilts, etc. may be used with the frame 320 and mattress 326. Data from these sensors may be combined with other wearable or attachable sensors used in hospitals to monitor patients (e.g., heart rate monitors, pulse oximeters, etc.).

### Postoperative Outputs 9000

Referring to FIGS. 1 and 3-4, the postoperative outputs 9000 may be determined via one or more postoperative algorithms 6000. The postoperative algorithms 6000 may also consider preoperative information 1000 and/or outputs 7000, intraoperative information 2000 and/or outputs 8000, and/or other previously stored data 50 of memory system 40 to determine postoperative outputs 9000. The postoperative outputs 9000 may include an updated or new postoperative plan 9030, which may include a medication plan 9032 (e.g., for pain medication, antibiotics, etc.) and/or a discharge plan 9034, a patient readiness score 9010, an updated or new bone density score 9040, an updated or new fall risk or stability score 9050, an updated or new activity quality score 9060, an updated or new joint stiffness score 9070, an updated or new psychosocial score 9080, an updated or new B-score 9090, an updated or new push-off power score 9100, and an updated or new fracture risk score 9140. This list is not exhaustive, however. The updated or new bone density score 9040, fall risk or stability score 9050, activity quality score 9060, joint stiffness score 9070, psychosocial score 9080, B-score 9090, push-off power score 9100, or fracture risk score 9140 may include any of the features of preoperatively and intraoperatively determined bone density score 7040 and/or 8040, fall risk or stability score 7050 and/or 8050, activity quality score 7080 and/or 8060, joint stiffness score 7090 and/or 8070, psychosocial score 7110, B-score 7120 and/or 8100, push-off power score 7130, and/or fracture risk score 7140 and/or 8140, respectively. The patient readiness score 9010 may indicate a readiness to be discharged (rather than a readiness for surgery) and may be based on (and updated using) postoperative data and outputs 3000, 9000, such as patient outcome 3010, lifestyle 3020, patient satisfaction 3030, electromyography 3040, psychosocial 3060, bone imaging 3080, biometrics 3100, kinematics 3110, recovery 3130, fall risk score 9050, activity quality score 9060, psychosocial score 9080, push-off power score 9100, fracture risk score 9140, etc.

As previously mentioned, postoperative algorithms 6000 may be used to output the postoperative plan 9030. This postoperative plan 9030 may be newly generated based on postoperative data 3000 and/or may be a modification to the postoperative plan 8030 generated using the intraoperative data 2000 (and/or a manually input) and/or the postoperative plan 7030 generated using the preoperative data 1000 (and/or manually input). In this context, for example, a medical practitioner may manually input an adjustment to the postoperative plan 9030 via an electronic device. The postoperative plan 9030 may be continuously adjusted and/or updated as more postoperative data 3000 is collected.

As an example, the postoperative algorithm 6000 may determine that only minor adjustments are necessary to update the postoperative plan 8030 based on postoperative data 1000 like recover 3130, kinematics 3110, biometrics 3100, patient satisfaction 3030, lifestyle 3020, etc. As another example, unexpected responses or conditions indicated by the postoperative data 1000, which may differ from expected or optimized postoperative conditions (e.g., increased or decreased perceived pain, lower or higher range of motion 3112, unexpected injury indicated in the medical history 3120, etc.), may be analyzed and considered, and the postoperative algorithm 6000 may generate a new postoperative plan 9030 (e.g., based on stored data 50 from other patients with similar unexpected conditions). Detailed determinations are described later with reference to FIGS. 22-33.

The postoperative plan 9030 may include any of the features of the prehabilitation plan 7010 or procedure plans 7020, 8020, and/or 9020, such as an exercise program configured to decrease a recovery time of the patient. The postoperative plan 9030 may include a medication plan 9032 (e.g., pain medication plan including a type, dosage, and/or tapering of pain medication) and/or a discharge plan 9034 including a length of stay in a hospital. The medication plan 9032 may be based on psychosocial information 3060, and may further be based on biometrics 3100 (e.g., heart rate variability and/or sleep patterns).

The postoperative plan 9030 may include any of the features of the preoperatively determined postoperative plan 7030, and may include a schedule of follow-up visits with a practitioner, surgeon, physical therapist, etc. Scheduled follow-up visits may be conducted remotely with markerless motion capture sensors and/or wearable sensors 114, sensored implants 216, etc. The scheduled follow up visit may be conducted via an application installed on mobile or remote devices 108. As explained later with respect to postoperative algorithms 6000, the postoperative plan 9030 may be refined, generated, and/or updated throughout the post-operative period by postoperative algorithms 6000 based on postoperative data 3000 (e.g., kinematics 1110) and/or newly determined postoperative outputs 9000 (e.g., activity quality score 9060, etc.) obtained during scheduled follow-up visits or throughout the postoperative period. Refinement may occur at predetermined intervals, upon receiving new or predetermined postoperative data 3000, and/or continuously. The surgeon may review the collected postoperative data 3000 and/or newly determined or updated postoperative outputs 9000 (which may be stored in memory system 40) without physically meeting with the patient. For example, if the patient has not yet reached predetermined goals two weeks after surgery, the postoperative algorithms 6000 may update or determine a new postoperative plan 9030, and the practitioner may be notified of the update. The postoperative plan 9030 may also include a plan for revision surgeries or future additional surgeries, though the procedure plan 7020 may be configured to reduce a likelihood of revision surgeries. Like the procedure plan 8020, the postoperative plan 9030 may be based on preoperative outputs 7000 and intraoperative outputs 8000. In addition, the postoperative plan 9030 may be based on other postoperative outputs 9000. For example, the postoperative plan 9030 may include an exercise program configured to target muscles based on the patient's postoperative lifestyle 3020 (e.g., frequency of climbing stairs) and postoperatively determined fall risk score 9050 and/or fracture risk score 9140.

The medication plan 9032 may include instructions for pain medication or other medication (e.g., antibiotics). For example, the medication plan 9032 may include a medication type, active ingredient, mechanism of action, route of administration, dosage level, dosage plan (e.g., taper plan of dosing), frequency, and/or other instructions related to taking medication. The medication plan 9032 may be based on postoperative data 3000 and postoperative outputs 9000 (and preoperative and intraoperative analogs) such as patient outcome 3010, lifestyle 3020, patient satisfaction 3030, planned procedure 3050, psychosocial 3060, bone imaging 3080, kinematics 3110, biometrics 3100, medical history 3120, recovery 3130, discharge plan 9034, patient readiness score 9010, psychosocial score 9080, etc. For example, the medication plan 9032 may be based on a patient's prior drug history (collected from EMR 102, 302, etc.), perceived pain and/or PROMS (e.g., collected using apps or user interfaces 104 and/or 304), biometrics 3100 like heart rate variability and sleep patterns, bone imaging 3080 (e.g., fractures or healing of fractures), infections or sickness (e.g., detected from changes in synovial fluid using sensored implants 216 and/or 316, detected from sensors measuring blood glucose, body temperature, sleep disturbances, heart rate variability, etc.) and other recovery 3130 data.

The medication plan 9032 may be updated continuously and/or periodically postoperatively. For example, biometrics 3100 like certain heart rate variability patterns (e.g., higher heart rate) and/or short or infrequent sleeping patterns may indicate that the patient is experiencing a higher level of pain, and the medication plan 9032 may be updated to increase a dose or determine a different (or stronger) type of pain medication. EMG data 3040 may also provide insight into pain levels. As another example, sensored implants 216 may detect information related to infections, and the medication plan 9032 may be updated to include an antibiotic or other type of medication meant to treat the infection. Infection information may be sensed by sensored implants 216 or other sensors configured to detect a change in synovial fluid and configured to detect other biometrics 3100 such as heart rate variability, blood glucose, sleep disturbances, and body temperature which may indicate an infection at the surgical site. As another example, addictive behaviors may be determined (e.g., using biometrics 3100 and EMG data 3040 in combination with patient medical history or lifestyle 1020 and/or 3020 and/or other PROMS data), and the medication plan 9032 may be created or updated to avoid and/or taper addictive pain medication, like opioids.

The discharge plan 9034 may include instructions for immediate recovery after surgery, such as a length of a hospital stay, supervision instructions, physical therapy instructions, target outputs (e.g., a fall risk threshold or target for the fall risk score 9050, a target activity quality threshold or target for the activity quality score 9060, a target patient readiness score 9010, a push-off power threshold or garget for the push-off power 9100, and/or a fracture risk threshold or target for the fracture risk score 9140), etc. The discharge plan 9034 may be based in preoperative, intraoperative, and postoperative data and outputs 1000, 2000, 3000, 7000, 8000, an/or 9000. For example, the discharge plan 9032 may be based on recovery 3130, medical history 3120, patient satisfaction 3030, patient outcome 3010, bone imaging 3080, kinematics 3110, biometrics 3100, fall risk score 9050, activity quality score 9060, bone density score 9040, patient readiness score 9010, push-off power 9100, and/or fracture risk score 9140.

For example, the discharge plan 9034 (and/or the patient readiness score 9010) may be updated using and/or based on postoperatively determined fall risk or stability score 9050 and/or postoperatively determined fracture risk score 91. The fall risk or stability score 9050 may be determined and/or updated using kinematics 3110 and biometrics 3100 using sensored hospital beds 318. The fracture risk score 9140 may be determined using fall risk score 9050 (or any inputs used to calculate the fall risk score 9050) and bone density data 3090 and/or a determined bone density score 9040. The sensored hospital beds 318 may track entry/exit data, heart rate variability, sleep patterns, etc. The fall risk score 9050 and/or fracture risk score 9140 may increase, for example, based on certain (e.g., increased) heart rate combined with exit events (e.g., sensed using contact sensors on the sensored hospital beds 318) and other kinematics data 3110 (e.g., acceleration data) from sensored implants 216. Based on an increased fall risk score 9050 and/or fracture risk score 9140, the discharge plan 9034 may be updated to increase a number of days in the hospital. Further details are described hereinafter with reference to FIGS. 22-33.

### Processes and Systems

The system 20 may be trained based on data from a plurality of patients, and may be further trained and refined for each use for a specific patient. FIG. 22 illustrates a process flow diagram of system 20 executing preoperative algorithms 4000, intraoperative algorithms 5000, and postoperative algorithms 6000 in order to optimize the outputs of system 20. For example preoperative algorithms 4000 may include a prehabilitation (“prehab”) exercise program algorithm 4010, a postop exercise program algorithm 4020, a patient expectations algorithm 4030, and/or a finite element analysis algorithm 4040. The intraoperative algorithms 5000 may include a fall risk detection algorithm 5010, a bone mineral and/or marrow density (BMD) and kinematics algorithm 5020, a multi joint kinematic assessment algorithm 5030, and/or a postop exercise program algorithm 5040. These algorithms 5010, 5020, 5030 may also be performed preoperatively, and may be included in preoperative algorithms 4000. The postoperative algorithms 6000 may include a postop exercise optimization algorithm 6010, a pain medication optimization algorithm 6020, and/or a patient discharge algorithm 6030.

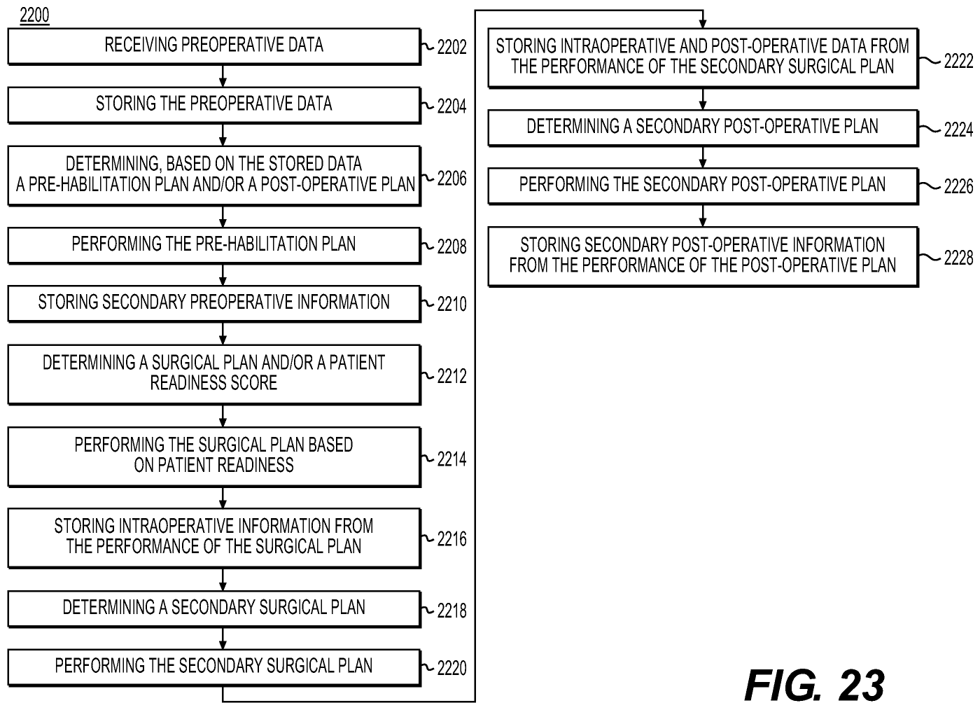

The preoperative algorithms 4000, intraoperative algorithms 5000, and postoperative algorithms 6000 may implement machine learning and/or AI to be “trained,” or may learn and refine patterns between input information 10 and output information 30, which are used to make determinations. The preoperative algorithms 4000, intraoperative algorithms 5000, and postoperative algorithms 6000 will be described in more detail in connection with the exemplary method 2200 illustrated in FIG. 23, which exemplifies preoperative, intraoperative, and postoperative steps over the course of treatment for a specific patient.

Referring to FIGS. 1-4 and 22-23, the exemplary method 2200 of FIG. 23 may be executed utilizing system 20. Method 2200 may include a step 2202 of receiving preoperative data 1000 for an instant patient. Preoperative data 1000 may be received directly from preoperative measurement system 100 and/or from previously collected and stored data 50 in the memory system 40. When preoperative data 1000 is received directly from preoperative measurement system 100, the method 2200 may include a step 2204 of storing the received preoperative data into the memory system 40. As previously described, the stored data 50 in the memory system 40 may include all types of preoperative data 1000, intraoperative data 2000, and postoperative data 3000 from a plurality of previous patients.

### Prehabilitation Plan

The method 2200 may include a step 2206 of determining a pre-habilitation plan 7010 (or “pre-hab”) and/or a postoperative plan 7030 for the instant patient. Determining the pre-habilitation plan 7010 and the postoperative plan 7030 may be based on information stored in the memory system 40, including the preoperative data 1000 for the specific patient and any other data for prior patients. The preoperative data 1000 may include data from a patient's medical history and other data known before performance of the prehabilitation plan 7010, in addition to preoperative data 1000 collected immediately before or during performance of the prehabilitation plan 7010 in step 2208, in which case, step 2206 of determining the prehabilitation plan 7010 may be repeated (e.g., periodically repeated and/or repeated based on new information stored in the memory system 50 and/or new information measured by preoperative measurement system 100).

Referring to FIG. 23, the exercise program algorithm 4010 may use bone density 1090, morphology/anthropometrics 1082, and/or other data from bone imaging 1080 (e.g., from a CT scan) to determine B-score 7102, which may be used as another input into the exercise program algorithm 4010, to determine the prehabilitation plan 7010. The exercise program algorithm 4010 may further use EMG 1040 and kinematics 1110 (and/or further determine an activity level or quality score 7080 and/or a joint stiffness score 7090, which may be used as another input) to determine the prehabilitation plan 7010. The exercise program algorithm 4010 may also determine, calculate, and/or consider a patient readiness score 7100, which may be determined before, during, or after performance of the prehabilitation plan 7010 (such as in step 2208). The prehabilitation plan 7010 may be updated or modified based on the patient readiness score 7100.

Based on these inputs, the exercise program algorithm 4010 may determine specific exercises. For example, if preoperative data 1000 (e.g., bone imaging 1080, EMG 1040, kinematics 1110, B-score 7102, etc.) indicates that a patient has a lower bone density 1090 at ligament insertion sites, low activity at a certain muscle (e.g., at the quads), and increased laxity (or decreased stiffness) at joints related to the certain muscle (e.g., knee joint), the exercise program algorithm 4010 may determine that the prehabilitation plan 7010 should include a certain number, frequency, duration, etc. of muscle strengthening exercises for the certain muscle (e.g., quad strengthening exercises like squats) before a total joint (e.g., knee) arthroplasty procedure 7020 and/or 8020 is performed intraoperatively. As another example, if a patient has lower balance and/or stability or a greater fall risk or fracture risk (as indicated by kinematics 1110, range of motion 1112, a greater fall risk score 7050, a greater fracture risk score 7140, a lower bone density 1090 and/or bone density score 7040, etc.), the exercise program algorithm 4010 may determine that the prehabilitation plan 7010 should include a certain number, frequency, duration, etc. of sit-to-stand exercises, one-legged exercises, other stability training exercises (e.g., core strengthening exercises like curl-ups or sit-ups), or regression exercises.

Aspects disclosed herein are not limited to a total knee arthroplasty procedure and may be applied to other total joint arthroplasty procedures or other procedures involving installation of a prosthesis or implant (e.g., hip, shoulder, spine, neck, wrist, ankle, foot, forearm, or other limbs). For example, with a shoulder joint replacement surgery using a shoulder prosthetic system 226, if a patient has a lower bone density 1090 at ligament insertion sites, EMG 1040 that indicates low activity at the pectoralis (or alternatively, deltoids, biceps, triceps, trapezius, etc.), and increased laxity at the shoulder joint, the exercise program algorithm 4010 may determine that the prehabilitation plan 7010 should include a certain number, frequency, duration, etc. of pectoralis (or alternatively, deltoids, biceps, triceps, etc.) exercises (like pushups, dumbbell press, bench press, barbell curls, pull-ups, hammer curls, etc.) before a shoulder joint replacement surgery 7020 and/or 8020 is performed intraoperatively. Based on low activity at muscles surrounding the pelvis and/or abdomen, the exercise program algorithm 4020 may determine that the prehabilitation plan 7010 should include a certain number, frequency, duration, etc. of abdomen strengthening exercises (e.g., sit-ups or curl-ups). Based on low activity at muscles surrounding the upper or lower back and/or the chest, the exercise program algorithm 4020 may determine that the prehabilitation plan 7010 should include a certain number, frequency, duration, etc. of back and/or chest training exercises (e.g., lat pulldowns, pull-ups, deadlifts, and dumbbell rows).

Intraoperative data 2000 and outputs 8000 and postoperative data 3000 and outputs 9000, especially patient outcome 3010 and/or whether an optimized outcome was achieved, may be stored in the memory system 40 and/or used in a subsequent determining of the prehabilitation plan 7010 by the exercise program algorithm 4010. The patient expectations algorithm 4010 may be operated postoperatively to determine whether an optimized outcome for a patient was received by comparing preoperatively and intraoperatively determined outcomes (e.g., in procedure plans 7020, 8020) to actual or postoperatively measured results and parameters (e.g., alignments, slopes, range of motion, etc.) In addition, the exercise program algorithm 4010 may be further trained and/or refined based on the intraoperative data 2000 and outputs 8000 and postoperative data 3000 and outputs 9000, which may be stored in the memory system 40. For example, based on intraoperative tourniquet time 2030 and/or blood loss 2040 for a tourniquet on the instant patient's quad (or alternatively, bicep or tricep) and/or based on patient outcome 3010, the exercise program algorithm 4010 may increase or decrease an amount of quad (or bicep or tricep) strengthening areas for a future patient because, for example, the future patient may benefit from stronger quads if the future patient's surgery will require a tourniquet on the quad (or bicep or tricep).

A relationship between tourniquet time, quad (or bicep or tricep) strength, and other patient characteristics (e.g., BMI), may be trained, learned, and refined over time by the exercise program algorithm 4010 receiving data from a plurality of patients over time. In addition, the exercise program algorithm 4010 may use thresholds (e.g., predetermined activity levels or quad strengths, predetermined times or time intervals of tourniquet time, predetermined BMI, etc.) to make determinations, and the thresholds and/or formulas for calculating thresholds may be similarly trained and refined over time. All data 1000, 2000, 3000 and outputs 7000, 8000, 9000 may be stored in the memory system 40 to facilitate training and refinement of the exercise program algorithm 4010 and other algorithms 4000, 5000, 6000 for both the instant patient and future patients.

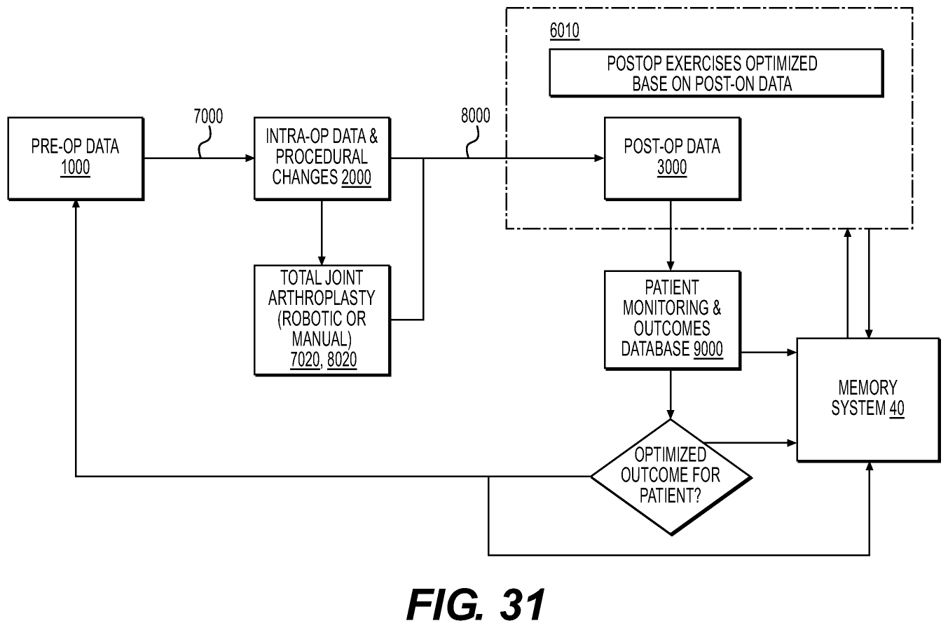

Determining the postoperative plan 7030 in step 2206 may be similar to and may include any of the features of the process described with respect to FIG. 24 using the postop exercise program plan algorithm 4020. Alternatively and/or in addition thereto, an updated or new postop plan 8030 may be determined intraoperatively, as will be described later with reference to FIG. 31.

Referring back to FIGS. 1-4 and 22-23, the method may include a step 2208 of performing the determined prehabilitation plan 7010. During performance of the prehabilitation plan 7010, more (e.g., secondary) postoperative data 1000 may be collected. The method 2200 may include a step 2210 of storing the secondary postoperative data 1000. The method 2200 may include repeating step 2206 of determining (e.g., updating) the prehabilitation plan 7010 and/or the postoperative plan 7030 based on the secondary postoperative data 1000.

### Patient Readiness and Procedure Plan

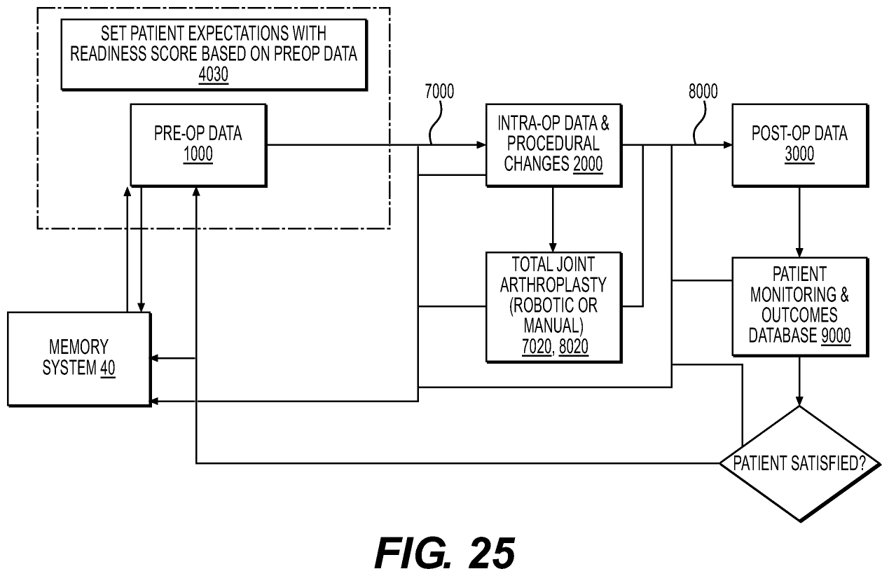

The method 2200 may include a step 2212 of determining a patient readiness score 7100 for a procedure for the instant patient. Referring to FIG. 25, this step 2212 may be performed using patient expectations algorithm 4030. The patient readiness score 7100 may indicate a patient's independence and/or readiness to undergo a procedure (e.g., surgery) or if further prehabilitation may be needed to enhance a recovery time post-operatively. The patient readiness algorithm 4030 may determine a time period (e.g., number of days) for the patient to wait for the procedure and/or determine other scheduling parameters for the procedure.

The patient expectations algorithm 4030 may consider psychosocial data 1060, kinematics 1110, range of motion 1112, and/or alignment data 1114 (e.g., data on postural sway). The patient expectations algorithm 4030 may also calculate preoperative outputs 7000 and consider the calculated preoperative outputs 7000 and/or consider preoperative outputs 7000 performed by other preoperative algorithms 4000. For example, the patient expectations algorithm 4030 may calculate a fall risk score 7050 and/or consider a fall risk score 7050 output by a fall risk algorithm (e.g., fall risk algorithm 5010 used preoperatively), calculate or consider an activity quality score 7080, calculate and/or consider a psychosocial score 7110, calculate and/or consider a joint stiffness score 7090, etc. If a patient has a lower psychosocial score 7110 (e.g., when psychosocial data 1060 indicates a high stress or anxiety level or based on certain sleeping patterns), a lower activity quality score 7080 (e.g., when a patient has less strength indicated by kinematics 1110, range of motion 1112, and/or when considering a joint stiffness score 7090), and/or a higher fall risk score 7050 (e.g., when a postural sway is higher indicated by kinematics 1110 and/or range of motion 1112), the patient expectations algorithm 4030 may calculate a lower patient readiness score 7100, indicating that a patient is not yet ready for the procedure and/or that further prehabilitation (e.g., certain exercise) is recommended before the procedure. Based on the lower patient readiness score 7100, the patient expectations algorithm 4030 may calculate a number of days until the procedure (e.g., inversely proportional to the patient readiness score 7100 or based on thresholds). Based on the lower patient readiness score 7100, the patient expectations algorithm 4030 and/or other algorithms 4000 (e.g., exercise program algorithm 4010) may make other determinations, such as certain exercises aimed to increase the patient readiness score 7100 (e.g., based on prior procedure data or activity levels at certain muscles and breathing exercises and/or other relaxation determinations such as yoga exercises based on psychosocial score 7110).

As previously described, patient expectations algorithm 4030 may make determinations which are proportional and/or inversely proportional to the patient readiness score 7100, and may be based on whether the determined patient readiness score 7100 is above or below predetermined thresholds. At least one patient readiness threshold may be based on patient readiness scores 7100 of patients having normal, typical, and/or healthy stability, psychosocial, or successful or positive patient outcome data 3010 from previous procedures with similar characteristics (e.g., demographics 1010, lifestyle 1020, medical history 1030, bone imaging 1080, biometrics 1100, type of procedure, etc.) as the instant patient.

The patient expectations algorithm 4030 may also be used postoperatively and consider post-operative patient outcome 3010 and/or satisfaction 3030 data (and/or related outputs 9000 from postoperative algorithms 6000) from previous patients, which may be stored in the memory system 40. After the procedure, postoperative data 3000 from the instant patient may be stored in the memory system 40 and/or used in a future calculation of patient readiness score 7100 for a subsequent or future patient and also to determine whether the optimized outcome for the patient was achieved and/or an extent to which an actual outcome compares to the optimized outcome based on postoperative bone density 3090, bone imaging 3080, kinematics 3110, range of motion 3112, alignment 2114, patient satisfaction 3030, psychosocial 3060 (e.g., pain), biometrics 3100, other recovery data 3130, and postoperative outputs 9000 such as bone density score 9040, fall risk score 9050, activity quality score 9060, joint stiffness score 9070, psychosocial score 9080, B-score 9090, push-off power 9100, and/or fracture risk score 9140. These values may be compared to predictive score values determined as part of the preoperative or intraoperative procedure plans 7020, 8020. As with the exercise program algorithm 4010 described with reference to FIG. 24, the patient expectations algorithm 4030 may be further trained and/or refined for the instant patient and/or future patients based on all collected and stored data 1000, 2000, 3000, and outputs 7000, 8000, 9000 may be stored in the memory system 40, including patient outcome 3010 data.

Referring back FIGS. 1-4 and 21-22, the step 2212 may also include a step of determining a procedure plan 7020 for the procedure for the instant patient. The procedure plan 7020 may include installation of an implant. The method 2200 may include a step 2214 of performing the procedure plan 7020 and a step 2216 of storing intraoperative information 2000 collected, determined, and/or measured during performance of the procedure plan 7020.

Determining the procedure plan 7020 may be performed preoperatively before the step 2214 of performing the procedure plan 7020 using a preoperative algorithm 4000 (e.g., a finite element analysis 4040 algorithm). Determining the procedure plan 7020 (and/or a step 2218 of determining a secondary procedure plan 8020) may be performed intraoperatively during performance of the procedure plan 7020 using an intraoperative algorithm 5000. Determining the procedure plan 7020 may, for example, use a preop or intraop fall risk algorithm (e.g., fall risk detection algorithm 5010), a kinematics and/or bone density algorithm (e.g., BMD & Kinematics algorithm 5020), and/or a multijoint kinematics algorithm (e.g., multi joint kinematics assessment algorithm 5030).

### Fall Risk Detection

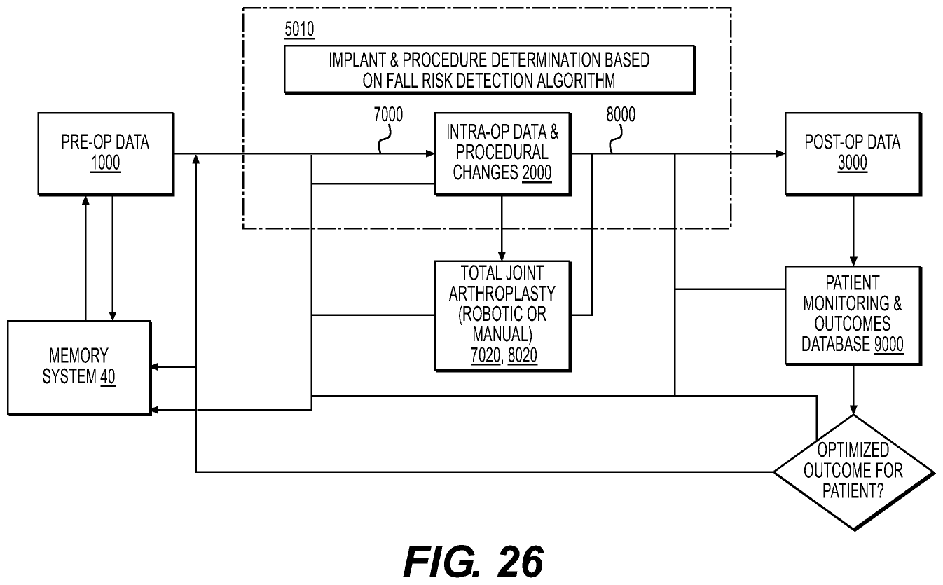

Referring to FIG. 26, the procedure plan 7020 may include a recommended design, type, and position of an implant, in addition to recommended types of procedures or tissue cuts, which may be determined based on a patient's stability or fall risk. FIGS. 22 and 26 exemplify a fall risk detection algorithm 5010 using preoperative data 1000, preoperative outputs 7000, and intraoperative data 2000 and outputs 8000, but aspects disclosed herein are not limited. The fall risk detection algorithm 5010 may be considered a preoperative algorithm 4000 (e.g., preoperatively implemented on a mobile device 108 as a fall risk app 104), in addition to or alternatively to, an intraoperative algorithm 5000 (e.g., having outputs displayed on a GUI 214) or a postoperative algorithm 6000 (for example, as part of or in conjunction with the patient discharge algorithm 6030). For example, the fall risk detection algorithm 5010 may use preoperative data 1000 and outputs 7000 to preoperatively determine at least a portion of the procedure plan 7020, and then the fall risk detection algorithm 5010 may intraoperatively update the procedure plan 7020 and/or determine a secondary procedure plan 8020 at a separate time based on intraoperative data 2000 and outputs 8000. The fall risk detection algorithm 6010 may also use preoperative data 1000 and outputs 7000 to preoperatively determine at least a portion of postoperative plan 7030, and then the fall risk detection algorithm 5010 may intraoperatively update the postoperative plan 7030 and/or determine secondary postoperative plan 8030 at a separate time based on intraoperative data 2000 and outputs 8000. The fall risk detection algorithm 6010 may further use postoperative data 3000 and outputs 9000 to postoperatively refine and/or update the postoperative plan 7030, secondary postoperative plan 8030, and/or determine postoperative plan 9030.

The fall risk detection algorithm 5010 may consider preoperative and/or intraoperative kinematics 1110 and range of motion 1112 data, such as postural sway (e.g., at the hips, knees, thighs, core, torso, neck, etc.), motion (e.g., leg, abdomen, or other core or torso motion) during daily activities (e.g., collected from wearable sensors 114 and/or sensors on a patient's mobile device 108), fall events (e.g., collected from accelerometers in various sensors such as wearable sensors 114 and/or mobile devices 108 or input by a patient or practitioner), push-off power 7130 (e.g., collected from wearable sensors 114 in a shoe and/or pressure insole sensors) and alignment data 1114 (e.g., relative spine, neck, and/or hip positions). The fall risk detection algorithm 5010 may update this data to update a fall risk score 7050 (e.g., on a fall risk app 104). The fall risk detection algorithm 5010 may also consider lifestyle data 1020 (e.g., collected via fall risk app 104 or EMR 102) and/or medical history 103 (e.g., data on prior ankle fractures or sprains, injuries to the Achilles tendon or spine, amputations and/or surgeries on toes, injuries to an ear drum canal, hearing loss information, vision information, etc.).

For example, the fall risk detection algorithm 5010 may determine a higher fall risk score 7050 based on a higher number of stairs the patient climbs or descends in an average day or over a period of time, a higher number of bending motions, a higher number of sit-to-stand movements, a higher number of one-legged movements, a higher number of lunge or squat movements, a higher number of times a patient enters or exits a vehicle or wheelchair, a higher number of reported and/or sensed fall events, a greater postural sway, a higher number and/or severity of injuries or prior procedures that affect balance (e.g., injuries to the ear drum canal, ankle, Achilles tendon, toes, torso, spine, vision, neck, ribs, or traumatic brain injury), a higher number and/or severity of diseases and/or conditions that affect balance (e.g., benign paroxysmal positional vertigo or BPPV, migraines, Meniere's disease, conditions from certain medications, etc.), a higher center of gravity of the patient (which may be considered with sex and/or gender data), speed and/or acceleration of movements determined from angular velocities, accelerations, etc., qualitative scores or other inputs based on observations by practitioners or the patient, etc. The fall risk detection algorithm 5010 may further consider determinations and/or outputs (e.g., bone density score 7040 or activity quality score 7080) from other algorithms (e.g., finite element analysis algorithm 4040, the BMD & Kinematics algorithm 5020, and/or the multi joint kinematic assessment algorithm 5030) to aggregate guidance, recommendations, instructions and/or determinations for the procedure plan 7020 and/or 8020.

The fall risk detection algorithm 5010 may include prescribed fall risk thresholds, and determine implant sizes, shapes, designs, types, a number and/or size of shims, or a fit or tightness (e.g., based on thickness, shimming, a number and/or size of shims, or desired bone slope); determine frequency, length, and position of cuts; determine frequency and slopes for bone preparation; and/or determine desired alignment (e.g., bone slopes such as tibial slope) and/or fit or tightness data based on the fall risk score 7050 and/or whether the fall risk score 7050 meets one of the prescribed fall risk thresholds. The prescribed fall risk thresholds may be trained, updated, and/or refined based on intraoperative and/or postoperative data 2000 an/or 3000 and/or based on preoperative, intraoperative, and/or postoperative outputs 7000, 8000 and/or 9000 to be used for a future or subsequent patient. At least one prescribed fall risk threshold may be based on fall risk scores 7050 of patients having normal, typical, and/or healthy stability with similar characteristics (e.g., demographics 1010, lifestyle 1020, medical history 1030, bone imaging 1080, biometrics 1100, etc.) as the instant patient.

For example, the fall risk detection algorithm 5010 may determine that a fall risk score 7050 is higher than a predetermined or prescribed fall risk threshold, which may indicate that the patient is less stable and/or has a higher risk of falling than a typical, healthy patient. When the fall risk detection algorithm 5010 detects or determines the higher fall risk score 7050, the fall risk detection algorithm 5010 may determine that the preoperatively determined procedure plan 7020 and/or intraoperatively determined procedure plan 8020 should include a stabilized or constrained implant or prostheses (e.g., a modular dual mobility or MDM implant or an anatomic dual mobility or ADM implant), should include an implant with a tighter or more constrained fit (e.g., by determining an increased thickness, diameter, or other dimensions of at least portions the implant such as length of tibial stem 233 or femoral stem 236, thickness of tibial tray 235 or humeral tray 246, thickness of measurement device 244 or insert 230, diameter of ball joint 238 or glenoid sphere 248 as shown in FIGS. 11-13, etc., an increased number of stackable shims and/or a size of an added shim (e.g., to trays 235, 246 or measurement device 244) designed to increase thickness, and/or an alignment thereof with less or shallower bone slope (e.g., tibial slope)), and/or may determine steps for practitioner and/or robot 210 to perform a soft-tissue persevering procedure and/or a more minimally invasive procedure (e.g., with less and/or shorter cuts to tissue, ligaments, etc.) The fall risk detection algorithm 5010 may also include a position and/or alignment of the implant and/or prosthesis. The fall risk detection algorithm 5010 may make determinations proportional to the fall risk score 7050, rather than and/or in addition to prescribed fall risk thresholds. For example, as the fall risk score 7050 increases, a number of cuts and/or a length of cuts determined in the procedure plan 7020 may decrease.

With respect to preoperatively determined postoperative plan 7030, intraoperatively determined postoperative plan 8030, and/or postoperatively determined postoperative plan 9030, the fall risk detection algorithm 5010 may determine, based on a higher determined fall risk and/or fall risk score 7060, 8050, 9050, the a patient should be discharged later and/or that the patient requires a home health aide. The fall risk detection algorithm 5010 may be used with and/or make similar determinations as the postoperative exercise plan algorithm 5040 and/or postoperative exercise optimization algorithm 6010 by, for example, determining certain exercises, a frequency of exercise, etc. based on fall risk and/or a determined fall risk score 7050, 8050, 9050.

The fall risk detection algorithm 5010 may further be configured to determine a fracture risk score 7140, 8140, 9140 based on a determined fall risk score 7050, 8050, 9050 and bone density data 1090, 3090, determined bone density scores 7040, 8040, 9040 and/or B-scores 7120, 8100, 9090 Alternatively or in addition thereto, the BMD & Kinematics Algorithm 5020 may be configured to determine fracture risk score 7140, 8140, 9140. The fall risk detection algorithm 5010 may make determinations based on the determined fracture risk score 7140, 8140, 9140 similar to or in conjunction with determinations based on the determined fall risk score 7050, 8050, 9050 (e.g., as the fracture risk score 7140 increases, a number of cuts and/or a length of cuts determined in the procedure plan 7020 may decrease).

As with the other algorithms 4000, 5000, 6000 described herein, the fall risk detection 5010 may be further trained and/or refined for the instant patient and/or future patients based on all data 1000, 2000, 3000 and outputs 7000, 8000, 9000 may be stored in the memory system 40, including patient outcome 3010 data.

### BMD and Kinematics

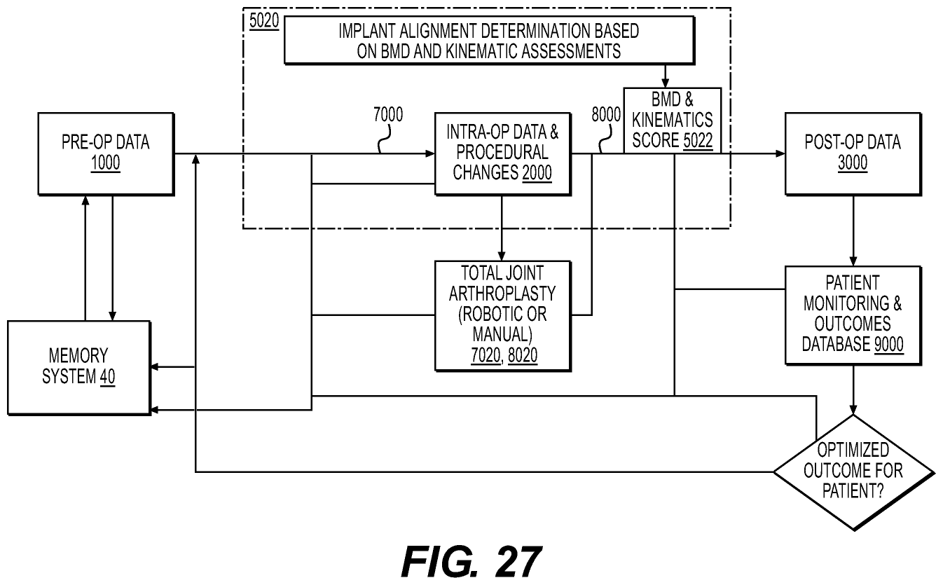

Referring to FIG. 27, the procedure plan 7020 (e.g., design, type, and position of an implant and/or certain types of procedures or tissue) may also be determined based on a patient's bone density (e.g., bone mineral density), kinematics (e.g., joint stiffness or laxity), and/or fracture risk. FIGS. 22 and 27 exemplify a BMD & Kinematics algorithm 5020 using preoperative data 1000, preoperative outputs 7000, and intraoperative data 2000 and outputs 8000, but aspects disclosed herein are not limited. The BMD & Kinematics algorithm 5020 may be considered a preoperative algorithm 4000 (e.g., preoperatively implemented on a mobile device 108 as a kinematics app 104), in addition to or alternatively to, an intraoperative algorithm 5000 (e.g., having outputs displayed on a GUI 214). For example, the BMD & Kinematics algorithm 5020 may use preoperative data 1000 and outputs 7000 to preoperatively determine at least a portion of the procedure plan 7020, and then the BMD & Kinematics algorithm 5020 may intraoperatively update the procedure plan 7020 and/or determine secondary procedure plan 8020 at a separate time based on intraoperative data 2000 and outputs 8000.

The BMD & Kinematics algorithm 5020 may consider preoperative and/or intraoperative data on bone imaging 1080, morphology/anthropometrics 1082, bone density 1090, kinematics 1110, and/or range of motion 1112 data, such as postural sway, motion during daily activities (e.g., collected from wearable sensors 114 and/or sensors on a patient's mobile device 108), push-off power 7130 (e.g., collected from wearable sensors 114 and/or pressure insole sensors) and alignment data 1114 (e.g., relative spine, neck, and/or hip positions). The BMD & Kinematics algorithm 5020 may calculate and/or consider preoperative and/or intraoperative outputs 7000 and/or 8000 such as a bone density score 7040, a B-score 7120, an activity quality score 7080, a joint stiffness score 7090, and/or a push-off power score 7130.

The BMD & Kinematics algorithm 5020 may further consider determinations and/or outputs (e.g., fall risk score 7050) from other algorithms (e.g., finite element analysis algorithm 4040, the fall risk detection algorithm 5010 and/or the multi-joint kinematic assessment algorithm 5030) to aggregate instructions and/or determinations for the procedure plan 7020 and/or 8020 and/or to modify the procedure plan 7020, 8020 based on, for example, fracture risk 7140, 8140 determined based on fall risk and bone density.

The BMD & Kinematics algorithm 5020 may determine, receive, or store prescribed bone density, B-score, range of motion, joint stiffness or laxity, activity quality, push-off power, and/or fracture risk thresholds (or predetermined values) for a bone density score 7040, B-score 7120, range of motion score, joint stiffness score 7090, activity quality score 7080, push-off power score 7130, and/or fracture risk score 7140, respectively. The BMD & Kinematics algorithm 5020 may determine other types of characteristics or scores, such as bone softness or hardness scores or an impact score based on an indentation test. Alternatively or in addition thereto, the BMD & Kinematics algorithm 5020 may determine a BMD & Kinematics score 5022, which may be based on bone density score 7040, B-score 7120, range of motion score, joint stiffness score 7090, activity quality score 7080, push-off power score 7130, fracture risk score 7140, and/or fall risk score 7050 and/or from the preoperative and intraoperative data 1000 and 2000 previously described. The BMD & Kinematics algorithm 5020 may determine, receive, or store a BMD & Kinematics score threshold, which may be compared to a determined BMD & Kinematics score 5022 for an instant patient.

The BMD & Kinematics algorithm 5020 may determine implant positions, alignment, stability, or fit/tightness steps (e.g., desired fit and/or bone slopes, thickness, diameters, a number and/or dimension of shims, etc.), frequency, length, a position of cuts, and/or determine steps for bone preparation (e.g., slopes or depths of cuts) based on determined scores (e.g., joint stiffness score 7090), a BMD & Kinematics score 5022, and/or whether determined scores or the BMD & Kinematics score 5022 meet a prescribed BMD & Kinematics threshold (among, for example, a plurality of prescribed BMD & Kinematics thresholds). For example, the BMD & Kinematics algorithm 5020 may determine that a stiffness of a joint and/or a determined joint stiffness score 7090 and/or 8070 is below a stiffness threshold (and/or predetermined stiffness value) and/or that the laxity of the joint is above a laxity threshold (and/or predetermined laxity value). The BMD & Kinematics algorithm 5020 may further determine that a bone density, a bone density score 7040, 8040, fracture risk, and/or fracture risk score 7140, 8140 is less than bone density and/or fracture risk thresholds, or predetermined bone density and/or fracture risk. Alternatively or in addition thereto, the BMD & Kinematics algorithm 5020 may further determine that a B-Score 7120 and/or 8100 is less than a B-Score threshold and/or a predetermined B-score value. Based on these determinations, the BMD & Kinematics algorithm 5020 may determine that the implant should have a fit which is tighter or more stable than a predetermined fit (e.g., by a thickness above a predetermined thickness, that the implant should be aligned with a slope below a predetermined slope, that the implant should be a certain type of implant such as a VVC, MDM, and/or ADM implant, that a shim should be added and/or a size of an added shim should be above a predetermined size, etc.)

As an alternative to thresholds, the BMD & Kinematics algorithm 5020 may make determinations which are proportional to the determined scores. For example, the BMD & Kinematics algorithm 5020 may determine a thickness of the implant and/or prosthesis which is proportional to determined laxity, inversely proportional to joint stiffness score 7090 and/or 8070, and inversely proportional to B-score 7120, 8100, bone density score 7040, 8040, and/or fracture risk score 7140, 8140. As another example, the BMD & Kinematics algorithm 5020 may determine a slope to which the implant is aligned which is inversely proportional to determined laxity, proportional to joint stiffness score 7090 and/or 8070, and proportional to B-score 7120 and/or 8100, the bone density score 7040 and/or 8040, and/or the fracture risk score 7140 and/or 8140.

The prescribed BMD & Kinematics thresholds may be trained, updated, and/or refined based on intraoperative and/or postoperative data 2000 and/or 3000 and/or based on preoperative, intraoperative, and/or postoperative outputs 7000, 8000, and/or 9000 to be used for a future or subsequent patient. At least one prescribed BMD & Kinematics threshold may be based on BMD & Kinematics scores 5022 of patients having normal, typical, and/or healthy stability with similar characteristics (e.g., demographics 1010, lifestyle 1020, medical history 1030, bone imaging 1080, biometrics 1100, etc.) as the instant patient.

As previously described, the BMD & Kinematics algorithm 5020 may determine an optimal or ideal alignment, fit, and/or position of an implant. For example, the BMD & Kinematics algorithm 5020 may determine or consider a low BMD & Kinematics score 5022 for a patient undergoing a total joint (e.g., knee) arthroplasty, which may be based on low bone density at ligament insertion points near the joint (e.g., knee), low joint stiffness and/or high joint laxity at the joint (e.g., knee), and/or a diagnosis of related conditions at the joint (e.g., polycystic kidney disease (PKD), which tends to result in high knee laxity), other laxity data (e.g., high coronal laxity), and/or higher determined fracture risk. Laxity and/or joint stiffness may be assessed from kinematics or range of motion data (e.g., 1110, 1112, 2090) using wearable sensors 114 preoperatively, and/or surgical tools 220 and/or sensored implants 216 intraoperatively. The BMD & Kinematics algorithm 5020 may determine, based on a BMD & Kinematics score 5022 less than a predetermined BMD & Kinematics threshold, that the implant (e.g. sensored implant 216) should be aligned with a smaller or shallower bone slope at the joint (e.g., tibial slope), that the implant should be designed to have a thicker shape or larger diameter (such as, with reference to FIGS. 11-13, an increased thickness, diameter, or other dimensions of at least portions the implant such as length of tibial stem 233 or femoral stem 236, thickness of tibial tray 235 or humeral tray 246, thickness of measurement device 244, diameter of ball joint 238 or glenoid sphere 248, etc., an increased number of stackable shims and/or a size of an added shim (e.g., to trays 235, 246 or measurement device 244 or insert 230) designed to increase thickness), and/or that the surgical procedure 7020 should include less cuts to allow for a tighter joint postoperatively. Alternatively or in addition to thresholds, the BMD & Kinematics algorithm 5020 may determine slopes (e.g., tibial slopes), cut frequency, cut length, etc. as a function (e.g., directly or indirectly proportional to) of the BMD & Kinematics score 5022). For example, the lower the BMD & Kinematics score 5022, the shallower the determined tibial slope and/or the thicker the implant or prosthetic in the procedure plan 7020 and/or 8020.

The patient outcome 3010 and/or resulting joint stiffness at the joint (here, knee joint) (e.g., via joint stiffness score 8070 and/or 9070) may be assessed using pressure or load sensors (e.g., in sensored implants/prosthetics 216) and/or other range of motion and kinematics data 2090, 3112, and/or 3110 collected intraoperatively and also postoperatively. Based on intraoperative joint stiffness score 8070 and/or fracture risk score 8140, the BMD & Kinematics algorithm 5020 may update the procedure plan 7020 and/or generate intraoperative procedure plan 8020. Based on postoperative joint stiffness score 9070 and/or fracture risk score 9140, the BMD & Kinematics algorithm 5020 may determine a revision procedure plan (e.g., included in postoperative plan 9030), and postoperative data 3000 and outputs 9000 may further be stored in the memory system 40 to be considered by the BMD & Kinematics algorithm 5020 for a subsequent patient.

Although an example at the knee is described, aspects disclosed herein may determine implant parameters for other joint surgeries (e.g., hip, shoulder, neck, spine, wrist, ankles, forearm, or other limbs). As with the other algorithms 4000, 5000, and 6000 described herein, the BMD & Kinematics algorithm 5020 may be further trained and/or refined for the instant patient and/or future patients based on all data 1000, 2000, and 3000 and outputs 7000, 8000, and 9000 may be stored in the memory system 40, including patient outcome 3010 data and/or whether an optimized patient outcome was achieved.

### Multi-Joint Kinematics

Referring to FIGS. 1-4, 22, and 28, the procedure plan 7020 may also be determined based on kinematics information (e.g., preoperative kinematics 1110, range of motion 1112, joint stiffness score 7090, activity quality score 7080, push-off power score 7130, and/or intraoperative kinematics/range of motion 2090, activity quality score 8060, joint stiffness score 8070, postural sway and/or fall risk score 7050 and/or 8050, fracture risk score 7140 and/or 8140, etc.). The procedure plan 7020 may include a design, type, position, alignment, fit or tightness, etc. of an implant for a first joint based on kinematics 1110, 1120, 2090, etc. for the first joint and also a second joint.

When a first joint is weaker, is in a diseased state, or is otherwise not functioning as well as it would in a healthy individual, a patient may overuse a second joint to compensate, which may cause increased wear on the second joint and/or also affect how an implant or prosthetic will move or wear over time once installed (either at the first joint or the second joint). The Multi-Joint Kinematics Assessment algorithm 5030 may use kinematics 1110, 2090 at multiple joints (e.g., hip and pelvic spine, or knee and hip), in addition to fall risk scores 7050, 8050, fracture risk scores 7140, 8140, push-off power 7130, and/or lifestyle data 1020. The Multi-Joint Kinematics Assessment algorithm 5030 may use this information to determine an alignment, position, and/or design (e.g., constrained type of implant, or an implant configured to counteract forces or motion to stabilize movement) of an implant at one of the joints (e.g., knee) to account for how multiple joints work together (or how multiple joints are expected to work together post-op) and/or compensate during certain types of motion as the patient goes about his or her daily activities.

As an example, increased pelvic spine stiffness has been found to correlate to higher hip abduction moments. The Multi-Joint Kinematics Assessment algorithm 5030 may determine an alignment, position, or design (e.g., thickness, diameter, shim arrangement, or shape) of at least a portion of the hip implant (e.g., thickness, length, shim arrangement, or shape of femoral stem 236, diameter of ball joint 238, etc. as shown in FIG. 12) based on kinematics 1110, 2090 and a joint stiffness score 7090, 8070 at the spine, in addition to the hip, to account for higher hip abduction moments. As another example, the Multi-Joint Kinematics Assessment algorithm 5030 may determine an alignment, position, or design (e.g., thickness, length, shim arrangement, or shape for tibial stem 233, tibial tray 235, femoral implant 228, or insert 230 as shown in FIG. 11) of at least a portion of a knee implant based on pelvic-spine stiffness (indicated by, for example, kinematics 1110, 2090 and a joint stiffness score 7090, 8070 at the pelvis and/or spine). In such an example, where pelvic-spine stiffness is higher, the Multi-Joint Kinematics Assessment algorithm 5030 may determine that the knee implant should be more constrained (e.g., a valgus-valgus constrained implant or VVC implant) or provide for a tighter fit (e.g., by increasing thickness).

Motion and/or stiffness in one joint has been found to affect motion and/or stiffness in a proximal joint. For example, ankle torque has been found to affect knee torque, and knee torque has been found to affect hip torque. Ankle stiffness may affect knee stiffness, and knee stiffness may affect hip stiffness. If a patient has ankle osteoarthritis (OA), the patient may pronate more or less during walking to relieve pain. A pronation angle and/or a supination angle at the ankle may impact a varus and/or valgus tilt at the knee. The Multi-Joint Kinematics Assessment algorithm 5030 may use joint stiffness score 7090 at the ankle and/or range of motion 1112 data of the patient obtained during walking to determine a target knee alignment configured to neutralize a load at the ankle to reduce or minimize OA progression, and may further determine a type, alignment, position, design (e.g., thickness, size, shim arrangement, or shape) of a knee implant to be used.

If the patient is overpronated at the ankle, then the patient may have more of a valgus tilt at the knee, which may be compensated with an implant that facilitates a tighter or more constrained movement at the knee. The Multi-Joint Kinematics Assessment algorithm 5030 may determine, based on kinematics 1110, 2090 and/or a joint stiffness score 7090, 8070, that the patient is overpronated and/or oversupinated and/or determine overpronation and/or supination angles at the ankle, and then determine the procedure plan 7020 based on overpronation and/or oversupination. For example, The Multi-Joint Kinematics Assessment algorithm 5030 may determine that the implant should have a tighter fit based on whether the ankle is overpronated and/or in proportion to the determined overpronatation angle. Based on a determination that the ankle is overpronated, the Multi-Joint Kinematics Assessment algorithm 5030 may determine that the implant should be a constrained type of implant or a stabilized implant, should have a certain thickness or a certain number of shims, a certain orientation with respect to the tibia and/or femur, etc. As another example, based on a determination that the ankle is overpronated (or oversupinated), the Multi-Joint Kinematics Assessment algorithm 5030 may determine a valgus (or varus) tilt, and determine certain cut locations, dimensions, and/or orientations in the tibia and/or femur to accommodate the implant.

In making determinations, the Multi-Joint Kinematics Assessment algorithm 5030 may use preoperative data 1000, preoperative outputs 7000, and intraoperative data 2000 and outputs 8000 to determine a multi joint kinematics score 5032 and/or separate first and second joint scores for first and second joints, but aspects disclosed herein are not limited. The Multi-Joint Kinematics Assessment algorithm 5030 may be considered a preoperative algorithm 4000 (e.g., preoperatively implemented on a mobile device 108 as a kinematics app 102), in addition to or alternatively to, an intraoperative algorithm 5000 (e.g., having outputs displayed on a GUI 214). For example, the Multi-Joint Kinematics Assessment algorithm 5030 may use preoperative data 1000 and outputs 7000 to preoperatively determine at least a portion of the procedure plan 7020, and then the Multi-Joint Kinematics Assessment algorithm 5030 may intraoperatively update the procedure plan 7020 and/or determine secondary procedure plan 8020 at a separate time based on intraoperative data 2000 and outputs 8000.

The Multi-Joint Kinematics Assessment algorithm 5030 may consider preoperative and/or intraoperative data on bone imaging 1080, morphology/anthropometrics 1082, kinematics 1110, and/or range of motion 1112, 2090 data, such as postural sway, motion during daily activities (e.g., collected from wearable sensors 114, sensors on a patient's mobile device 108, from sensored implant 216, and/or from surgical tools 220), and alignment data 1114 (e.g., relative spine, neck, and/or hip positions). The Multi-Joint Kinematics Assessment algorithm 5030 may calculate and/or consider preoperative and/or intraoperative outputs 7000, 8000 such as an activity quality score 7080, fall risk score 7050, fracture risk score 7140, and/or a joint stiffness score 7090. The Multi-Joint Kinematics Assessment algorithm 5030 may further consider determinations and/or outputs (e.g., fall risk score 7050 or fracture risk score 7140) from other algorithms (e.g., finite element analysis algorithm 4040, the fall risk detection algorithm 5010 and/or the BMD & Kinematics algorithm 5020) to aggregate instructions and/or determinations for the procedure plan 7020, 8020.

The Multi-Joint Kinematics Assessment algorithm 5030 may determine, receive, or store range of motion, joint stiffness or laxity, and/or activity quality thresholds (or predetermined values) for a range of motion score, joint stiffness score 7090, and/or activity quality score 7080, respectively. Alternatively or in addition thereto, the Multi-Joint Kinematics Assessment algorithm 5030 may determine the multi joint kinematics score 5032 (and/or first and second joint scores) based on the range of motion score, joint stiffness score 7090, and/or activity quality score 7080, and/or from the preoperative and intraoperative data 1000 and 2000 previously described. The Multi-Joint Kinematics Assessment algorithm 5030 may determine, receive, or store a multi joint kinematics score threshold, which may be compared to a determined multi joint kinematics score 5032 for an instant patient.

The Multi-Joint Kinematics Assessment algorithm 5030 may determine implant positions and/or alignment steps and implant design and/or thickness, frequency, length, and position of cuts, and/or determine steps for bone preparation (e.g., slopes of cuts) based on determinations (e.g., multi joint kinematics score 5032) and/or whether certain determined scores (e.g., the multi joint kinematics score 5032, joint stiffness score 7090, activity quality score 7080 and/or range of motion thresholds) meet prescribed corresponding thresholds (e.g., multi joint kinematics score threshold, joint stiffness threshold, activity quality threshold, or range of motion threshold). For example, with reference to FIGS. 11-13, the thickness, diameter, or other dimensions of at least portions the implant such as length of tibial stem 233 or femoral stem 236, thickness of tibial tray 235 or humeral tray 246 or femoral implant 228, thickness of measurement device 244 or insert 230, diameter of ball joint 238 or glenoid sphere 248, etc., an increased number of stackable shims and/or a size of an added shim (e.g., to trays 235, 246 or measurement device 244 or insert 230) designed to increase thickness, and/or an alignment thereof with less or shallower bone slope (e.g., tibial slope) based on the prescribed thresholds.

The prescribed thresholds may be trained, updated, and/or refined based on intraoperative data 2000 and/or postoperative data 2000 based on preoperative outputs 7000, intraoperative outputs 8000, and/or postoperative outputs 9000 to be used for a future or subsequent patient. At least one prescribed multi joint kinematics threshold may be based on multi-joint kinematics scores 5032 of patients having normal, typical, and/or healthy stability with similar characteristics (e.g., demographics 1010, lifestyle 1020, medical history 1030, bone imaging 1080, biometrics 1100, etc.) as the instant patient, which may be stored in the memory system 40 from prior procedures.

For example, with respect to a surgery involving installation of an implant at a first joint, the Multi-Joint Kinematics Assessment algorithm 5030 may determine that a stiffness of a second joint and/or joint stiffness score 7090 and/or 8070 is greater than a predetermined stiffness (or stiffness threshold) and/or a laxity of the second joint (and/or a predetermined laxity score) is less than a predetermined laxity (or laxity threshold). The Multi-Joint Kinematics Assessment algorithm 5030 may also use range of motion thresholds. For example, a range of motion (e.g., expressed as a joint angle) of the first and/or second joints may be compared to predetermined range of motions, which may be based on typical ranges of motions of a healthy or normal patient having similar characteristics as the instant patient. The Multi-Joint Kinematics Assessment algorithm 5030 may determine joint angle information for multiple segments or at multiple locations, and may determine, based on the joint angle information, kinematic constraints or relationships. The joint stiffness scores 7090, 8070 at each joint may be determined based on the range of motion comparisons, in addition to other stiffness or laxity data (e.g., gleaned from bone imaging 1080 or other data, such as a speed at which a joint may bend, etc.) The Multi-Joint Kinematics Assessment algorithm 5030 may use range of motion for the first joint in conjunction with data for the second joint or another area on the body. For example, a maximum knee flexion (e.g., of 100 degrees) combined with limited motion of the spine and/or pelvis may indicate that limited motion at the knee is due to spine/pelvis stiffness, and the Multi-Joint Kinematics Assessment algorithm 5030 may determine target alignment, treatment, or other determinations (e.g., exercises in postoperative plan 9030) relating to the spine and/or pelvis, in addition to the knee.

Based on these determinations the Multi-Joint Kinematics Assessment algorithm 5030 may determine that the installed implant should have a fit (e.g., thickness, diameter, shape, type, alignment to slope) which is tighter than a predetermined fit. For example, the Multi-Joint Kinematics Assessment algorithm 5030 may determine that a slope of a bone to which the implant is aligned should be less than a predetermined slope, a thickness of at least a portion (e.g., tray, stem, insert, or ball joint) of the implant is greater than a predetermined thickness, a number of tissue and/or bone cuts used during the procedure is less than a predetermined number, and/or the implant is a constrained type implant. Alternatively, these determinations (e.g., slope, thickness, number of tissue and/or bone cuts) may be proportional to the laxity and/or stiffness of the first and/or second joints, rather than based on thresholds.

As another example, the Multi-Joint Kinematics Assessment algorithm 5030 may determine or consider a low multi-joint kinematics score 5032 (and/or determine a score difference between first and second joint scores) for a patient undergoing total knee arthroplasty, which may be based on high joint stiffness and/or low joint laxity at the spine, and/or an indication of certain spine diseases (e.g., from EMR 102 or detection of synovial fluid). Laxity and/or joint stiffness may be assessed from kinematics or range of motion data (e.g., 1110, 1112, 2090) using wearable sensors 114 preoperatively, and/or surgical tools 220 and/or sensored implants 216 intraoperatively. The multi joint kinematics scores 5032 may determine, based on a multi joint kinematics score 5032 less than a predetermined multi joint kinematics score threshold, that the implant or prosthetic (e.g. sensored implant/prosthetic 216) should be a VVC implant, aligned with a smaller or shallower tibial slope, and/or that the surgical procedure 7020 should include less cuts to allow for a tighter knee and/or more constrained implant type postoperatively. Alternatively or in addition to thresholds, the multi joint kinematics score may determine tibial slopes, cut frequency, cut length, type or thickness of implant, shimming arrangement, etc. as a function (e.g., directly or indirectly proportional to) of the multi joint kinematics score 5032. For example, the lower the multi joint kinematics score 5032, the shallower the determined tibial slope in the procedure plan 7020 and/or 8020 and/or the thicker the type of implant 216.

The patient outcome 3010 and/or resulting joint stiffness at the knee and/or spine (e.g., via joint stiffness score 8070 and/or 9070) may be assessed using pressure or load sensors (e.g., in sensored implants/prosthetics 216) and/or other range of motion and kinematics data 2090, 3112, and/or 3110 collected intraoperatively and also postoperatively. Based on intraoperative joint stiffness score 8070, the Multi-Joint Kinematics Assessment algorithm 5030 may update the procedure plan 7020 and/or generate intraoperative procedure plan 8020. Based on postoperative joint stiffness score 9070, the Multi-Joint Kinematics Assessment algorithm 5030 may determine a revision procedure plan (e.g., included in postoperative plan 9030), and postoperative data 3000 and outputs 9000 may further be stored in the memory system 40 to be considered by the Multi-Joint Kinematics Assessment algorithm 5030 for a subsequent patient.

As with the other algorithms 4000, 5000, 6000 described herein, the Multi-Joint Kinematics Assessment algorithm 5030 may be further trained and/or refined for the instant patient and/or future patients based on all data 1000, 2000, and 3000 and outputs 7000, 8000, and 9000 may be stored in the memory system 40, including patient outcome 3010 data. Although an example of spine stiffness and higher hip abduction moments is described herein, the Multi-Joint Kinematics Assessment algorithm 5030 may recognize patterns of other joint relationships across a plurality of patients, and use data at those joints to make further determinations. For example, the Multi-Joint Kinematics Assessment algorithm 5030 might learn a certain relationship between, for example, chest strength and range of motion in the shoulder, and make determinations for a shoulder implant (e.g., thickness, tightness of fit, type of implant, shape, materials, shimming arrangement, and/or other dimensions) based on how data for the instant patient compares to the learned relationship.

### Finite Element Analysis

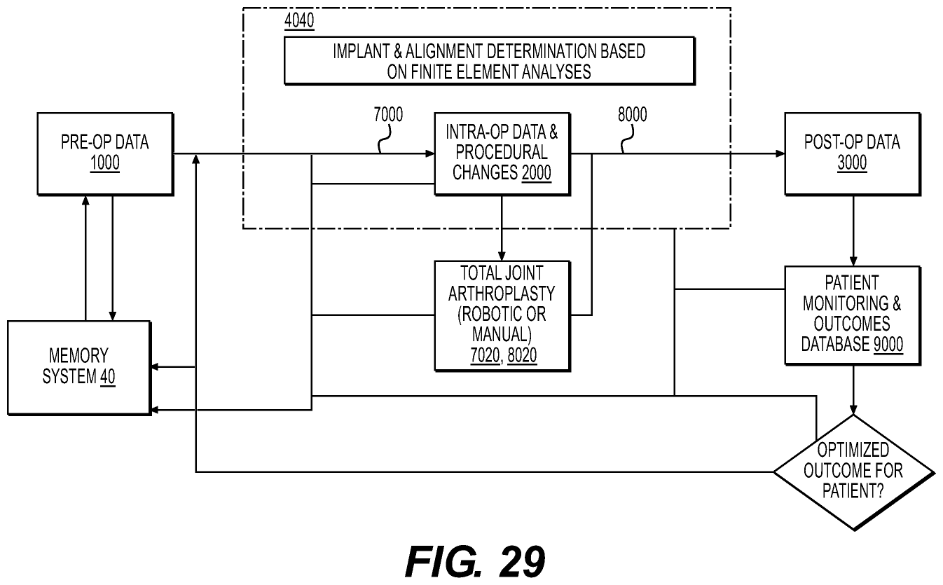

Referring to FIGS. 1-4, 22, and 29, the procedure plan 7020 (e.g., design, type, and position of an implant and/or certain types of procedures or tissue) may also be determined based on a finite element analysis. The Finite Element Analysis algorithm 4040 may use preoperative data 1000, preoperative outputs 7000, and intraoperative data 2000 and outputs 8000, but aspects disclosed herein are not limited. The Finite Element Analysis algorithm 4040 may be considered a preoperative algorithm 4000 (as illustrated in FIG. 22), in addition to or alternatively to, an intraoperative algorithm 5000 (as illustrated in FIG. 29). For example, the Finite Element Analysis algorithm 4040 may use preoperative data 1000 and outputs 7000 to preoperatively determine at least a portion of the procedure plan 7020, and then the Finite Element Analysis algorithm 4040 may intraoperatively update the procedure plan 7020 and/or determine secondary procedure plan 8020 at a separate time based on intraoperative data 2000 and outputs 8000.

The Finite Element Analysis algorithm 4040 may use finite element analysis to assist other preoperative and intraoperative algorithms 4000 and 5000 in making determinations such as the procedure plan 7020, 8020. The Finite Element Analysis algorithm 4040 may also be configured to assist postoperative algorithms 6000 in making determinations. The Finite Element Analysis algorithm 4040 may be configured to predict preoperative outputs 7000, intraoperative outputs 8000, and/or postoperative outputs 9000, and may further be configured to make determinations based on the predicted outputs 7000, 8000, 9000.

The Finite Element Analysis algorithm 4040 may consider preoperative and/or intraoperative data on bone imaging 1080, morphology/anthropometrics 1082, bone density 1090, kinematics 1110, and/or range of motion 1112 data, such as postural sway, motion during daily activities (e.g., collected from wearable sensors 114 and/or sensors on a patient's mobile device 108), and alignment data 1114 (e.g., relative spine, neck, and/or hip positions). The Finite Element Analysis algorithm 4040 may consider preoperative and/or intraoperative outputs 7000 and/or 8000 such as a bone density score 7040, a B-score 7120, an activity quality score 7080, a joint stiffness score 7090, and/or a push-off power score 7130. Finite Element Analysis algorithm 4040 may further consider determinations and/or outputs (e.g., fall risk score 7050 or fracture risk score 7140) from other algorithms (e.g., BMD & Kinematics algorithm 5020, the fall risk detection algorithm 5010, and/or the multi joint kinematic assessment algorithm 5030) to aggregate instructions and/or determinations for the procedure plan 7020 and/or 8020.

The Finite Element Analysis algorithm 4040 may, for example, use bone imaging 1080 data (e.g., from a CT scan) and preoperative and intraoperative kinematics 1110 and/or 2090 to generate a patient specific kinematic finite element model (FEM). The generated FEM may be displayed on GUI 214. The FEM may be an interactive or dynamic model showing a virtual performance of the determined procedure plan 7020 (e.g., a total joint arthroplasty). Based on outcomes or results from the model, the Finite Element Analysis algorithm 4040 may adjust the procedure plan 7020 and/or determine a new procedure plan 8020 to create certain or prescribed outcomes. For example, the Finite Element Analysis algorithm 4040 may consider native femoral rollback from preoperative kinematics data 1110 and/or intraoperative kinematics data 2090 (e.g., collected from wearable sensors 114, sensored implants 216, and/or surgical tools 220), and determine a position or alignment of an implant or prosthetic at the knee. The determined position may be configured to reduce or eliminate paradoxical motion. The determined position may differ from an initial or baseline position up to a greatest or maximum deviation allowed by an on-label use of the implant. The Finite Element Analysis algorithm 4040 may confirm that the determined position corresponds to a determine alignment that provides enhanced, optimized, or target kinematics values (e.g., based on surgeon recommendations, an original or natural knee motion, and/or other values determined by the system 20) for a patient (e.g., maximize flexion for a patient who kneels a lot).

The Finite Element Analysis algorithm 4040 may determine, receive, or store prescribed thresholds for a range of motion score, joint stiffness score 7090, activity quality score 7080, or other determinations (e.g., a collective FEM score). The Finite Element Analysis algorithm 4040 may compare determined scores for an instant patient to prescribed thresholds stored, for example, in the memory system 40.

For example, the Finite Element Analysis algorithm 4040 may determine that a joint stiffness score 7090 and/or a stability score 7050 are below prescribed corresponding thresholds (and/or that a fracture risk score 7140 or a fall risk score 7050 are above prescribed corresponding thresholds), and determine a placement and/or design (e.g., increased thickness or a shimming arrangement) of the implant in the procedure plan 7020 and/or bone preparation instructions (e.g., shallower tibial slope) configured to enhance stability and allows for a tighter installation of the implant. The prescribed thresholds may be trained, updated, and/or refined based on intraoperative and/or postoperative data 2000 an/or 3000 and/or based on preoperative, intraoperative, and/or postoperative outputs 7000, 8000 and/or 9000 to be used for a future or subsequent patient. At least one prescribed threshold may be based on scores or determinations of patients having normal, typical, and/or healthy stability with similar characteristics (e.g., demographics 1010, lifestyle 1020, medical history 1030, bone imaging 1080, biometrics 1100, etc.) as the instant patient.

The patient outcome 3010 and/or resulting joint stiffness at the knee (e.g., via joint stiffness score 8070 and/or 9070) may be assessed using pressure or load sensors (e.g., in sensored implants/prosthetics 216) and/or other range of motion and kinematics data 2090, 3112, and/or 3110 collected intraoperatively and also postoperatively. Based on intraoperative joint stiffness score 8070, the Finite Element Analysis algorithm 4040 may update the procedure plan 7020 and/or generate intraoperative procedure plan 8020. Based on postoperative joint stiffness score 9070, the Finite Element Analysis algorithm 4040 may determine a revision procedure plan (e.g., included in postoperative plan 9030), and postoperative data 3000 and outputs 9000 may further be stored in the memory system 40 to be considered by the Finite Element Analysis algorithm 4040 for a subsequent patient.

Referring back to FIGS. 1-4 and 22-23, the method 2200 may include a step 2214 of performing the procedure plan 7020. A timing of beginning the performance of the procedure plan 7020 may be based on the determined patient readiness score 7100. One or more surgeons and/or a surgical robot system 208 may perform the procedure plan 7020 by executing instructions in the procedure plan 7020.

During performing the procedure plan 7020, intraoperative data 2000 may be collected (e.g., via sensored implants 216, surgical tools 220, surgical robotic systems 208, and user interfaces 204) and analyzed, such as with the preoperative and/or intraoperative algorithms 4000 and/or 5000 previously described. Intraoperative data 2000 and/or outputs 8000 may be visualized on GUI 214. The method 2200 may include a step 2216 of storing intraoperative data 2000 and/or outputs 8000 from the performance of the procedure plan 7020.

As previously described in connection with intraoperative algorithms 5000, the method 2200 may include a step 2218 of determining a secondary procedure plan 8020. The secondary procedure plan 8020 may be a new plan and/or an updated or revised plan based on the preoperatively determined procedure plan 7020. Once a secondary procedure plan 8020 has been updated, the method 2200 may include a step 2220 of performing the secondary procedure plan 8020. Like step 2216 of performing the preoperative procedure plan 7020, the step 2220 of performing the secondary procedure plan 8020 may include collecting intraoperative data 2000 (e.g., via sensored implants 216, surgical tools 220, and surgical robot system 208) and analyzing the collected data, such as with the preoperative and/or intraoperative algorithms 4000 and/or 5000 previously described. Intraoperative data 2000 and/or outputs 8000 may be visualized on GUI 214. The intraoperative data 2000 and/or outputs 8000 from the performance of the secondary procedure plan 8020 may be stored in the memory system 40. Such intraoperative data 2000 collected may alternatively be referred to as secondary intraoperative data 2000.

The method 2200 is not limited to one secondary procedure plan 8020. For example, a second secondary procedure plan 8020 may be generated. The second secondary procedure plan 8020 may be an update or modification of the preoperatively determined procedure plan 7020, an update or modification of the secondary preoperative plan 8020, and/or newly generated based on newly collected intraoperative data 2000. The method 2200 may continuously refine and/or generate secondary procedure plan 8020 based on continuously collected intraoperative data 2000. The secondary procedure plan 8020 may be updated based on annotations or input from a surgeon.

The method 2200 may include a step 2222 storing intraoperative data 2000 and post-operative data 3000 from the performance of the secondary procedure plan 8020 (or, if no secondary procedure plan 8020 was generated, the procedure plan 7020). As previously described, postoperative data 3000 may include immediate postoperative data 3000 and also long term postoperative data 3000. Once surgery is complete, an entire course of action or steps actually performed (i.e., an actual procedure plan) may be stored in the memory system 40.

The method may include a step 2224 of determining a postoperative plan 8030, 9030. Here, the postoperative plan 8030, 9030 may be a secondary postoperative plan 8030, 9030 in comparison to preoperatively determined postoperative plan 7030. This postoperative plan 8030 and/or 9030 may be determined intraoperatively and/or based on immediate postoperative results, and another postoperative plan 9030 may be generated based on longer term postoperative results.

### Intraoperatively Determined (or Immediately Postoperatively Determined) Postoperative Exercise Plan

Referring to FIGS. 1-4, 22, and 31, the postop exercise program algorithm 5040 may use preoperative data 1000 and outputs 7000 (e.g., bone density 1090, morphology/anthropometrics 1082, and/or other data from bone imaging 1080, B-score 7102, Morphology score 7060, fall risk and/or stability score 7050, activity quality score 7080, joint stiffness score 7090, fracture risk score 7140, push-off power 7130) and also intraoperative data 2000 and outputs 8000 (e.g., Kinematics/range of motion 2090, implant position 2100, implant type 2110, soft tissue integrity 2070, bone density score 8040, fall risk score 8050, activity quality score 8060, joint stiffness score 8070, B-score 8100, fracture risk score 8140) to determine an exercise or physical therapy program as part of the postoperative plan 8030. The postop exercise program algorithm 5040 may also use immediate postoperative data 3000 and outputs 9000, such as patient outcome 3010).

For example, if a patient has a lower bone density 1090 at ligament insertion sites (which may be indicated from bone imaging data 1080 such as a CT scan), EMG data 1040 that indicates low activity at the quads, increased laxity at related joints (e.g., knee joint) indicated from intraoperative kinematics and/or range of motion data 2090, polycystic kidney disease (PKD) diagnosed intraoperatively and/or preoperatively, and/or intraoperative pressure data 2080 suggesting well-balanced movement from flexion to extension, the postop exercise program algorithm 5040 may determine that the postoperative exercise plan 8030 should include a certain number, frequency, duration, etc. of quad strengthening exercises like squats or sit-to-stand exercises. As another example, if a patient has lower balance and/or stability or a greater fall risk (as indicated by kinematics 1110, range of motion 1112, alignment 1114, push-off power 7130, a greater fall risk score 7050, etc.) or fracture risk (as indicated by kinematics 1110, range of motion 1112, push-off power 7130, alignment 1114, bone density 1090, a greater fracture risk score 7140, etc.), the postop exercise program algorithm 5040 may determine that the postoperative plan 8030 should include a certain number, frequency, duration, etc. of sit-to-stand exercises, one-legged exercises, other stability training exercises, or regression or modified exercises. Aspects disclosed herein are not limited to assessing activity at and prescribing exercises related to the knee joint. For example, the postop exercise program algorithm 5040 may determine that the postoperative exercise plan 8030 should include a certain number, frequency, duration, etc. of strengthening exercises for biceps, triceps, hamstrings, pectoralis, deltoids, trapezius, abdomen, core, glutes, etc.), low activity, increased laxity, and/or other conditions or assessments relating to those muscles and/or areas.

Long term postoperative data 3000 and outputs 9000, such as patient outcome 3010 and updated fall risk score 9050, joint stiffness score 9070, fracture risk 9140, kinematics 3110, etc. may be stored in the memory system 40 and/or used in a subsequent determining of the postoperative plan 8030 and/or 9030 by the postop exercise program algorithm 5040. In addition, the postop exercise program algorithm 5040 may be further refined and/or trained based on the postoperative data 3000 and outputs 9000, as will further be described with reference to FIG. 31.

Referring back to FIGS. 1-4 and 22, the method 2200 may include a step 2226 of performing the postoperative plan 8030 (which may be secondary if a postoperative plan 7030 was preoperatively determined). Performing the postoperative plan 8030 may include performing the exercise program determined by the postop exercise program algorithm 5040 and or other aspects of the postoperative plan 8030, such as the medication plan 9032 and/or the discharge plan 9034. The method 2200 may include a step 2228 of storing further or secondary post-operative data 3000 from the performance of the post-operative plan 8030 into the memory system 40. The secondary post-operative data 3000 may include patient outcome 3010 from the performance of the post-operative plan 8030.

### Optimization of Postoperative Exercise Plan

Referring to FIGS. 1-4, 22 and 31, the postop exercise optimization algorithm 6010 may determine an optimized postoperative plan 9030, which may be modification of and/or based on the preoperatively, intraoperatively, or immediately postoperatively determined postoperative plans 7030 and/or 8030. Like the postop exercise program algorithm 5040, the postop exercise optimization algorithm 6010 may use preoperative data 1000 and outputs 7000 (e.g., bone density 1090, morphology/anthropometrics 1082, and/or other data from bone imaging 1080, B-score 7102, Morphology score 7060, fall risk and/or stability score 7050, activity quality score 7080, joint stiffness score 7090, fracture risk score 7140, push-off power 7130) and also intraoperative data 2000 and outputs 8000 (e.g., Kinematics/range of motion 2090, implant position 2100, implant type 2110, soft tissue integrity 2070, bone density score 8040, fall risk score 8050, activity quality score 8060, joint stiffness score 8070, B-score 8100, fracture risk score 8140) to determine an exercise or physical therapy program as part of the postoperative plan 8030. The postop exercise optimization algorithm 6010 may also use immediate postoperative data 3000 and outputs 9000, such as patient outcome 3010), and long term postoperative data 3000 and outputs 9000, such as kinematics 31110 data from performance of the postoperative plan 8030, lifestyle data 3020, recovery data 3130, psychosocial data 3060, updated joint stiffness score 9070, psychosocial score 9080, fracture risk score 9140, push-off power 9100, etc.

The postop exercise optimization algorithm 6010 may make determinations that are proportional to or a function of postoperative data 3000 and/or postoperative outputs 9000. For example, based on an increase or decrease in joint stiffness score 9070 throughout a physical therapy plan, the postop exercise optimization algorithm 6010 may determine that the optimized postoperative plan 9030 should include more or less sit-to-stand or quad strengthening exercises (as compared to the preoperatively, intraoperatively, or immediately postoperatively determined postoperative plans 7030 and/or 8030).

As another example, the postop exercise optimization algorithm 6010 may use thresholds and/or scores. For example, the postop exercise optimization algorithm 6010 determine, from lifestyle data 3020, a lifestyle score, quad score, or step score relating to a number of stairs the patient climbs or descends typically in a day. Alternatively or in addition thereto, the lifestyle score may be based on a number of times the patient bends down to pick things up and/or enter or exit a vehicle or wheelchair, etc. This data may be collected using wearable sensors 114, based on information from sensored implants 216 installed during surgery or other procedures, and/or based on data from sensors (e.g., gyroscopes, accelerometers, or global positioning systems (GPSs)) in mobile devices 108. The postop exercise optimization algorithm 6010 may compare the lifestyle score to a prescribed lifestyle score threshold, and if the lifestyle score is greater than the prescribed lifestyle score threshold, the postop exercise optimization algorithm 6010 may determine that the optimized postoperative plan 9030 should include more sit-to-stand or quad strengthening exercises (e.g., squats).

The actual postoperative plan performed by the patient overtime, along with patient outcome data 3010, lifestyle data 3020, and other postoperative data 3000 and postoperative outputs 9000 may be stored in the memory system 40 and/or used in a future calculation of a postoperative plan 9030 for a subsequent or future patient. The postop exercise optimization algorithm 6010 may be further trained and/or refined (e.g., adjusted functions determining a number of exercises based on determined scores, etc.) based on the information stored in the memory system 40 across multiple patients.

### Pain Medication Optimization

Referring to FIGS. 1-4, 22, and 32, as previously described, the postoperative plan 7030, 8030, 9030 may include a medication plan 9032, which may include a plan (e.g., schedule, dosage, administration route, etc.) for pain medication. The medication plan 9032 may be determined preoperatively, updated and/or newly determined intraoperatively, updated and/or newly determined immediately after surgery postoperatively, and updated, optimized, and/or newly determined postoperatively after long term postoperative data collection. For convenience of description, a situation where the medication plan 9032 is determined immediately postoperatively (either based on a previous determination or generated from raw data) and then is optimized based on data collected during performance of the postoperative plan 9030 will be described.

The pain med optimization algorithm 6020 may consider preoperative data 1000 and outputs 3000, intraoperative data 2000 and outputs 7000, and postoperative data 3000 and outputs 9000. For example, the pain medication optimization algorithm 6020 may consider tourniquet time 2030, blood loss 2040, incision length 2060, implant design or position 2100, etc., along with patient demographics 1010 (e.g., height, weight, gender), biometrics 1100 and/or 2050, and lifestyle 1020 (e.g., previous drug use) to determine a type of pain medication, administration route, dosage, and/or frequency for the patient immediately after a procedure such as surgery. The pain medication optimization algorithm 6020 may determine a higher dosage and/or a stronger type of medication based on a higher blood loss 2040, tourniquet time 2030, and incision length 2060, higher prior drug use, and/or a larger implant or potential for notching. During performance of the pain medication plan 9032, the pain medication optimization algorithm 6020 may adjust the pain medication plan 9032 (e.g., increasing or decreasing dosage, stopping medication, creating a taper plan, etc.) based on postoperative psychosocial data or scores 1060 and/or 7110 (e.g., stress level), EMG data 1040 indicating stress, biometrics 1100 indicating stress and/or unusual sleep patterns, etc.

The pain medication optimization algorithm 6020 may make determinations that are proportional to or a function of postoperative data 3000 and/or outputs 9000. For example, based on an increase or decrease in weight, tolerance to drug use, heart rate, heart rate variability, perceived pain, and/or stress or psychosocial score, the pain med optimization algorithm 6020 may determine that a dosage and/or frequency of the pain medication should be increased or decreased as compared to the pain medication plan 9032 determined immediately postoperatively.

As another example, the pain medication optimization algorithm 6020 may use thresholds and/or scores. For example, the pain medication optimization algorithm 6020 may determine the psychosocial score 7110, the EMG score 7070, a perceived pain score, a stress score, heart rate, heart rate variability, and/or a composite pain medication score and compare determined scores to corresponding thresholds. Alternatively or in addition thereto, the pain medication optimization algorithm 6020 may compare patient demographics 1010 (and/or postoperative demographics indicating, for example, a change in weight) such as weight, gender, and lifestyle (e.g., prior drug use) and compare that data to prescribed corresponding thresholds. The pain med optimization algorithm 6020 may increase (e.g., in steps), decrease (e.g., in steps), and/or determine specific amounts, frequency, dosages, administration routes, drug types (e.g., active ingredients, extended release), etc. based on whether the prescribed corresponding thresholds are met.

The actual pain medication plan performed by the patient over time, along with patient outcome data 3010, lifestyle data 3020, psychosocial data 3060, biometrics 3100, and other postoperative data and outputs 3000 and 9000 may be stored in the memory system 40 and/or used in a future calculation of a medication plan 9032 for a subsequent or future patient. The pain med optimization algorithm 6020 may be further trained and/or refined (e.g., adjusted functions determining a number of exercises based on determined scores, etc.) based on the information stored in the memory system 40.

### Patient Discharge/Length of Stay Optimization

Referring to FIGS. 1-4, 22, and 33, as previously described, the postoperative plan 7030 and/or 8030 and/or 9030 may include a discharge plan 9034, which may include a plan and/or duration for staying in a hospital after a procedure such as surgery. The discharge plan 9034 may be determined preoperatively, updated and/or newly determined intraoperatively, updated and/or newly determined immediately after surgery postoperatively, and updated, optimized, and/or newly determined postoperatively after long term postoperative data collection. For convenience of description, a situation where the discharge plan 9034 is determined immediately postoperatively (either based on a previous determination or generated from raw data) and then is optimized based on data collected during performance of the postoperative plan 9030 will be described.

The discharge plan optimization algorithm 6030 may consider preoperative data 1000 and preoperative outputs 3000, intraoperative data 2000 and intraoperative outputs 7000, and postoperative data 3000 and postoperative outputs 9000. For example, the discharge plan optimization algorithm 6030 may consider tourniquet time 2030, blood loss 2040, incision length 2060, implant position 2100, etc., along with patient demographics 1010 (e.g., height, weight, gender), biometrics 1100 and/or 2050, lifestyle 1020, fall risk or stability 7050, 8050, 9050, push-off power 7130, 9100, fracture risk 7140, 8140, 9140, etc. to determine when a patient can be discharged from the hospital after surgery. During performance of the discharge plan 9034, the discharge plan optimization algorithm 6030 may adjust the discharge plan 9034 (increasing or decreasing length of stay, number of meals, a level of supervision, a time to remain in a hospital bed versus time outside of a hospital bed, etc.) based on postoperative psychosocial data or scores 1060 and/or 7110 (e.g., stress level), EMG data 1040 indicating stress, biometrics 1100 (e.g., heart rate variability), kinematics 3110, recovery 3130, range of motion 3112, bone imaging 3080, bone density 3090, bone density score 9040, B-score 9090, fall risk score 9050, activity quality score 9060, joint stiffness score 9070, psychosocial score 9080, push-off power 9100, and/or fracture risk score 9140.

The discharge plan optimization algorithm 6030 may make determinations that are proportional to or a function of postoperative data 3000 and/or postoperative outputs 9000. For example, based on an increased fall risk score 9050, decreased activity quality score 9060, decreased push-off power 9100, increased fracture risk score 9140, increased psychosocial score 9080, and/or increased heart rate variability or stress from biometrics 1100, the discharge plan optimization algorithm 6030 may increase a number of days, hours, weeks, etc. included in the discharge plan 9034. Alternatively or in addition thereto, the discharge plan optimization algorithm 6030 may determine the patient readiness score 9010, and may determine a length of stay in the hospital that is proportional and/or inversely proportional to the determined patient readiness score 9010. The patient readiness score 9010, for example, may indicate a readiness to leave such that a lower patient readiness score 9010 indicates that the discharge plan 9034 should include a longer stay. The patient readiness score 9010 may be based on determined fall risk score 9050, fracture risk score 9140, push-off power 9100, activity quality score 9060, psychosocial score 9080, increased heart rate variability or stress from biometrics 1100, and/or from other postoperative data 3000 and/or outputs 9000. A presence of infection or an infection level sensed from sensored implants and/216 may also indicate that the discharge plan 9034 should include a longer stay and/or recommend treatment.

As another example, the discharge plan optimization algorithm 6030 may use thresholds and/or scores. For example, the discharge plan optimization algorithm 6030 may determine the patient readiness score 9010, a composite discharge score, and/or an individual fall risk score 9050, fracture risk score 9140, push-off power 9100, psychosocial score 9080, activity quality score 9060, etc., and compare determined scores to corresponding thresholds. The discharge plan optimization algorithm 6030 may increase (e.g., in steps), decrease (e.g., in steps), and/or determine specific durations (e.g., number of days, minutes, hours) for the length of stay in the discharge plan 9034 (in addition to a level of supervision and/or a recommended time of supervision) based on whether the prescribed corresponding thresholds are met. The discharge plan 9034 may be continuously or periodically updated based on measurements and determinations throughout performance of the discharge plan 9034.

The actual discharge plan performed by the patient over time, along with patient outcome data 3010, lifestyle data 3020, psychosocial data 3060, biometrics 3100, fall instances, determined fall risk score 9050, determined fracture risk score 9140, activity quality score 9060, psychosocial score 9080, patient readiness score 9010, push-off power 9100, and other postoperative data and outputs 3000 and 9000 may be stored in the memory system 40 and/or used in a future calculation of a discharge plan 9034 for a subsequent or future patient. The discharge plan optimization algorithm 6030 may be further trained and/or refined (e.g., adjusted functions determining a number of exercises based on determined scores, etc.) based on the information stored in the memory system 40.

The algorithms 4000, 5000, and 6000 described herein may be further trained and/or refined over time for the instant patient and/or future patients based on all data 1000, 2000, and 3000 and outputs 7000, 8000, and 9000 may be stored in the memory system 40, including patient outcome 3010 data. For example, the algorithms 4000, 5000, and 6000 may learn and/or determine relationships across various data and parameters and make determinations (e.g., for an implant design or tightness, for exercises to include in pre or postoperative exercise plans, for medication plans, length of stay, etc.) based on those new learned, trained, and/or determined relationships.

For an instant patient, the system 20 may use multiple preoperative algorithms 4000, intraoperative algorithms 5000, and postoperative algorithms 6000 prior to, during, and after a medical procedure to continuously monitor and track the patient and update or refine treatment. For example, referring to FIGS. 1-4 and 22-34, the memory system 40 may have stored preoperative information 1000 (e.g., demographics 1010 and medical history 1030) from a patient from EMR 102. The patient and/or a practitioner may further enter more preoperative information 1000 into user interfaces 104 (e.g., lifestyle 1020 or information on a planned procedure 1050) gleaned from observations, intake forms, etc. The system 20 may also track some information (e.g., lifestyle 1020, kinematics 1110) using mobile devices 108. The patient may undergo imaging procedures using diagnostic imaging systems 106. The system 20 may collect, from the diagnostic imaging systems 106, bone imaging 1080 information, morphology/anthropometrics 1082, bone density 1090, alignment 1114, etc. All of this information may become stored data 50 in the memory system 40.

In the preoperative context, the system 20 may use the prehabilitation exercise algorithm 4010 to determine a prehabilitation plan 7010 based on the stored data 50, which may include the preoperative data 1000 so far collected and/or stored in the memory system 40. The system 20 may, in conjunction with operating the prehabilitation exercise algorithm 4010, determine a fall risk score 7050, bone density score 7050, morphology score 7060, EMG score 7070, activity quality score 7080, joint stiffness score 7090, patient readiness score 7100, psychosocial score 7110, B-score 7120, fracture risk score 7140, and/or push-off power 7130 to determine the prehabilitation plan 7010. Alternatively or in addition thereto, the system 20 may operate intraoperative algorithms 5000 alongside the prehabilitation exercise algorithm 4010 to make these determinations and transmit these determinations to the prehabilitation exercise algorithm 4010. For example, the system 20 may execute prehabilitation exercise algorithm 4010 alongside fall risk detection algorithm 5010 to determine the fall risk score 7050 based on the stored data 50. The system 20 may also execute BMD & Kinematics algorithm 5020 and/or the multi joint kinematic assessment algorithm 5030 to determine bone density score 7050, morphology score 7060, activity quality score 7080, joint stiffness score 7090, B-score 7120, push-off power 7130, and/or fracture risk score 7130.

The system 20 may also use the finite element analysis algorithm 4040 and/or one or more of the intraoperative algorithms 5000 to determine a medical procedure or treatment plan (e.g., procedure plan 7020). Alternatively, the system 20 may determine the medical procedure later on in the preoperative period. Similarly, the system 20 may use postoperative exercise plan algorithm 4020 to determine a postoperative plan 9030 (e.g., postoperative exercise plan), earlier at initial intake and/or later on in the preoperative period. The system 20 may use patient expectations algorithm 4030 to determine an initial patient readiness and/or procedure scheduling (e.g., number of days to surgery) or to determine scores used by the prehabilitation plan algorithm 4010 (e.g., patient readiness score 7100 and/or psychosocial score 7110).

The instant patient may undergo the determined prehabilitation plan 7010, which may include exercise, therapy, and other preparatory treatment supervised by a practitioner and/or performed without supervision. Throughout performance of the prehabilitation plan 7010, more preoperative data 1000 may be collected using the preoperative measurement system 100. For example, subsequent diagnostic imaging may be performed by diagnostic imaging systems 106, observations (e.g., by a supervisor, the patient, or a practitioner) about kinematics 1110 (e.g., movement), biometrics, lifestyle 1020, and/or psychosocial 1060 may be entered using user interfaces 104, and kinematics 1110 (e.g., movement), biometrics 1100, lifestyle 1020, and psychosocial 1060 data may be tracked using mobile devices 108 (e.g., bending motions, falls, activity level, heart rate, etc.).

During exercise performed as part of the prehabilitation plan 7010, the patient may wear wearable sensors 114 such as heart rate monitors and/or motion tracking systems (e.g., configured to be adhered to the skin) to further collect kinematics 1110 and biometrics 1100. In addition, motion sensor and/or kinesthetic sensing systems 114, which may include motion capture (mocap) systems and external motion sensors (e.g., using radar or light technology), may collect preoperative data 1000 during performance of the prehabilitation plan 7010.

As more preoperative data 1000 is collected throughout the preoperative period (e.g., during performance of the prehabilitation plan 7010), the determinations by the prehabilitation exercise algorithm 4010 and/or other preoperative algorithms 4000 and related intraoperative algorithms 5000. Throughout the preoperative period, the preoperative algorithms 4000 and/or related intraoperative algorithms 5000 may be continuously calculating, may be operated whenever new preoperative data 1000 is received, may periodically operate at predetermined time intervals, or may operate at the prompting of a practitioner or other input signal. As an alternative to separate preoperative algorithms and intraoperative algorithms 4000 and 5000, one composite or aggregate algorithm may be used and/or trained and/or one or more of these algorithms 4000, 5000 may be combined.

Similarly, during the intraoperative period, the intraoperative algorithms 5000 may operate simultaneously (or alternatively, at different times throughout the intraoperative period) and exchange inputs and outputs. For example, the BMD & Kinematics algorithm 5020 and the Multi-Joint Kinematics Assessment algorithm 5030 may use determinations from each other, also determinations (e.g., fall risk score) from the Fall Risk Detection algorithm 5020, and also determinations from the preoperative algorithms 4000 (e.g., stored in the memory system 40). The post-operative exercise program algorithm 5040 may further use determinations from the BMD & Kinematics algorithm 5020, Multi-Joint Kinematics Assessment algorithm 5030, and Fall Risk Detection algorithm 5020 as input to make determinations and implement processes. As an alternative to separate BMD & Kinematics algorithm 5020, Multi-Joint Kinematics Assessment algorithm 5030, Fall Risk Detection algorithm 5020, and post-operative algorithm 5040, one or more of these algorithms 5000 may be combined. As in the preoperative period, the intraoperative algorithms 5000 may be continuously calculating, may be operated whenever new intraoperative data 2000 is received, may periodically operate at predetermined time intervals, or may operate at the prompting of a practitioner or other input signal.

Similarly, during the postoperative period, the postoperative algorithms 6000 may operate simultaneously (or alternatively, at different times throughout the postoperative period) and exchange inputs and outputs. For example, the Postop Exercise Optimization Algorithm 6010, the Pain Medication Optimization Algorithm 6020, and the Patient Discharge Algorithm 6030 may use determinations from each other and also determinations from the preoperative algorithms 4000 and/or intraoperative algorithms 5000 (e.g., stored in the memory system 40). As an alternative to separate Postop Exercise Optimization Algorithm 6010, the Pain Medication Optimization Algorithm 6020, and the Patient Discharge Algorithm 6030, one or more of these algorithms 6000 may be combined. As in the preoperative and intraoperative periods, the postoperative algorithms 6000 may be continuously calculating, may be operated whenever new postoperative data 3000 is received, may periodically operate at predetermined time intervals, or may operate at the prompting of a practitioner or other input signal.

Aspects disclosed herein may be configured to optimize a “fit” or “tightness” of an implant provided to a patient during a surgical procedure. A fit of the implant may be made tighter by aligning the implant with a shallower bone slope and/or determining a shallower resulting or desired bone slope, by increasing a thickness or other dimensions of the implant, by determining certain types of materials or a type of implants or prosthesis (e.g., a stabilizing implant, a VVC implant, an ADM implant, or an MDM implant). A thickness of the implant may be achieved by increasing (or decrease) a size or shape of the implant. For example, with respect to FIGS. 11-13, a thickness of an implant 216 may be increased by increasing thickness, diameter, or other dimensions of at least portions the implant such as length of tibial stem 233 or femoral stem 236, thickness of tibial tray 235, humeral tray 246, or femoral implant 228, thickness of measurement device 244 or insert 230, diameter of ball joint 238 or glenoid sphere 248, etc.

Tightness may be impacted by gaps, which may be regulated by an insert which may vary depending on a type of implant or due to a motion. For example, to increase tightness by about one degree, a gap may be reduced by about 1 mm. In the case of a knee joint, tightness and/or gaps may be regulated by a tibial polyethylene insert which may vary from 9 millimeters (mm) to 25 mm depending on a type of implant or due to rotation of the components of the insert and/or implant. Gaps may be impacted by femoral and tibial cuts. Tightness may further be impacted by slope. A range of slope may be based on implant choice (for example, 0 degrees for a posterior stabilized or PS implant, 3 degrees for a cruciate retaining or CR implant), as well as surgical approach and patient anatomy.

A thickness of the implant may also be achieved by adding or removing a shim. For example, shims may be stackable and removable, and a thickness may be increased by adding one or more shims or adding a shim having a predetermined (e.g., above a certain threshold) thickness. For example, with reference to FIGS. 11-13, shims may be added to trays 235, 246, measurement device 244, or insert 230). The addition or subtraction of shims may be designed or configured to increase thickness and/or an alignment of the implant 216 or resulting steepness of a bone slope (e.g., tibial slope). Fit or rightness may also be achieved with certain types of bone cuts, bone preparations, or tissue cuts that reduce a number of cuts made and/or an invasiveness during surgery.

Aspects disclosed herein may be implemented during a robotic medical procedure where a robotic device, such as a surgical robot, a robotic tool manipulated or held by the surgeon and/or surgical robot, or other devices configured for automation perform at least a portion of a surgical procedure, such as a joint replacement procedure involving installation of an implant. Robotic device refers to surgical robot systems and/or robotic tool systems, and is not limited to a mobile or movable surgical robot. For example, robotic device may refer to a handheld robotic cutting tool, jig, burr, etc.

Aspects disclosed herein are not limited to specific scores, thresholds, etc. that are described. For example, outputs and/or scores disclosed herein may include other types of scores such as Hoos Koos, SF-12, SF-36, Harris Hip Score, etc.

Aspects disclosed herein are not limited to specific types of surgeries and may be applied in the context of osteotomy procedures, computer navigated surgery, neurological surgery, spine surgery, otolaryngology surgery, orthopedic surgery, general surgery, urologic surgery, ophthalmologic surgery, obstetric and gynecologic surgery, plastic surgery, valve replacement surgery, endoscopic surgery, and/or laparoscopic surgery.

Aspects disclosed herein may improve or optimize surgery outcomes. Aspects disclosed herein may augment the continuum of care to optimize post-operative outcomes for a patient. Aspects disclosed herein may recognize or determine previously unknown relationships, such as how injuries or movement at one joint affects movement at a different joint, to help optimize care, such as placement, type, and/or design of a prosthetic.

What is claimed is:

1. A method for optimizing a medical treatment plan, the method comprising:
receiving kinematics data from a wearable sensor coupled to an instant patient;
determining, based on the received kinematics data and stored information, a medical treatment plan for the instant patient; and
displaying the medical treatment plan on an electronic display, wherein:
the procedure includes installation of an implant,
determining the medical treatment plan includes determining an alignment, position, design, or type of the implant, and
the stored information includes:
(i) preoperative information for the instant patient, and
(ii) preoperative information, intraoperative information, and
postoperative information from a plurality of previous patients having at least one characteristic in common with the instant patient, each of the preoperative information, intraoperative information, and postoperative information including kinematics data obtained using a previous wearable sensor.

2. The method of claim 1, wherein the kinematics data includes:
a range of motion, stiffness, or laxity of a first joint; and
a range of motion, stiffness, or laxity of a second joint, wherein the implant is installed at the first joint.

3. The method of claim 2, further comprising determining that (i) the stiffness of the second joint is greater than a predetermined stiffness for the second joint or (ii) the laxity of the second joint is less than a predetermined laxity for the second joint, wherein determining the medical treatment plan includes determining whether the implant has a fit which is tighter than a predetermined fit based on (i) the determined stiffness of the second joint and/or (ii) the determined laxity of the second joint.

4. The method of claim 3, wherein determining the medical treatment plan includes determining whether:
a slope of a bone to which the implant is to be aligned is to be less than a predetermined slope,
a thickness of the implant is to be greater than a predetermined thickness,
a number of tissue and/or bone cuts used during the procedure is to be less than a predetermined number, and/or
the implant is to be a constrained type implant.

5. The method of claim 4, wherein the first joint is a knee joint, the second joint is a pelvic joint or hip joint, and determining the surgical plan includes:
determining that the implant is to be a valgus-valgus constrained (VVC) implant, and
determining whether the implant is to be aligned with a tibial slope less than or equal to a predetermined tibial slope.

6. The method of claim 1, wherein the kinematics data includes a range of motion, stiffness, or laxity of a first joint; and the method further includes receiving a bone density of a bone adjacent to the first joint.

7. The method of claim 4, further comprising:
determining whether the stiffness of the joint is below a predetermined stiffness threshold and/or that the laxity of the joint is above a predetermined laxity threshold; and
determining whether the bone density is less than a predetermined bone density threshold, wherein determining the medical treatment plan includes determining whether the implant has a fit which is tighter than a predetermined fit.

8. The method of claim 1, wherein the kinematics data includes:
postural sway or stability data of the instant patient,
a number or frequency of bending motions, squat motions, lunge motions, sit-to-stand motions, or one-legged motions performed by the instant patient over a period greater than one day, and/or
a number of fall events of the instant patient.

9. The method of claim 1, further comprising determining a fall risk score based on the kinematics data, wherein determining the medical treatment plan is based on the fall risk score.

10. The method of claim 9, further comprising determining whether the fall risk score is greater than a predetermined fall risk threshold, wherein determining the medical treatment plan includes determining that:
the implant is to be a constrained type of implant, and/or
a number of tissue and/or bone cuts during the procedure is to be less than a predetermined number.

11. The method of claim 1, further comprising determining, based on the received kinematics data and stored information, a prehabilitation plan for the instant patient.

12. The method of claim 11, wherein receiving the kinematics data includes receiving additional kinematics data from a performance of the prehabilitation plan.

13. The method of claim 12, wherein determining the prehabilitation plan includes determining, based on the additional kinematics data from the performance of the prehabilitation plan, a secondary prehabilitation plan.

14. The method of claim 12, wherein determining the medical treatment plan includes determining, based on the additional kinematics data, a secondary medical treatment plan.

15. The method of claim 1, further comprising:
determining, based on the received kinematics data, a patient readiness score; and
determining, based on the patient readiness score, (i) a timing of performing the medical treatment plan, (ii) a timing of performing a prehabilitation plan, and/or (iii) a timing of performing a rehabilitation plan.

16. The method of claim 1, further comprising determining, based on the received kinematics data and stored information, a rehabilitation plan for the instant patient.

17. The method of claim 1, further comprising receiving additional data during a performance of the medical treatment plan, wherein:
the implant includes one or more sensors, and
at least some of the additional data is received from the implant.

18. A method for optimizing a medical treatment plan, the method comprising:
receiving, after at least a portion of a medical treatment plan is performed, kinematics data from a sensored implant installed on an instant patient; and
determining, based on the received kinematics data and stored information, a secondary medical treatment plan for the instant patient, wherein:
determining the secondary medical treatment plan includes determining an adjusted alignment, position, design, or type of the sensored implant, and
the stored information includes:
(i) preoperative information for the instant patient, and
(ii) preoperative information, intraoperative information, and
postoperative information from a plurality of previous patients, wherein each of the preoperative information, intraoperative information, and postoperative information includes kinematics data obtained using a previous sensored implant.

19. The method of claim 18, wherein the medical treatment plan is for a total joint replacement surgery, and the sensored implant includes an inertial measurement unit (IMU).

20. A method for optimizing a medical treatment plan, the method comprising:
receiving primary data from a sensored implant installed on an instant patient during performance of the medical treatment plan;
receiving from a robotic device, after at least a portion of the medical treatment plan is performed, secondary data including at least one of biometrics data, incision length data, soft tissue integrity data, pressure data, and/or implant position data; and
determining, based on the primary data, the secondary data, and stored information, a secondary medical treatment plan for the instant patient, wherein:
determining the secondary medical treatment plan includes determining an alignment, position, design, or type of the sensored implant, and
the stored information includes:
(i) preoperative information for the instant patient, and
(ii) preoperative information, intraoperative information, and
postoperative information from a plurality of previous patients, wherein each of the preoperative information, the intraoperative information, and the postoperative information includes primary data obtained using a previous sensored implant and secondary data obtained using a previous robotic device.
